(19) United States
(12) Patent Application Publication
(10) Pub. No.: US 2026/0115275 A1
(43) Pub. Date: Apr. 30, 2026

(54) RIBOZYME-ACTIVATED RNA CONSTRUCTS AND USES THEREOF

(71) Applicant: The Regents of the University of California, Oakland, CA (US)

(72) Inventors: Prashant Mali, La Jolla, CA (US); Andrew Portell, La Jolla, CA (US); Amir Dailamy, La Jolla, CA (US); Aditya Kumar, La Jolla, CA (US)

(21) Appl. No.: 18/271,832

(22) PCT Filed: Jan. 11, 2022
(86) PCT No.: PCT/US2022/012004

§ 371 Date: Jul. 11, 2023

Related U.S. Application Data
(60) Provisional application No. 63/136,201, filed on Jan. 11, 2021.

(51) Int. Cl. A61K 39/215; A61K 31/7105; A61K 39/00; A61K 39/155; C12N 9/00; C12N 15/113

(52) U.S. Cl. CPC A61K 39/215; A61K 31/7105; A61K 39/155; C12N 9/93; C12N 15/113; C12Y 605/01003; A61K 2039/53; A61K 2039/60; C12N 2310/121; C12N 2750/14111; C12N 2800/10; C12N 2840/203

(57) ABSTRACT

The disclosure provides for ribozyme-mediated fusion constructs and systems and methods thereof, for use in a variety of applications, including for inducible gene expression systems, gene therapy, and combinatorial screening.

### CROSS REFERENCE TO RELATED APPLICATIONS

This application is a U.S. National Phase application filed under 35 U.S.C. § 371 and claims priority to International Application No. PCT/US2022/012004, filed Jan. 11, 2022, which application claims priority under 35 U.S.C. § 119 from Provisional Application Ser. No. 63/136,201, filed Jan. 11, 2021, the disclosures of which are incorporated herein by reference.

### STATEMENT OF GOVERNMENT SUPPORT

This invention was made with Government support under Grant Nos. R01GM123313, R01CA222826, and R01HG009285, awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Feb. 16, 2022, is named 00015-396WO1_SL.txt and is 633,808 bytes in size.

### TECHNICAL FIELD

The disclosure provides for ribozyme-mediated fusion constructs and systems and methods thereof, for use in a variety of applications, including for inducible gene expression systems, gene therapy, and combinatorial screening.

### BACKGROUND

Ribozymes (ribonucleic acid enzymes) are RNA molecules that have the ability to catalyze specific biochemical reactions, including CRNA splicing in gene expression, similar to the action of protein enzymes. Ribozymes participate in a variety of RNA processing reactions, including RNA splicing, viral replication, and transfer RNA biosynthesis. Examples of ribozymes include the hammerhead ribozyme, the VS ribozyme, Leadzyme and the hairpin ribozyme. Ribozymes have been proposed and developed for the treatment of disease through gene therapy.

### SUMMARY

The disclosure provides for innovative engineering of endogenous RNA processing machinery to create linked RNA fusion constructs which can be utilized for RNA-based readouts for combinatorial genetic interaction screens as well as inducible gene expression. As shown in the examples herein, separately transcribed sequences with complementarity to one another are fused to self-cleaving ribozymes. Once transcribed, the auto-catalytic activity of the ribozymes cleaves the transcripts to create unique ends. Due to the complementary region, the two transcripts will then hybridize, juxtaposing the cleaved ends which can then be recognized by endogenous RNA ligases to create a linked fusion construct. It is further demonstrated herein the applicability of this approach to link two transcripts delivered to cells on disparate library elements. When linked to intronic sequences, the ribozyme-mediated RNA-fusion constructs can be utilized for controllable expression of full-length gene products. The fusion of RNAs (including for circularization) had additional utility in transcriptome and genome engineering modalities, such as RNAi, ASOs, ADAR-recruiting guide RNAs, and guide RNAs in CRISPR-Cas.

The disclosure provides a ribozyme activated RNA-construct(s) comprising one or more ribozymes; and one or more RNA coding sequences for at least one polypeptide of interest, wherein the transcription of the one or more RNA coding sequences for at least one polypeptide of interest is activated by or dependent upon the activity of the one or more ribozymes. In one embodiment, the ribozyme activated RNA-construct further comprises a first engineered RNA element comprising an optional primer region, an optional barcode region, an RNA coding sequence for a polypeptide of interest and a complementary sequence to a sequence of a second engineered RNA element, and a first self-cleaving ribozyme; a second engineered RNA element comprising an optional primer region, an optional barcode region, an RNA coding sequence for a polypeptide of interest and a complementary sequence to a sequence of the first engineered RNA element, and a second self-cleaving ribozyme; wherein cleavage of the first and second engineered RNA elements by the first and second self-cleaving ribozymes, respectively, provides for a hybridization construct that comprises a region of dsRNA from the commentary sequences being hybridized together, wherein the hybridization construct can be further ligated by an RNA ligase to form an RNA-fusion construct, and wherein expression from the RNA-fusion construct produces the at least one polypeptide of interest. In a further embodiment, the first engineered RNA element comprises a barcode sequence or a unique molecular identity (UMI) sequence and/or wherein the second engineered RNA element comprises a barcode sequence or a UMI sequence. In still a further embodiment, the barcode sequence or the UMI sequence of the first engineered element has a different sequence than the barcode region or the UMI sequence from the second engineered RNA element. In another embodiment, the first engineered RNA element comprises a primer sequence, and/or wherein the second engineered RNA element comprises a primer sequence. In a further embodiment, the primer sequence of the first engineered RNA element is different from the primer sequence from the second engineered RNA element. In another embodiment, the first and second complementary sequences are from 30 to 60 bp in length. In a further embodiment, the first and second complementary sequence are from 40 to 50 bp in length. In another embodiment, the first and second ribozymes are Twister ribozymes. In a further embodiment, the first ribozyme is a P3 Twister ribozyme. In still a further embodiment, the second ribozyme is a P1 Twister ribozyme. In another embodiment, the RNA ligase is RtcB. In another or further embodiment of any of the foregoing embodiments, a vector or plasmid comprises the first engineered element, wherein the first engineered element is located downstream of a first RNA promoter and a first perturbation element; and/or wherein a vector or plasmid comprises the second engineered element, wherein the second engineered element is located downstream of a second RNA promoter and a second perturbation element. In a further embodiment, the first RNA promoter and/or the second RNA promoter is a polymerase III promoter. In yet a further embodiment, the polymerase III promoter is a hU6 promoter. In another embodiment, the first perturbation element and/or the second perturbation element is a sgRNA utilized in a CRISPR knockout screen. In another embodiment, a first engineered RNA element comprising an RNA coding sequence for a polypeptide of interest, an intron sequence, a complementary sequence to a sequence of a second engineered RNA element and a 3′ aptamer, and a first self-cleaving ribozyme, and wherein the 3′ aptamer interacts with a first self-cleaving ribozyme to stabilize it; a second engineered RNA element comprising an RNA coding sequence for a polypeptide of interest, an intron sequence, a complementary sequence to a sequence of the first engineered RNA element, and a 3′ aptamer, wherein the second engineered RNA template is tethered to a second self-cleaving ribozyme, and wherein the 3′ aptamer interacts with a second self-cleaving ribozyme to stabilize it; wherein cleavage of the first and second engineered RNA elements by the first and second self-cleaving ribozymes, respectively, provides for a hybridization construct that comprises a region of dsRNA from the commentary sequences being hybridized together, and wherein the hybridization construct can be further ligated by an RNA ligase and the intron sequences removed by a spliceosome to form an RNA-fusion construct, wherein expression from the RNA-fusion construct produces the at least one polypeptide of interest. In a further embodiment, the intron sequence is derived from dihydrofolate reductase. In another embodiment, the RNA coding sequences for a polypeptide of interest are adjacent to each of the intron sequences. In another embodiment, the RNA coding sequences for a polypeptide of interest encode a polypeptide/protein selected from insulin, clotting factor IX, the cystic fibrosis transmembrane conductance regulator protein, and the dystrophin protein. In another embodiment, the ribozyme activated RNA-construct(s) comprises one or more promoter sequences; one or more RNA coding sequences for at least one polypeptide of interest; one or more ribozymes, wherein the one or more ribozymes are aptazyme-based riboswitches; a 3′ UTR sequence comprising the aptazyme-based riboswitches; and a poly(A) sequence; wherein the aptazyme-based riboswitches when not bound to target ligands destabilize the ribozyme activated RNA-construct(s) leading to decreased expression of the at least polypeptide of interest, and wherein the aptazyme-based riboswitches when bound to target ligands stabilize the ribozyme activated RNA-construct(s) leading to increased expression of the at least polypeptide of interest. In another embodiment, the aptazyme-based riboswitches are hammerhead aptazymes. In still another embodiment, the target ligands are selected from tetracycline, theophylline, and guanine. In another embodiment, the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide. In a further embodiment, the biological response modifier or an immunopotentiating cytokine. In yet a further embodiment, the immunopotentiating cytokine is selected from the group consisting of interleukins 1 through 38, interferon, tumor necrosis factor (TNF), and granulocyte-macrophage-colony stimulating factor (GM-CSF). In another embodiment, the 2A- or 2A-like peptide further comprises a GSG linker moiety. In yet another embodiment, the genome editing enzyme is selected from the group consisting of a zinc finger nuclease, a transcription activator-like effector nuclease (TALEN), an engineered meganuclease and an RNA-guided DNA endonuclease (Cas) polypeptide. In still another embodiment, the one or more promoter sequences are polymerase II (pol-II) promoter sequences. In another embodiment, the one or more promoter sequences have a sequence(s) for EF1α, hU6, SV40, CMV, a RSV, NEUROD2 and/or TBX20. In another embodiment, the poly(A) sequence is a bGH poly(A) sequence.

The disclosure also provides a pharmaceutical composition comprising the ribozyme activated RNA-construct(s) as described above, wherein the ribozyme activated RNA-construct(s) is linearized and comprises a 5′ ribozyme; a 5′ ligation sequence; an internal ribosome entry site (IRES) sequence; an RNA coding sequence for at least one polypeptide of interest; a 3′ ligation sequence; and a 3′ ribozyme sequence, and a pharmaceutically acceptable carrier. In a further embodiment, the linear ribozyme activated RNA-construct(s) lacks a polymerase binding region. In still another embodiment, the 5′ and 3′ ribozymes are selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing. In another embodiment, the 5′ and 3′ ligation sequences are substrates of naturally occurring ligases in situ. In a further embodiment, the naturally occurring ligase is RtcB. In another embodiment, the IRES comprises a sequence of any one of the sequences of SEQ ID NO: 1-1328 and sequences thereof wherein T is U. In another embodiment, the at least one polypeptide of interest comprises two or more polypeptides of interest separated by a self-cleaving peptide. In still a further embodiment, the self-cleaving peptide comprises a 2A- or 2A-like-peptide. In another embodiment, the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide. In yet another embodiment, the biological response modifier or an immunopotentiating cytokine. In a further embodiment, the immunopotentiating cytokine is selected from the group consisting of interleukins 1 through 38, interferon, tumor necrosis factor (TNF), and granulocyte-macrophage-colony stimulating factor (GM-CSF). In another embodiment, the 2A- or 2A-like peptide further comprises a GSG linker moiety. In yet another embodiment, the genome editing enzyme is selected from the group consisting of a zinc finger nuclease, a transcription activator-like effector nuclease (TALEN), an engineered meganuclease and an RNA-guided DNA endonuclease (Cas) polypeptide. In still another embodiment, the 5′ and 3′ ribozyme sequences are independently selected from a sequence that is at least 85-100% identical to 5′-GCCATCAGTCGCCGGTCCCAAGCCCGGATAAAATGGGAGGGGGCGGGAAACCGCCT-3′ (SEQ ID NO: 1354) or 5′-AACACTGCCAATGCCGGTCCCAAGCCCGGATAAAAGTGGAGGGTACAGTCCACGC-3′ (SEQ ID NO: 1355) and either of the foregoing sequences wherein T is U. In another embodiment, the 5′ and 3′ ligation sequences are independently selected from a sequence that is at least 85-100% identical to 5′-AACCATGCCGACTGATGGCAG-3′ (SEQ ID NO: 1356) or 5′-CTGCCATCAGTCGGCGTGGACTGTAG-3′ (SEQ ID NO: 1357) and either of the foregoing wherein T is U. In another embodiment, the IRES sequence is at least 85-100% identical to 5′-gcggccgcgtcgacgggcccgcggaattccgccccccccccctctccctcccccccccctaacgttac tggccgaagccgcttggaataaggccggtgtgcgtttgtctatatgttattttccaccatattgccgt cttttggcaatgtgagggcccggaaacctggccctgtcttcttgacgagcattcctaggggtctttcc cctctcgccaaaggaatgcaaggtctgttgaatgtcgtgaaggaagcagttcctctggaagcttcttg aagacaaacaacgtctgtagcgaccctttgcaggcagcggaaccccccacctggcgacaggtgcctct gcggccaaaagccacgtgtataagatacacctgcaaaggcggcacaaccccagtgccacgttgtgagt tggatagttgtggaaagagtcaaatggctctcctcaagcgtattcaacaaggggctgaaggatgccca gaaggtaccccattgtatgggatctgatctggggcctcggtgcacatgctttacatgtgtttagtcga ggttaaaaaaacgtctaggccccccgaaccacggggacgtggttttcctttgaaaaacacgatgataa tatggccacaacc-3′ (SEQ ID NO: 1358) and a sequence of the foregoing wherein T is U.

The disclosure also provides a vaccine composition comprising the ribozyme activated RNA-construct(s) of the disclosure, wherein the ribozyme activated RNA-construct(s) is linearized and comprises a 5′ ribozyme; a 5′ ligation sequence; an internal ribosome entry site (IRES) sequence; an RNA coding sequence for at least one antigenic polypeptide; a 3′ ligation sequence; and a 3′ ribozyme sequence, and a pharmaceutically acceptable carrier. In a further embodiment, the linearized ribozyme activated RNA-construct(s) lacks a polymerase binding region. In another embodiment, the 5′ and 3′ ribozyme is selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing. In another embodiment, the 5′ and 3′ ligation sequences are substrates of naturally occurring ligases in situ. In a further embodiment, the naturally occurring ligase is RtcB. In another embodiment, the IRES comprises any one of the sequences of SEQ ID NO: 1-1328 and sequences thereof wherein T is U. In another embodiment, the at least one antigenic polypeptide comprises two or more antigenic polypeptides separated by a self-cleaving peptide. In a further embodiment, the self-cleaving peptide comprises a 2A- or 2A-like-peptide. In a further embodiment, the 2A- or 2A-like peptide further comprises a GSG linker moiety. In still another embodiment, the 5′ and 3′ ribozyme sequences are independently selected from a sequence that is at least 85-100% identical to 5′-GCCATCAGTCGCCGGTCCCAAGCCCGGATAAAATGGGAGGGGGCGGGAAACCGCCT-3′ (SEQ ID NO: 1354) or 5′-AACACTGCCAATGCCGGTCCCAAGCCCGGATAAAAGTGGAGGGTACAGTCCACGC-3′ (SEQ ID NO: 1355) and sequences of either of the foregoing wherein T is U. In another embodiment, the 5′ and 3′ ligation sequences are independently selected from a sequence that is at least 85-100% identical to 5′-AACCATGCCGACTGATGGCAG-3′ (SEQ ID NO: 1356) or 5′-CTGCCATCAGTCGGCGTGGACTGTAG-3′ (SEQ ID NO: 1357) and sequences of either of the foregoing wherein T is U. In another embodiment, the IRES sequence is at least 85-100% identical to 5′-gcggccgcgtcgacgggcccgcggaattccgccccccccccctctccctcccccccccctaacgttac tggccgaagccgcttggaataaggccggtgtgcgtttgtctatatgttattttccaccatattgccgt cttttggcaatgtgagggcccggaaacctggccctgtcttcttgacgagcattcctaggggtctttcc cctctcgccaaaggaatgcaaggtctgttgaatgtcgtgaaggaagcagttcctctggaagcttcttg aagacaaacaacgtctgtagcgaccctttgcaggcagcggaaccccccacctggcgacaggtgcctct gcggccaaaagccacgtgtataagatacacctgcaaaggcggcacaaccccagtgccacgttgtgagt tggatagttgtggaaagagtcaaatggctctcctcaagcgtattcaacaaggggctgaaggatgccca gaaggtaccccattgtatgggatctgatctggggcctcggtgcacatgctttacatgtgtttagtcga ggttaaaaaaacgtctaggccccccgaaccacggggacgtggttttcctttgaaaaacacgatgataa tatggccacaacc-3′ (SEQ ID NO: 1358) and sequences of the foregoing wherein T is U. In another embodiment, the antigenic polypeptide comprises a SARS-COV-2 spike protein. In another embodiment, the at least one polypeptide of interest or antigenic polypeptide is contained within a self-amplifying RNA construct. In a further embodiment, the self-amplifying RNA construct comprises an alphavirus or a Paramyxovirus.

The disclosure also provides a plasmid or capsid comprising the ribozyme activated RNA-construct(s) of the disclosure. In one embodiment, the plasmid or capsid is an AAV-based plasmid or capsid. In another embodiment, the plasmid expresses a Cas9 protein and a gRNA.

The disclosure also provides a combinatorial screen comprising the ribozyme activated RNA-construct(s) of any of the foregoing embodiments.

In a particular embodiment, the disclosure provides a ribozyme-mediated RNA-fusion construct or system comprising: a first engineered RNA element comprising a primer region, a barcode region, and a complementary sequence to a sequence of a second engineered RNA element, wherein the first engineered RNA template is tethered to a first self-cleaving ribozyme; a second engineered RNA element comprising a primer region, a barcode region, and a complementary sequence to a sequence of the first engineered RNA element, wherein the second engineered RNA template is tethered to a second self-cleaving ribozyme; wherein cleavage of the first and second engineered RNA elements by the first and second self-cleaving ribozymes, respectively, provides for a hybridization construct that comprises a region of dsRNA from the commentary sequences being hybridized together, and wherein the hybridization construct can be further ligated by an RNA ligase to form an RNA-fusion construct. In a further embodiment, the first engineered RNA element comprises a barcode region that has a different sequence than the barcode region from the second engineered RNA element. In yet a further embodiment, the first engineered RNA element comprises a primer region that has a different sequence than the primer region from the second engineered RNA element. In another embodiment, the first and second complementary sequence are from 30 to 60 bp in length. In yet another embodiment, the first and second complementary sequence are from 40 to 50 bp in length. In a further embodiment, the first and second ribozymes are Twister ribozymes. In yet a further embodiment, the first ribozyme is a P3 Twister ribozyme. In another embodiment, the second ribozyme is a P1 Twister ribozyme. In a further embodiment, the RNA ligase is RtcB.

In another embodiment, the disclosure also provides a vector or plasmid comprising a first engineered element of described herein, wherein the first engineered element is located downstream of a first RNA promoter and a first perturbation element. In yet another embodiment, the disclosure also provides a vector or plasmid, comprising the second engineered element described herein, wherein the second engineered element is located downstream of a second RNA promoter and a second perturbation element. In a further embodiment, the first RNA promoter and/or the second RNA promoter is a polymerase III promoter. In yet a further embodiment, the polymerase III promoter is hU6 promoters. In a certain embodiment, the first perturbation element and/or the second perturbation element is a sgRNA utilized in a CRISPR knockout screen.

In a particular embodiment, the disclosure further provides for a combinatorial screen comprising the vector or plasmid described herein.

In a certain embodiment, the disclosure provides an inducible ribozyme-mediated RNA-fusion construct or system comprising: a first engineered RNA element comprising an intron sequence, a complementary sequence to a sequence of a second engineered RNA element and a 3′ aptamer, wherein the first engineered RNA template is tethered to a first self-cleaving ribozyme, and wherein the 3′ aptamer interacts with a first self-cleaving ribozyme to stabilize it; a second engineered RNA element comprising an intron sequence, a complementary sequence to a sequence of the first engineered RNA element, and a 3′ aptamer, wherein the second engineered RNA template is tethered to a second self-cleaving ribozyme, and wherein the 3′ aptamer interacts with a second self-cleaving ribozyme to stabilize it; wherein cleavage of the first and second engineered RNA elements by the first and second self-cleaving ribozymes, respectively, provides for a hybridization construct that comprises a region of dsRNA from the commentary sequences being hybridized together, and wherein the hybridization construct can be further ligated by an RNA ligase to form an RNA-fusion construct. In a further embodiment, the intron sequence is derived from dihydrofolate reductase. In yet a further embodiment, portions of therapeutic genes or proteins are fused to each of the intron sequences. In another embodiment, the therapeutic genes or proteins are selected from the human insulin gene, clotting factor IX, the cystic fibrosis transmembrane conductance regulator protein, and the dystrophin protein.

### DESCRIPTION OF DRAWINGS

FIG. 1A-C provides the results from preliminary studies. (A) Schematic illustrating the fusion of disparate barcodes at the RNA level. (B) Gel electrophoresis image following RT-PCR of plasmid transfection of fragL and fragR either alone or in combination. (C) Sanger sequencing trace of purified PCR product (SEQ ID NO: 1359) shown in panel B compared to the expected fusion (SEQ ID NO: 1347).

FIG. 2A-E provides (A) schematic illustrating the plasmid design for a combinatorial screen in which the ribozyme, complementary linker, barcode and primer of the ribozyme-mediated RNA-fusion construct are cloned downstream of a perturbation such as an sgRNA utilized in a CRISPR knockout screen. (B) Gel electrophoresis of an RT-PCR following plasmid transfection of HEK293T cells with the sgRNA fragL and sgRNA fragR constructs alone or in combination. (D) Gel electrophoresis of an RT-PCR following plasmid transfection of HEK 293T cells with the antisense fragL and antisense fragR constructs alone or in combination (SEQ ID NO: 1348). (C and E) Sanger sequencing trace of the purified PCXR product from the antisense-oriented RNA fusion (SEQ ID NO: 1349). FIG. 2 discloses SEQ ID NOS 1348, 1348-1349, and 1360, respectively, in order of appearance.

FIG. 3 provides a schematic illustrating the ribozyme-mediated RNA fusion approach for inducible gene expression. A therapeutic payload is split between two constructs. The fragL construct contains the N-terminus of the protein fused to an intronic sequence, a complementary region, a self-cleaving ribozyme, a complementary region, an intronic sequence, and the C-terminus of the therapeutic protein. Upon addition of an aptamer-binding ligand, the tertiary interactions between the aptamer and ribozyme are disrupted, allowing for autocatalytic cleavage of the transcript. The complementary regions hybridize to one another and the generated ends are ligated together by the endogenous RNA-ligase, RtcB. With the intronic sequences juxtaposed to one another, the cellular splicing machinery can recognize the splice sites and create a full-length functional protein.

FIG. 4 presents a schematic of RNA fusion from PolII promoters. Two constructs are cloned into the px600 AAV backbone which contains two halves of green fluorescent protein (GFP). Each GFP is linked to a DHFR intron and contains the RNA fusion linker (45 bp complementary region) as well as either the P3 (GFP-L) or P1 (GFP-R) Twister Ribozyme. Upon transcription, the ribozymes undergo self-cleavage to generate 5′ hydroxyl and 2′,3′-cyclic phosphate ends. The complementary linker regions will then hybridize, be ligated by RtcB, bringing the intronic sequences near one another. The endogenous spliceosome machinery will then spice out the introns to generate a fluorescent GFP molecule.

FIG. 5A-C presents (A) fluorescent images taken 48 hours after transfection of HEK293FT cells with the plasmids containing the RNA fusion machinery linked to one half of GFP (GFP-Left or GFP-Right) and an intronic sequences that are recognized by the spliceosome. (B) RNA was isolated from the HEK293FT cells shown in panel A and RT-PCR was performed using primers on both halves of the GFP transcript. (C) The PCR product was purified and Sanger sequenced (SEQ ID NO: 1350) to confirm the proper ligation of the full-length protein. FIG. 5C discloses SEQ ID NOS 1350 and 1350, respectively, in order of appearance.

FIG. 6A-E presents (A) fluorescent images taken 48 hours after transfection of HEK293FT cells with the plasmids containing the RNA fusion machinery linked to one half of GFP (GFP-Left or GFP-Right) and different intronic sequences. (B) Flow cytometry was run on transfected cells after 48 hours and the percent of GFP+ cells was quantified. (C) RT-qPCR relative expression of GFP with the DHFR intron after 48 hours. (D) RT-qPCR relative expression of GFP with the pCI constructs after 48 hours. (E) The activation ratio was calculated from qPCR comparing the relative GFP expression levels when the complementary sequence was present.

FIG. 7 provides the results of cells isolated 48 hours after transfection and analyzed via flow cytometry. Shown is the percentage of GFP+ cells from the total cells present.

FIG. 8A-B shows (A) schematic of a plasmid design for the ribozyme-mediated RNA barcode fusion constructs driven by the polymerase-II and polymerase-III-like H1 promoter; (B) Relative expression, as determined by quantitative RT-PCR (qRT-PCR) of the RNA fusion construct (normalized to GAPDH) for cells transfected with either a negative control (lentiCRISPRv2 plasmid backbone) or both the fragL and fragR constructs driven by a U6- or H1-promoter.

FIG. 9A-B provides (A) schematic illustrating the design for ribozyme-mediated circularized RNA barcodes. The 3′ end of the fragL construct is modified with a ‘designer exon’ and an intron, while the 5′ end of the fragR construct is mediated with an intron followed by a “designer exon’. Once transcribed, the ribozymes will self-cleave, the complementary sequences will hybridize, which juxtaposes the designer exons and introns with one another. This will then be recognized by the splicesome and a back-spicing reaction will take place, splicing out the introns and joining the exons for a fully circularized construct; (B) agarose gel image from an RT-PCR assay utilizing the primer pairs illustrated in panel (A) on cells transfected with the circ-fragL and circ-fragR constructs.

FIG. 10 sequences of the designer exon with the exon splicing enhancer (ESE) elements labeled, sequences of the intron sequences used with the fragL and fragR constructs with important features highlighted (SEQ ID Nos: 1351-1353).

FIG. 11A-B shows (A) a schematic illustrating the plasmid design for the dual-promoter system in which the GFP-pCI-L construct is driven by the U6 promoter while the GFP-pCI-R construct is driven by the CMV promoter; and (B) Relative expression of the GFP RNA transcript as determined by qRT-PCT. The fold activation between the full length and the link free construct without the ribozyme and complementary sequence is show above the two graphs.

FIG. 12A-C shows (A) a schematic illustrating the mechanism of inducible gene expression using a tetracycline-responsive hammerhead aptazyme embedded into the 3′ UTR of the gene of interest (GOI); (B) Insulin ELISA absorbance values from cell culture supernatant collected 48 hours after transfection with DMEM (−) or tetracycline (+) added 4-6 hours after transfection; and (C) Insulin ELISA absorbance values form cell culture supernatant collected 48 hours after transfection. Increasing concentrations of tetracycline were added 4-6 hours after transfection.

FIG. 13A-B shows (A) a schematic of the various ribozyme positions which have been assessed for their effect on gene expression induction; and (B) cell culture supernatant insulin ELISA levels for the plasmid design shows in panel (A) 48 hours after transfection in HEK293T cells.

FIG. 14A-E shows (A) a schematic of the plasmid design for the ribozyme mediated inducible SaCas9 construct; (B) SaCas9 mRNA levels as measured by quantitative PCR 48 hours after transfection in HEK293T cells; (C) timeline of experiment performed to assess tetracycline-induced Cas9 mediated editing of the Pcsk9 gene. Arrows denote the timepoints in which blood was collected (B), tetracycline was administered (T) on 3 consecutive days, or where the livers were harvested (H); (D) PCSK9 serum levels measured at various timepoints following AAV8 administration; and (E) editing rate measured in the livers of mice injected with AAV8-SaCas9-Ribo constructs at the 5-week and 7-week timepoints.

FIG. 15 schematically shows an in vitro transcribed RNA delivery system wherein a linear RNA construct is circularized in situ. In this embodiment, in vitro linear RNA is generated, the linear RNA is delivered into cells, and in situ it circularizes. In some embodiments, only in situ does it circularize.

FIG. 16A-D provide (A) a general template for engineering circular RNA (SEQ ID NO: 1329) wherein the payload is provided as GFP but can be any polypeptide of interest; (B) a schematic of the circularized RNA construct and resulting GFP expression comparing linear and circular constructs on Day 1, 2 and 3; (C) micrographs of fluorescent expression and expression; and (D) relative GFP RNA expression over time.

FIG. 17A-C provides (A) schematic of a circular format containing exemplary payloads of CRISPR/ZF/TALEs/Genes; (B) a graph showing editing efficiency for linear vs. circular constructs containing Zinc Finger (ZF) protein; and (C) a graph showing editing efficiency for constructs containing Cas9 protein.

FIG. 18 shows a design of a construct to generate in situ circularized RNA containing a self-amplifying RNA construct (SEQ ID NO: 1330).

FIG. 19 provides sequence of IRESs (Table 2) useful in the methods and compositions of the disclosure.

FIG. 20 provides sequence of circular constructs useful in the methods and compositions of the disclosure (SEQ ID Nos: 1331-1342 and 1344-1346, respectively, in order of appearance).

### DETAILED DESCRIPTION

As used herein and in the appended claims, the singular forms “a,” “an,” and “the” include plural referents unless the context clearly dictates otherwise. Thus, for example, reference to “a prodrug” includes a plurality of such prodrugs and reference to “the chemotherapeutic agent” includes reference to one or more chemotherapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

As used herein, the term “alphavirus” has its conventional meaning in the art, and includes the various species such as Venezuelan Equine Encephalitis (VEE) Virus, Eastern Equine Encephalitis (EEE) virus, Everglades Virus (EVE), Mucambo Virus (MUC), Pixuna Virus (PIX), and Western Equine Encephalitis Virus, all of which are members of the VEE/EEE Group of alphaviruses. Other alphaviruses include, e.g., Semliki Forest Virus (SFV), Sindbis, Ross River Virus, Chikungunya Virus, S.A. AR86, Barmah Forest Virus, Middleburg Virus, O'nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus. Alphaviruses particularly useful in the constructs and methods described herein are VEE/EEE group alphaviruses.

The terms “alphavirus RNA replicon”, “alphavirus replicon RNA”, “alphavirus RNA vector replicon”, “vector replicon RNA” and “self-replicating RNA construct” are used interchangeably to refer to an RNA molecule expressing nonstructural protein genes such that it can direct its own replication (amplification) and comprises, at a minimum, 5′ and 3′ alphavirus replication recognition sequences, coding sequences for alphavirus nonstructural proteins, and a polyadenylation tract. It may additionally contain one or more elements (e.g., IRES sequences, core or mini-promoters, 2A peptide sequence and the like) to direct the expression, meaning transcription and translation, of a coding sequence of interest. The alphavirus replicon of the disclosure can comprise, in one embodiment, 5′ and 3′ alphavirus replication recognition sequences, coding sequences for alphavirus nonstructural proteins, a polyadenylation tract.

The term “adeno-associated virus” or “AAV” as used herein refers to a member of the class of viruses associated with this name and belonging to the genus depend parvovirus, family Parvoviridae. Multiple serotypes of this virus can be suitable for gene delivery. In some cases, serotypes can infect cells from various tissue types. Examples of AAV serotypes are AAV1, AAV2, AAV3, AAV4, AAV5, AAV6, AAV7, AAV8, AAV9, AAV10, and AAV11. Non-limiting exemplary serotypes useful for the purposes disclosed herein include any of the 11 serotypes, e.g., AAV2 and AAV8.

As used herein, the term “circularized” and/or “circular” used in the context of a nucleic acid molecule (e.g., an engineered guide RNA) can generally refer to a nucleic acid molecule that can be represented as a polynucleotide sequence in a circular 2-dimensional format with one nucleotide after the other wherein the represented polynucleotide is circular or a closed loop. In some embodiments, a circular nucleic acid molecule does not comprise a 5′ reducing hydroxyl, a 3′ reducing hydroxyl, or both capable of being exposed to a solvent

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 808, 90%, or 100% complementarity to a specified second nucleotide sequence, indicating that 8 of 10, 9 of 10 or 10 of 10 nucleotides of a sequence are complementary to the specified second nucleotide sequence.

The term “encode” as it is applied to polynucleotides can refer to a polynucleotide which is said to “encode” a polypeptide if, in its native state or when manipulated, it can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological or cellular material having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology can refer to a percent (%) identity of a sequence to a reference sequence. As a practical matter, whether any particular sequence can be at least 508, 608, 708, 808, 85%, 90%, 92%, 95%, 96%, 97%, 98% or 99% identical to any sequence described herein, such particular peptide, polypeptide or nucleic acid sequence can be determined conventionally using computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, a sequence of the disclosure) and a subject sequence, also referred to as a global sequence alignment, can be determined using the FASTDB computer program. In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 108 is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 08 to about 258; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 308 to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells, or tissues.

A “ligation sequence” refers to a sequence complementary to another sequence, which enables the formation of Watson-Crick base pairing to form suitable substrates for ligation by a ligase, e.g., an RNA ligase. In one embodiment, a 5′ ligation sequence and a 3′ ligation sequence are substrates for an RNA ligase such as, but not limited to RtcB. The 5′ and 3′ ligation sequences when ligated circularize an RNA molecule of the disclosure. Such circularization reduces RNA degradation and improves persistence in vivo.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence.

The terms “polynucleotide” and “oligonucleotide” are used interchangeably and refer to a polymeric form of nucleotides of any length, either deoxyribonucleotides or ribonucleotides or analogs or combinations thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form. In some embodiments, a polynucleotide can include both RNA and DNA nucleotides.

The term “polynucleotide sequence” can be the alphabetical representation of a polynucleotide molecule. This alphabetical representation can be input into databases in a computer having a central processing unit and used for bioinformatics applications such as functional genomics and homology searching. In any alphabetic representation, the disclosure contemplates both RNA and DNA (wherein “T” is replaced with “U” or vice-a-versa).

In certain embodiments, “promoters” may be used to drive transcription of an operably linked nucleic acid. As used herein “promoter” refers to a DNA sequence which contains the binding site for RNA polymerase and initiates transcription of a downstream nucleic acid sequence. A promoter for use in the disclosure can be a constitutive, inducible or tissue specific, or a temporal promoter. Suitable promoters can be derived from viruses, prokaryotes and eukaryotes. Suitable promoters can be used to drive expression by any RNA polymerase. Examples of inducible promoters include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, and the like. Inducible promoters can be regulated by various molecules such as doxycycline. In one embodiment, the promoter is a prokaryotic promoter selected from the group consisting of T7, T3, SP6 and derivatives thereof.

As used herein, a “ribozyme” (ribonucleic acid enzyme) is an RNA molecule capable of catalyzing biochemical reactions. A “self-cleaving ribozyme” is a ribozyme capable of cleaving itself. The ribozyme used in the disclosure can be any small endonucleolytic ribozyme that will self-cleave in the target cell type including, for example, hammerhead, hairpin, the hepatitis delta virus, the Varkud satellite, twister, twister sister, pistol and hatchet. See, e.g., Roth et al., Nat Chem Biol. 10 (1): 56-60; and Weinberg et al., Nat Chem Biol. 2015 August; 11 (8): 606-10, both incorporated herein by reference. U.S. 2015/0056174 provides modified hammerhead ribozymes with enhanced endonucleolytic activity. Ribozymes cleave the substrate RNA in a sequence specific manner at a substrate cleavage site. Typically, a ribozyme contains a catalytic region flanked by two binding regions. The ribozyme binding regions hybridize to the substrate RNA, while the catalytic region cleaves the substrate RNA at a substrate cleavage site to yield a cleaved RNA product. In various embodiment, the 5′ or 3′ of various constructs can be a Twister ribozyme or a Twister Sister ribozyme. For example, the 5′ and 3′ ribozymes of various constructs are either a P3 or P1 Twister ribozyme but not both P3 or both P1.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N. Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2nd ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N. Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder. Also, the terms “treat”, “treating” and “treatment” include preventing or delaying the onset of the disease or disorder symptoms, and/or lessening the severity or frequency of symptoms of the disease or disorder.

As used herein, the term “vector” can refer to a nucleic acid construct deigned for transfer between different hosts, including but not limited to a plasmid, a virus, a cosmid, a phage, a bacterial artificial chromosome (BAC), a yeast artificial chromosome (YAC), etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves. Alphavirus vectors, such as Semliki Forest virus-based vectors and Sindbis virus-based vectors, have also been developed for use in gene therapy and immunotherapy. In aspects where gene transfer is mediated by a retroviral vector, a vector construct can refer to the polynucleotide comprising the retroviral genome or part thereof, and a gene of interest.

The disclosure describes implementations of engineered endogenous RNA processing machinery to create linked RNA fusion constructs which can be utilized for RNA-based readouts for combinatorial genetic interaction screens as well as inducible gene expression. Separately transcribed sequences, with complementarity to one another, are fused to self-cleaving ribozymes. Once transcribed, the auto-catalytic activity of the ribozymes cleaves the transcripts to create unique ends. Due to the complementary region, the two transcripts will then hybridize, juxtaposing the cleaved ends which can then be recognized by endogenous RNA ligases to create a linked fusion construct. The disclosure further demonstrates the applicability of this approach to link two transcripts delivered to cells on disparate library elements, and when linked to intronic sequences, the RNA-fusion constructs can be utilized for controllable expression of full-length gene products.

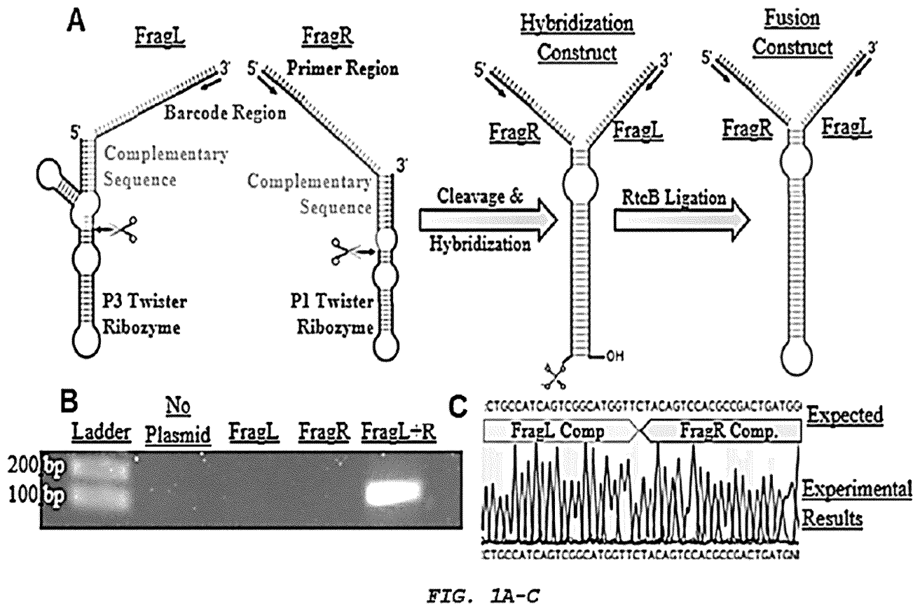

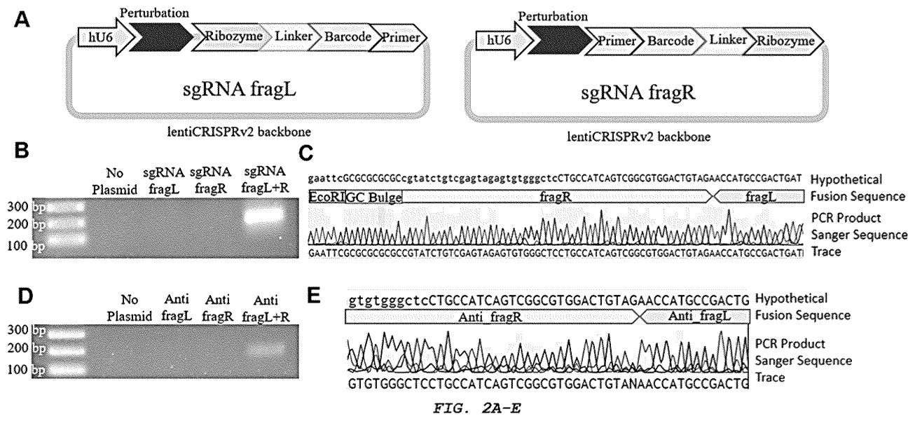

The disclosure describes the use of engineered RNA elements that undergo multiple endogenous processing events to create linked RNA fusion constructs that can be used for a variety of applications, including combinatorial genetic screens or inducible gene expression. These elements, termed the left fragment (fragL) and right fragment (fragR), have been engineered to be expressed from both polymerase II and polymerase III promoters. In a particular embodiment, the RNA elements can further comprise flanking amplification primers, and/or a variable barcode region. The RNA elements comprise a 45-base pair (bp) complementary region to one another and are tethered to the P3 and P1 self-cleaving Twister ribozymes, as illustrated in FIG. 1A. Upon transcription, the P3 Twister ribozyme self-cleaves and generates a 5′ hydroxyl group on the fragL construct while the P1 Twister ribozyme generates a 2′,3′-cyclic phosphate group on the 3′ end of the fragR construct. Due to the complementary regions of the fragments, the two transcripts will then hybridize, juxtaposing these two ends which enables recognition by the endogenous ligase, RtcB, creating a linked fusion construct. By use of endogenous processing of these constructs, there is no need for additional factors to be added. Linking two disparate library elements delivered to cells individually, but to be analyzed and fused together downstream, has great utility for a variety of applications. Furthermore, the Twister ribozymes undergo autocatalytic self-cleavage rapidly upon transcription, within tens of seconds to minutes, and the linked constructs have longer half-lives than the individual transcripts, making this approach robust across multiple time scales.

The choice of fusion constructs is completely tunable, allowing for additional classes of ribozymes or transfer RNAs to be used in combination with different ligases and RNA processing enzymes to create higher order fusion linkages.

The feasibility of this approach is demonstrated through a simple plasmid transfection experiment in which fragL and fragR were cloned into the lentiCRISPRv2 backbone downstream of the U6 promoter. These were delivered to cells either individually or in combination and RNA was isolated 48 hours later. Reverse transcription polymerase chain reaction (RT-PCR) was performed using the flanking amplification primers. As shown in FIG. 1B, the generated RNA fusion was only observed when both plasmids were transfected together. The PCR product was then purified, and Sanger sequenced to confirm the expected fusion sequence (FIG. 1C).

A major limitation with prior implementation of genetic interaction screens is the need to physically link multiple perturbations on the same library element in order to enable genotype to phenotype mapping. This can make library generation complex and prevents different classes of genome and transcriptome engineering toolsets from being readily combined.

The disclosure described the engineering of RNA-fusion constructs to enable genotype to phenotype linking at the RNA level so that multiple libraries (e.g. CRISPR-knockout, CRISPR activation/inhibition, open reading frame, shRNA, etc.) can be delivered to cells individually, but read out together. This approach is demonstrated by cloning the ribozyme-RNA fusion constructs downstream of the U6-driven sgRNA sequence in the lentiCRISPRv2 backbone (termed sgRNA fragL and sgRNA fragR) (FIG. 2A). As shown in FIG. 2B, the presence of a non-targeting sgRNA transcript in the 5′ position of the RNA-fusion constructs does not alter the RNA-processing machinery that create the ligated fusion. Furthermore, the EcoRI restriction site on the sgRNA fragR construct shown in FIG. 2C highlights the ability to identify a theoretical barcode placed in this location for perturbation mapping using RNA-sequencing.

The ribozyme-mediated RNA-fusion constructs of the disclosure allow for genotype to phenotype linking at the RNA level so that multiple libraries (e.g., CRISPR-knockout, CRISPR activation/inhibition, open reading frame, shRNA, etc.) can be delivered to cells individually, but read out together. By using this approach with the ribozyme-mediated RNA fusion constructs of the disclosure, genetic interactions screens can be highly multiplexed. This approach allows for the linking of disparate libraries such as CRISPR-knockout sgRNA libraries and open reading frame overexpression libraries. Further, unprecedented insight into the regulatory networks that dictate cell function can be realized using the RNA fusion constructs disclosed herein by using screens looking at the combined activation and inhibition of genes, through systems such as CRISPRactivation and CRISPRinhibition, in a cell. A screening strategy with RNA fusions is not limited to fitness measurements and can be coupled to screens using other readouts such as phenotype, protein expression, or other functional endpoints that are broadly applicable across the biological sciences.

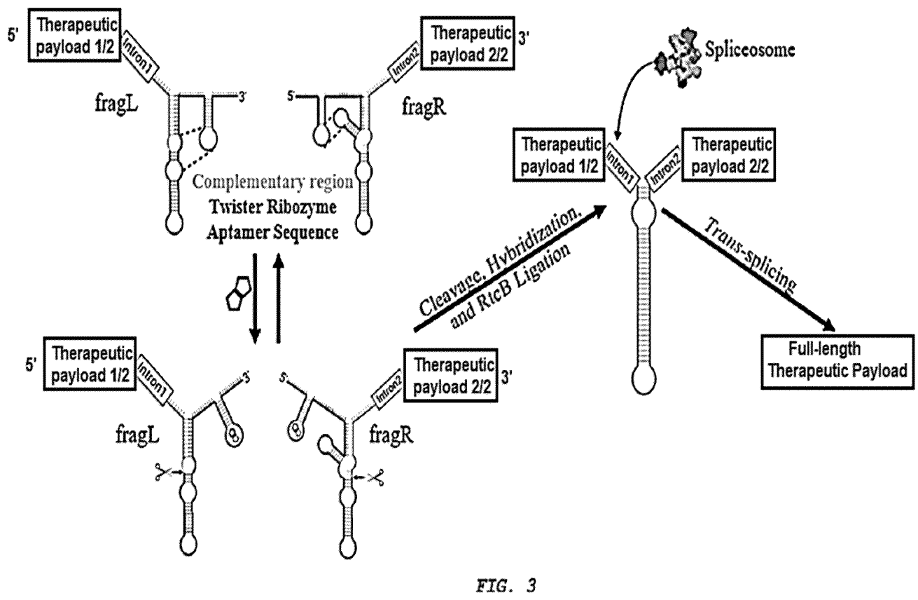

The RNA fusion constructs of the disclosure can be used in inducible gene expression systems. Inducible gene expression systems are powerful tools for a broad variety of basic and applied research areas, including functional genomics, tissue engineering, biopharmaceutical protein production, and gene therapy. The most common of these systems such as tetracycline-controlled operons, protein-protein interaction chimeric systems, and tamoxifen-controlled recombinase systems all require the addition of exogenous proteins. This can lead to immunogenic reactions in vivo and the delivery and transfection of these large-sized plasmids can be burdensome. The ribozyme-mediated RNA-fusion approach with intronic sequences described herein has broad utility. Through the addition of an aptazyme on either fragment that is responsive to molecules, such as tetracyline, theophylline, or guanine, the system of the disclosure can enable robust control of gene expression (see FIG. 3).

The RNA fusion constructs of the disclosure have great utility in gene therapy space to treat widespread diseases. In both type 1 and type 2 diabetes, insulin production is limited and therefore patients commonly must exogenously administer insulin when their blood glucose levels rise. The inducible ribozyme-mediated RNA-fusion system described herein can be adapted to contain two halves of the insulin gene fused to intronic sequences. The two constructs are constitutively present in muscular tissue, but one half would only be transcribed upon additional of an aptamer-binding ligand such as a synthetic sugar. This would lead to the rapid upregulation of ribozyme-mediated hybridization and splicing to generate the full length, functional insulin protein. Upon degradation of the inducer, the one fusion fragment would become repressed and no more insulin would be produced until more of the ligand is administered, thus replacing the need for painful and burdensome exogenous administration of insulin with an endogenous system with precise temporal control.

The inducible ribozyme-mediated RNA-fusion system described herein can be applied to generate an inducible gene expression system for the clotting factor IX for patients with hemophilia, the cystic fibrosis transmembrane conductance regulator protein for patients with cystic fibrosis, and the dystrophin protein for patients with Duchenne's muscular dystrophy. Broadly, any disease that results from a poorly expressed or mutated protein could benefit from the inducible ribozyme-mediated RNA-fusion system disclosed herein. This includes, but is not limited to, disease such as β-thalassemia, severe combined immunodeficiency, spinal muscle atrophy, and age-related macular degeneration.

The inducible ribozyme-mediated RNA-fusion system described herein can be broadly applied to gene therapies using the CRISPR/Cas toolset. CRISPR/Cas genome editing is highly adaptable and has been engineered to investigate and treat genetic diseases, cancers, immunological diseases, and infectious diseases. A major limitation in the translation of these therapies is the inability to control the expression of the Cas protein in vivo. The inducible ribozyme-mediated RNA-fusion system described herein can overcome this limitation by fusing two portions of the Cas protein to intronic sequences in the fragL and fragR constructs. One of these would be under the control of an inducer as described herein, making the expression of the Cas protein and its subsequent function completely inducible. This would enable precise control over the genome editing that is mediated by the CRISPR/Cas system. It is not limited to gene knockouts and could be broadly adapted to aid in controlled and inducible non-homologous end joining, homology directed repair, single-base exchanges, transcriptional regulation, base editors, PRIME editors, and RNA editing.

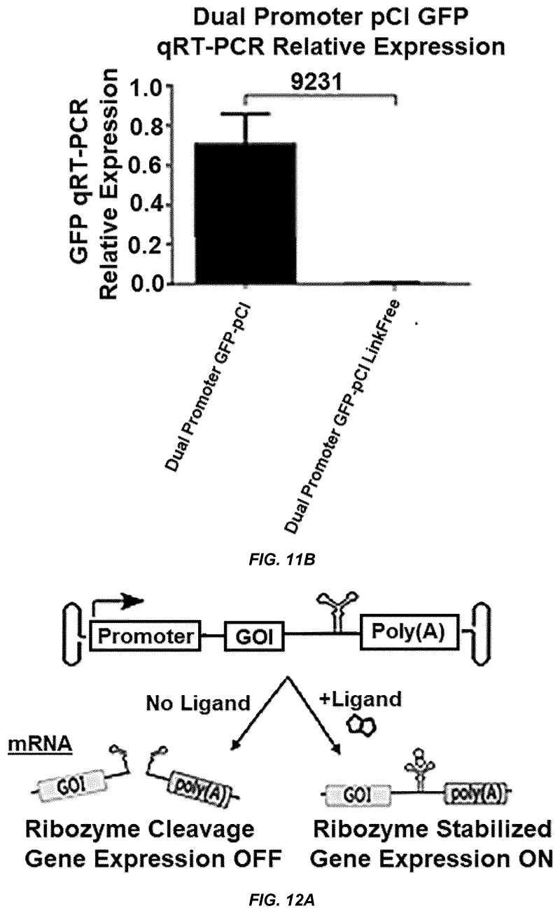

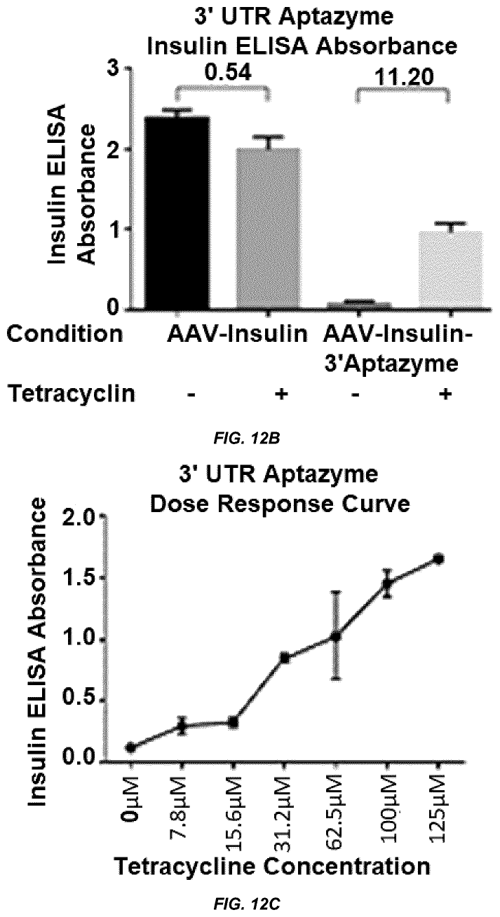

Additionally, experiments can be performed utilizing this system (see, e.g., FIG. 12A) to control the expression of the Yamanaka factors, including Oct3/4, Sox2, Klf4, and c-Myc (OSKM), in vivo. OSK and c-Myc can be cloned into an AAV expression vector with the aptazyme of choice subsequently cloned into the 3′ UTR. The Oct3/4, Sox2, and Klf4 can be cloned into a polycistronic vector and separated by the self-cleaving 2A peptides. c-Myc, a known oncogene, can be cloned into another plasmid with its own aptazyme control element. These plasmids can then be packaged into AAV vectors and then delivered either separately or together based on the reprogramming application at hand.

As the ribozyme rapidly cleaves the 3′-poly(A) tail upon transcription, the background expression of OSKM will be low and transduction via iAAV will not alter cellular state at baseline expression. Upon delivery of the ligand specific for the aptamer, the ribozyme will stabilize and the transduced cells will exhibit higher expression levels of OSKM based on the dose of the ligand that was delivered. With the rapid turnover kinetics of mRNA transcripts, the stabilization of the ribozyme and resulting gene expression levels are directly dependent on the half-life of the ligand delivered and the administration regimen that was chosen, thus enabling dynamic, pulsatile, and transient control of OSKM expression. As these transcription factors have been thoroughly studied to induce a state of pluripotency based upon their expression levels. This system can be utilized to dynamically reprogram cells in vivo.

Additionally, there have been a number of other “reprogramming factors” which have been implicated in directing cellular phenotype via their overexpression. Like OSKM, these factors require temporal control to effectively, and safely exhibit their effect on cell state. Therefore, the ribozyme-mediated control system can be used to dynamically control the expression of a broad range of genes in vivo which have an ability to reprogram cellular identity. These genes which the system could be applied to are included, but not limited to, those genes listed in Table 1.

This system is further tunable as the AAV serotype used can be altered without having to alter the expression plasmid. Various serotypes can be used which specifically target tissues such as AAV8 for the liver, AAV9 for skeletal muscle, or AAV-PHP.B for the central nervous system. Furthermore, engineered recombinant AAVs which specifically target distinct cell types can also be utilized in addition to the broad range of serotypes already available to further enhance the specificity of the partial reprogramming system.

TABLE 1

| GENE | ROLE (Involved in) | GENE | ROLE (Involved in) |
|---|---|---|---|
| ASCL1 | neuronal specification & differentiation. | LMX1A | neural develop. |
| | Demonstrated to drive neuronal | | |
| | differentiation from hPSCs | | |
| ASCL3 | salivary gland cell develop. | MEF2C | cardiac develop. |
| ASCL4 | develop. of skin | MESP1 | cardiac develop. |
| ASCL5 | Paralog of ASCL4 | MITF | pigment cell & melanocyte differentiat. |
| ATF7 | early cell signaling, binds cAMP response | MYC | cell proliferation, differ. & apoptosis. |
| | element | | Reprogramming factor for induction of pluripotency |
| CDX2 | trophectoderm specification & | MYCL | cell proliferation, differentiation & apoptosis |
| | differentiation | | |
| CRX | photoreceptor differentiation | MYCN | cell proliferation & differentiation |
| ERG | endothelial cell specification & | MYOD1 | skeletal muscle specification & differentiation, |
| | differentiation | | demonstrated to induce differentiation of hPSCs to |
| | | | skeletal muscle |
| ESRRG | cardiac develop. | MYOG | skeletal muscle specification & diff. |
| ETV2 | haemato-endothelial specification & diff. | NEURO | neuronal specification & diff .. Demonstrated to |
| | & vasculogenesis | D1 | induce neuronal diff. in hPSCs |
| FLI1 | haemato-endothelial specification & | NEURO | neuronal specification & differentiation |
| | differentiation | G1 | |
| FOXA1 | branching morphogenesis, develop. of | NEURO | pancreatic develop. & neuronal specification & |
| | lung, liver, prostate, pancreas | G3 | differentiation |
| FOXA2 | branching morphogenesis, develop. of | NRL | photoreceptor develop. |
| | notochord, lung, liver, prostate & | | |
| | pancreas | | |
| FOXA3 | cell glucose homeostasis | ONE- | retinal, liver, gallbladder & pancreatic develop. |
| | | CUT1 | |
| FOXP1 | develop. of hematopoietic cells, lung & | OTX2 | photoreceptor differentiation, pineal gland develop. |
| | esophagus, & neuronal develop. | | & induction & specification of forebrain & midbrain |
| GATA1 | erythroid develop. | PAX7 | specification & differentiation of satellite ells, |
| | | | demonstrated to induce myogenic precursor |
| | | | differentiation in hPSCs |
| GATA2 | hematopoietic develop. | POUIF1 | pituitary gland develop. |
| GATA4 | cardio. develop. | POU5F1 | regulation of pluripot & embryogenesis. |
| | | | Reprogramming factor for induction |
| GATA6 | cardiac, lung, endoderm & | RUNX1 | hematopoietic cell develop. |
| | extraembryonic develop. | | |
| GLI1 | neural stem cells prolif & neural tube | SIX1 | kidney, ear & olfactory epithelium develop. |
| | develop. | | |
| HAND2 | cardiac develop. | SIX2 | kidney develop. |
| HNF1A | liver, kidney, pancreatic & gut develop. | SNAJ2 | neural crest develop., epithelial-mesenchyme |
| | | | transition & melanocyte stemcell develop. |
| HNF1B | liver, kidney, pancreatic & gut develop. | SOX10 | neural crest & neuronal develop. |
| HNF4A | liver, kidney, pancreatic & gut develop. | SOX2 | regul. of pluripot & embryogenesis & in neuronal |
| | | | develop. Reprogramming factor for induction of |
| | | | pluripotency |
| HOXA1 | neural & cardio develop. | SOX3 | neuronal & pituitary develop. |
| HOXA10 | fertility, embryo viability, & regulation of | SPI1 | hematopoietic cell develop. |
| | hematopoietic lineage commitment | | |
| HOXA11 | kidney develop. | SPIB | differentiation of lymphoid cells |
| HOXB6 | lunch & epidermal develop. | SPIC | macrophage develop. |
| KLF4 | regulation of pluripotency & develop. of | SRY | sex determination & spermatogenesis |
| | skin, Reprogramming factor for induction | | |
| | of pluripotency | | |
| LHX3 | pituitary gland develop. | TBX5 | cardiac develop. |
| | | TFAP2C | trophectoderm develop. |

Utilizing the system described herein for the in vivo control of reprogramming factors, the system can be harnessed for a broad range of applications.

Generally, transient expression of the Yamanaka factors in vivo has been demonstrated to ameliorate aging hallmarks. The system of the disclosure with OSKM and the 3′-UTR aptazyme could be packaged into an AAV, designed to either have broad tropism across the body, or targeted to a specific organ via an engineered AAV. This could then be administered to the subject and allowed to transduce its target organs for a short period of time. Subsequently, the ligand that is specific for the aptamer sequence could be administered at the desired dose and treatment regimen in order to achieve cyclic expression of OSKM. The physiological alterations induced by this approach could include a reduction in the DNA damage response associated with aging, downregulation of senescence and stress-related genes, and alterations to the epigenetic modifications that occur with aging. These molecular alterations at the cellular level have important implications for reducing the systematic aging issues. Furthermore, in the context of specific diseases related to aging, such as Hutchinson-Gilford Progeria syndrome, this strategy can be an important therapeutic option to systematically reduce physiological hallmarks of aging while also prolonging the lifespan of those affected.

On the tissue-specific level, the system of the disclosure can demonstrate an important therapeutic benefit as engineering of the AAV capsid can be utilized for cell-specific targeting of the inducible-reprogramming strategy. In the central nervous system, transient expression of OSK could be utilized to restore youthful DNA methylation patterns and transcriptomes in the retinal ganglion cells in order to promote axonal regeneration after injury and promote vision restoration for the aging population or those afflicted with visual impairments such as glaucoma. Similarly, targeting the system of the disclosure to specific brain regions (e.g., hippocampus) can be an important tool for improving memory through specific targeting of dentate gyrus cells. In the cardiovascular system, targeting the system of the disclosure to cardiomyocytes can lead to dedifferentiation of these post-mitotic cells. This enabled regenerative capacity has the potential to broadly improve cardiac function with the potential to greatly improve cardiomyocyte recovery following traumatic events such as myocardial infarction. Administration of the inducible OSKM construct as described herein and then treating the afflicted individual with the inducing ligand could drastically improve recovery from cardiovascular events. Furthermore, myofiber- and liver-specific transient expression mediated by the system of the disclosure has the ability to promote muscle regeneration in vivo, which has broad implications in both the aging and diseased setting.

Aside from induced AAV-aptazyme mediated expression of OSKM, the methods and compositions of the disclosure can be applied to other reprogramming transcription factors (TFs) as well (Table 1). Depending on the outcome desired, TFs could be delivered either individually or in combination with either matching aptazyme sequences or separate aptazymes to enable temporal control of gene expression. These engineered TFs can be applied to the healthy and diseased settings with even broader implications for the whole field of regenerative medicine. The iAAV-partial reprogramming approach of the disclosure has broad applications across a diverse array of organ systems and disease settings.

RNA is inherently transient and this transience impacts their activity both as an interacting moiety as well as a template. Circularization of RNA improve persistence, however simple and scalable approaches to achieve the same are lacking. Utilizing autocatalytic RNA circularization as described herein, the disclosure provides compositions and methods of in situ circularized RNAs (icRNAs) for durable protein translation. Specifically, an in vitro transcribed linear RNA that bears an internal ribosome entry site coupled to a messenger RNA of interest that is in turn flanked by ribozymes is provided. Delivery of these linear RNAs into cells yields in situ circularized molecules upon autocatalytic cleavage of the ribozymes that leave termini which are ligated by endogenous RNA ligases (e.g., the ubiquitous endogenous RNA ligase RtcB) (see, e.g., FIG. 15). This scalable icRNA system has broad utility in basic science and therapeutic applications.

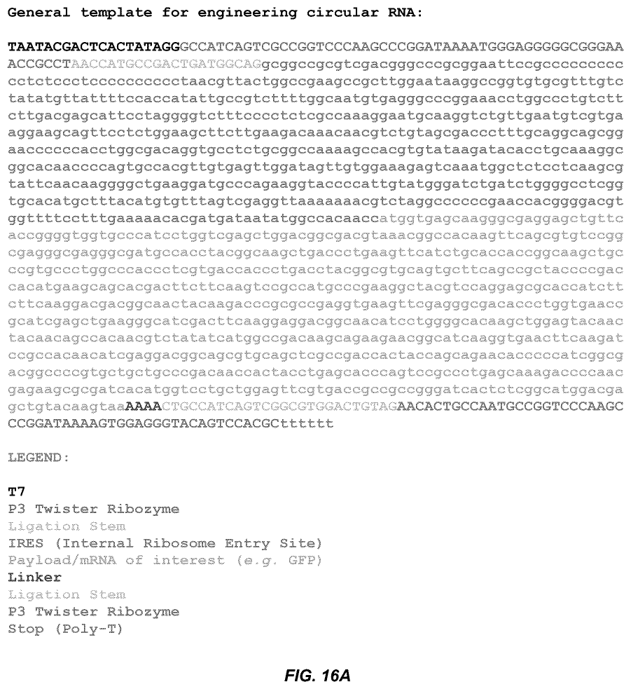

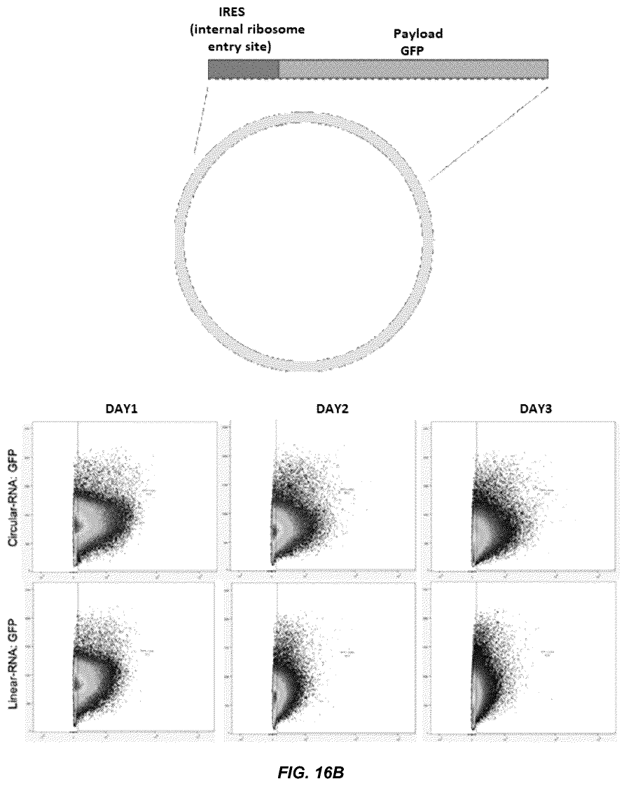

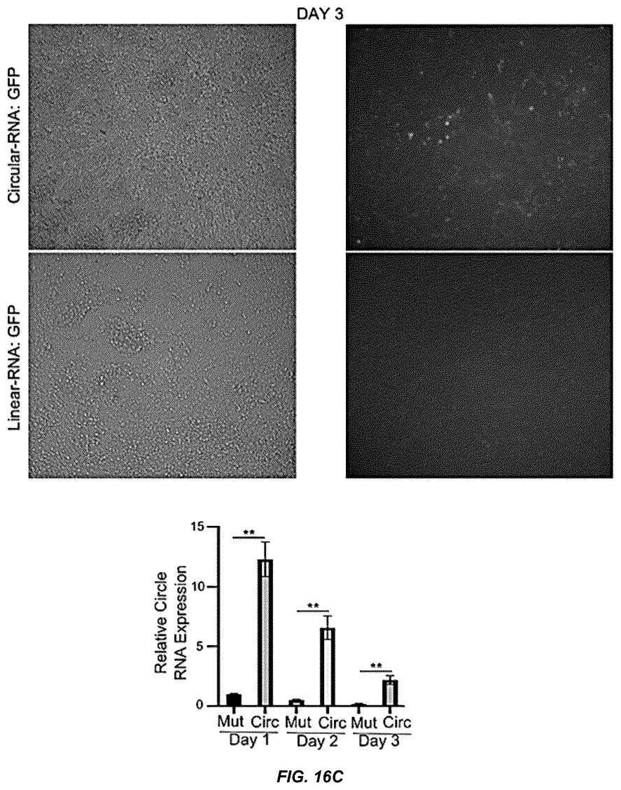

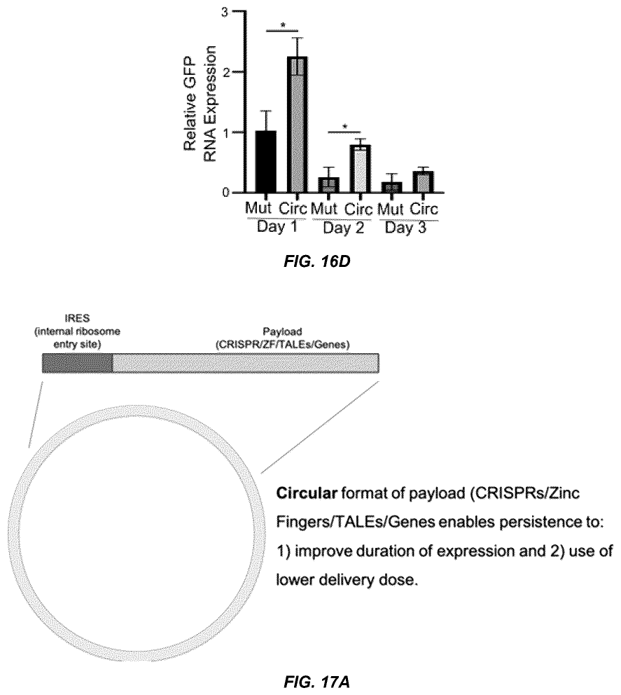

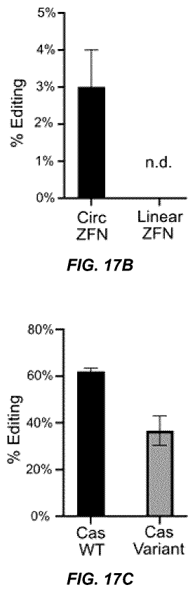

The icRNA system is exemplified herein in three contexts: first, durable protein expression via this system using GFP as a test protein (FIG. 16A-D); second, improved genome targeting via zinc finger nucleases; and third, genome targeting via CRISPRs, including deimmunized Cas9 proteins (FIG. 17A-C).

Compositions herein can be used to treat a disease or condition in a subject. For example, a ribozyme-activated RNA construct of the disclosure can be administered to treat a disease described herein.

A pharmaceutical composition can comprise a first active ingredient. The first active ingredient can comprise a ribozyme-activated RNA construct of the disclosure. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient.

A composition described herein can compromise an excipient. In some cases, an excipient can comprise a pharmaceutically acceptable excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some cases, a carrier or a diluent can comprise an excipient. In some cases, a carrier or diluent can comprise a water, a salt solution (e.g., a saline), an alcohol or any combination thereof.

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some cases, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some cases, a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some cases, a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some cases, a pharmaceutical formulation can comprise a disintegrant as an excipient. In some cases, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some cases, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some cases, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some cases, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

A composition may comprise a combination of the active agent, e.g., a ribozyme-activated RNA construct of the disclosure, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

In some embodiments, a pharmaceutical composition can be formulated in milligrams (mg), milligram per kilogram (mg/kg), copy number, or number of molecules. In some cases, a composition can comprise about 0.01 mg to about 2000 mg of the active agent. In some cases, a composition can comprise about: 0.01 mg, 0.1 mg, 1 mg, 10 mg, 100 mg, 500 mg, 1000 mg, 1500 mg, or about 2000 mg of the active agent.

A subject, host, individual, and patient may be used interchangeably herein to refer to any organism eukaryotic or prokaryotic. In some cases, subject may refer to an animal, such as a mammal. A mammal can be administered a ribozyme-activated RNA construct of the disclosure or composition as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder. In other embodiments, a subject has or is suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

In some embodiments, method of treating a human in need thereof can comprise administering to the human a ribozyme-activated RNA construct of the disclosure. In some embodiments, compositions herein can be used to treat disease and conditions. A disease or condition can comprise a neurodegenerative disease, a muscular disorder, a metabolic disorder, an ocular disorder, or any combination thereof. The disease or condition can comprise cystic fibrosis, albinism, alpha-1-antitrypsin deficiency, Alzheimer disease, Amyotrophic lateral sclerosis (ALS), Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCA1 and 2 linked breast cancer and ovarian cancer). In some cases, a disease or condition can comprise Mucopoysaccharidosis type I (MPSI). In some cases, the MPSI can comprise Hurler syndrome, Hurler-Scheie syndrome, Scheie syndrome, or any combination thereof. The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof. Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition may comprise a muscular dystrophy. A muscular dystrophy may include myotonic, Duchenne, Becker, Limb-girdle, facioscapulohumeral, congenital, oculopharyngeal, distal, Emery-Dreifuss, or any combination thereof. A disease or condition may comprise pain, such as a chronic pain. Pain may include neuropathic pain, nociceptive pain, or a combination thereof. Nociceptive pain may include visceral pain, somatic pain, or a combination thereof.

A vector can be employed to deliver a ribozyme-activated RNA construct of the disclosure. A vector can comprise DNA, such as double stranded DNA or single stranded DNA. A vector can comprise RNA. In some cases, the RNA can comprise one or more base modifications. The vector can comprise a recombinant vector. In some cases, the vector can be a vector that is modified from a naturally occurring vector. The vector can comprise at least a portion of a non-naturally occurring vector. Any vector can be utilized. In some cases, the vector can comprise a viral vector, a liposome, a nanoparticle, an exosome, an extracellular vesicle, or any combination thereof. In some embodiments, plasmid vectors can be prepared from commercially available vectors. In other embodiments, viral vectors can be produced from baculoviruses, retroviruses, adenoviruses, AAVs, or a combination thereof. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves. Alphavirus vectors, such as Semliki Forest virus-based vectors and Sindbis virus-based vectors, have also been developed for use in gene therapy and immunotherapy. In aspects where gene transfer is mediated by a retroviral vector, a vector construct can refer to the polynucleotide comprising the retroviral genome or part thereof, and a gene of interest. In some cases, a vector can contain both a promoter and a cloning site into which a polynucleotide can be operatively linked. Such vectors are capable of transcribing RNA in vitro or in vivo and are commercially available. In some cases, a viral vector can comprise an adenoviral vector, an adeno-associated viral vector (AAV), a lentiviral vector, a retroviral vector, a portion of any of these, or any combination thereof. In some cases, a nanoparticle vector can comprise a polymeric-based nanoparticle, an aminolipid based nanoparticle, a metallic nanoparticle (such as gold-based nanoparticle), a portion of any of these, or any combination thereof. In some cases, a vector can comprise an AAV vector. A vector can be modified to include a modified VP1 protein (such as an AAV vector modified to include a VP1 protein). An AAV can comprise a serotype-such as an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, a derivative of any of these, or any combination thereof.

In some embodiments, a vector can comprise a nucleic acid that encodes a linear precursor of a ribozyme-activated RNA construct of the disclosure. In some embodiments, a nucleic acid can comprise a linear precursor of a ribozyme-activated RNA construct of the disclosure. In some cases, the nucleic acid can be double stranded. In some instances, the nucleic acid can be DNA or RNA. In some cases, a nucleic acid can comprise more than one copy of a ribozyme-activated RNA construct of the disclosure. For example, a nucleic acid can comprise 2, 3, 4, 5, or more copies of a ribozyme-activated RNA construct of the disclosure. In some instances, the nucleic acid can comprise a U6 promoter, a CMV promotor or any combination thereof.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5’ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant coding regions in the desired host cell are numerous and familiar to those skilled in the art. Virtually any promoter capable of driving these genetic elements is suitable for use in the disclosure. For example, a pol III promoter, a U6 promoter, a CMV promoter, a T7 promoter, an H1 promoter, can be used to drive expression. Termination control regions may also be derived from various genes native to the preferred hosts.

Administration of a ribozyme-activated RNA construct of the disclosure can be effected in one dose, continuously or intermittently throughout the course of treatment. Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can vary and depend on the disease or condition. Routes of administration can vary with the composition used for treatment, the purpose of the treatment, the health condition or disease stage of the subject being treated, and target cell or tissue. Non-limiting examples of routes of administration include oral administration, nasal administration, injection, and topical application.

Administration can refer to methods that can be used to enable delivery of compounds or compositions to the desired site of biological action (such as DNA constructs, viral vectors, or others). These methods can include topical administration (such as a lotion, a cream, an ointment) to an external surface of a surface, such as a skin. These methods can include parenteral administration (including intravenous, subcutaneous, intrathecal, intraperitoneal, intramuscular, intravascular or infusion), oral administration, inhalation administration, intraduodenal administration, and rectal administration. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition.

Administration or application of a composition disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of compositions disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered or applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

Kits and articles of manufacture are also described herein that contain ribozyme-mediated RNA-fusion constructs or inducible ribozyme-mediated RNA-fusion system described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more RNA fusion constructs described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### Examples

Generation of RNA fusion constructs using plasmids. A simple plasmid transfection experiment was performed where fragL and fragR were cloned into the lentiCRISPRv2 backbone downstream of the U6 promoter. These were delivered to cells either individually or in combination and RNA was isolated 48 hours later. Reverse transcription polymerase chain reaction (RT-PCR) was performed using the flanking amplification primers. As shown in FIG. 1B, RNA fusion was observed when both plasmids were transfected together and not singly. The PCR product was then purified, and Sanger sequenced to confirm the expected fusion sequence (see FIG. 1C).

RNA-fusion constructs with small RNAs and polymerase III promoters Polymerase III (pol-III) promoters. The ribozyme-RNA fusion constructs were cloned downstream of the U6-driven sgRNA sequence in the lentiCRISPRv2 backbone (termed sgRNA fragL and sgRNA fragR) (see FIG. 2A). As shown in FIG. 2B, the presence of a non-targeting sgRNA transcript in the 5′ position of the RNA-fusion constructs did not alter the RNA-processing machinery that created the ligated fusion. Furthermore, the EcoRI restriction site on the sgRNA fragR construct shown in FIG. 2C highlights the ability to identify a theoretical barcode placed in this location for perturbation mapping using RNA-sequencing.

RNA-fusion constructs for combinatorial screening. The constructs were designed to be amenable to combinatorial screening by orienting the fragL and fragR sequences to be in the antisense direction to the lentiviral 5′ long terminal repeat promoter-controlled transcript (termed anti fragL and anti fragR). This design prevents degradation of transcripts during lentiviral production due to the fast cleavage rates of the Twister ribozyme implemented in the system. Correct ligation RNA-fusion construct sequences in the antisense direction were confirmed by RT-PCR and Sanger sequencing (see FIG. 2D-E). Furthermore, anti fragL and anti fragR constructs were cloned into plasmids containing fluorescent reporters to enable rapid selection of cells in which RNA-fusions are generated. Cells receiving both plasmids are easily identified via fluorescence and can undergo fluorescence-activated cell sorting to screen only those cells receiving both fragments in which the RNA fusion construct can be generated.

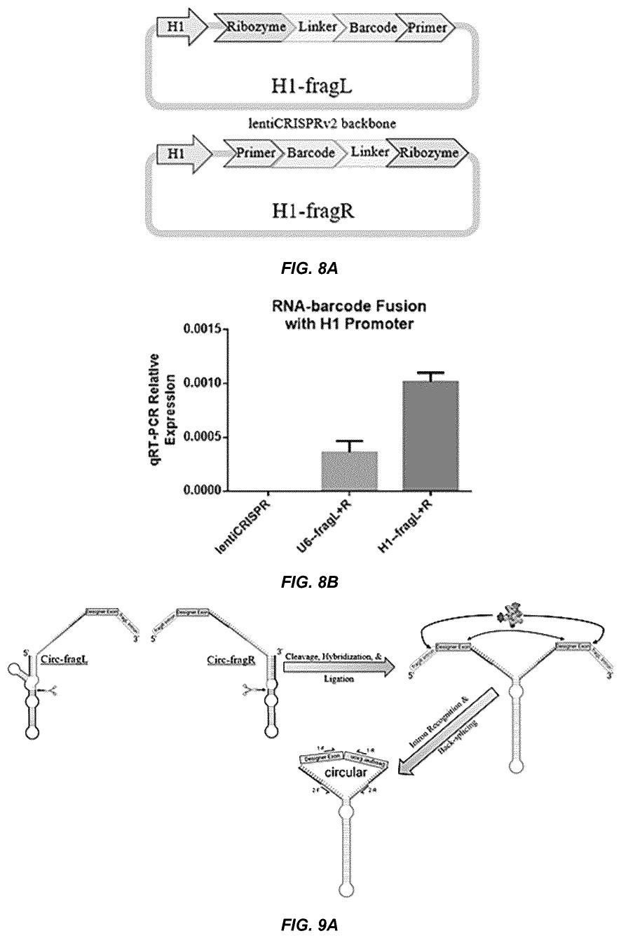

As it is desirable that the RNA-fusion system is modular across various polymerase III promoters, the constructs are demonstrated to be capable of fusing with one another when driven by the H1 promoter (FIG. 8A), which has been shown to demonstrate both polymerase II and polymerase III activity. As shown in FIG. 8B, when driven by the H1 promoter, the presence of the RNA fusion construct can be detected. This shows the system is flexible and amenable to the different promoters that may be present on various genome engineering constructs of interest.

Use of RNA-fusion constructs for combinatorial screening. By use of EcoRI restriction sites (se FIG. 2C), variable barcodes can be cloned into each sgRNA, or other perturbation, so that the constructs can be identified by the barcodes. This will ensure that each cell in the screen receives a unique pair of barcodes, that serves as unique molecular identifiers (UMIs), and in downstream counting all UMIs will be collapsed. The library further undergoes next generation sequencing (NGS) to create a lookup table matching barcodes to perturbations. This library is transduced at an intermediate multiplicity of infection such that most cells receive 2 library elements. Cells are then be sorted for GFP/tdTomato double positive fluorescence to screen cells receiving two barcodes. Cells are collected, and RNA isolated at multiple timepoints in the screen (day 3, day 14, day 21, and day 28) to ensure robust fitness measurements are obtained. The UMIs from the harvested RNAs are then selectively reverse transcribed, amplified, and sequenced. Next, the identified barcodes at each timepoint are mapped back to the initially generated lookup table to map genotype to phenotype across the experiment.

Ribozyme-mediated RNA fusion with larger RNAs and polymerase II promoters. To further probe the broad-range utility of ribozyme-mediate RNA fusion approach, fusion expression driven by a polymerase II (pol-II) promoter was next investigated. RNA polymerase II is responsible for the transcription of the most cellular genes (mRNA) and pol-II promoters such as EF1α, SV40, CMV, and RSV, as well as tissue specific promoters such as NEUROD2 in the CNS and TBX20 in the aorta, are important for the translation of effective gene therapies. It was postulated that the ribozyme-mediated RNA fusion approach could be engineered into an AAV backbone to enable precise control over the expression of various gene therapeutic modalities. The overall schematic for this approach is outlined in FIG. 3. Ligand-responsive aptamer sequences are coupled to the 3′ end of the fragL and 5′ end of the fragR construct. These aptamers have tertiary interactions with the ribozyme, stabilizing the catalytic loop such that no self-cleavage takes place. In the absence of cleavage, two fragments never hybridize and a ligated RNA-fusion transcript is not generated. Addition of a ligand disrupts the structural conformation of the aptamer so that it no longer stabilizes the ribozyme. The ribozyme then undergoes self-cleavage, generating the unique ends, and allowing the complementary regions of the two fragments to hybridize to one another and then by ligated together by the RtcB ligase.

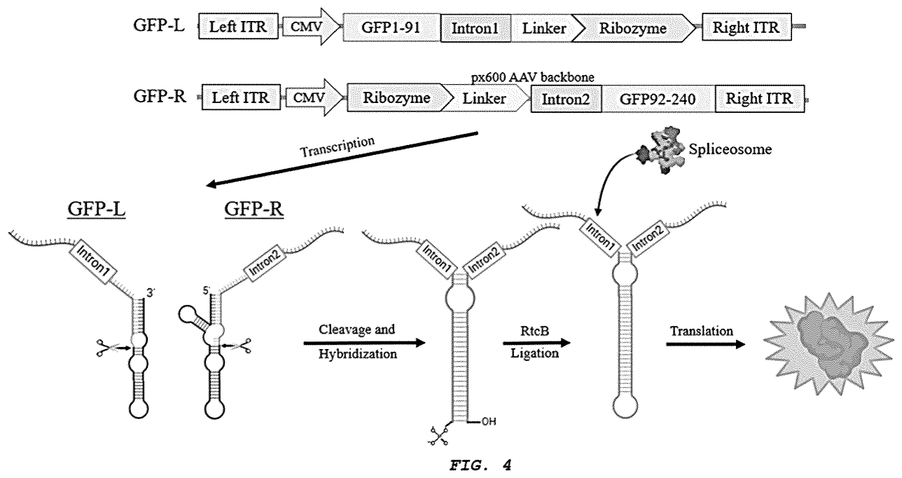

To enable the inducible system to generate a full-length transcript and subsequent protein with no additional features, intronic sequences were fused between the therapeutic payload and the ribozyme-mediated fusion constructs (see FIG. 3). Once the two fragments hybridize and are ligated together, the features of the two introns, such as the 5′ and 3′ splice sites, the branch point, and the polypyrimidine tract can be recognized by cellular splicing machinery. Trans-splicing then takes place to remove the intronic sequences and fuse the two exons together to generate a full-length, functional transcript. The outline for the concept is shown in FIG. 4. Two halves of green fluorescent protein (GFP-L and GFP-R) that do not fluoresce when expressed individually were conjugated to intronic sequences. The ribozyme-mediated RNA fusion constructs were then cloned into the px600 AAV backbone. A two-intron model was initially utilized with longer intronic sequences (>250 bp each) derived from the Dihydrofolate reductase (DHFR) gene, which have previously been shown to undergo efficient splicing when linked together.

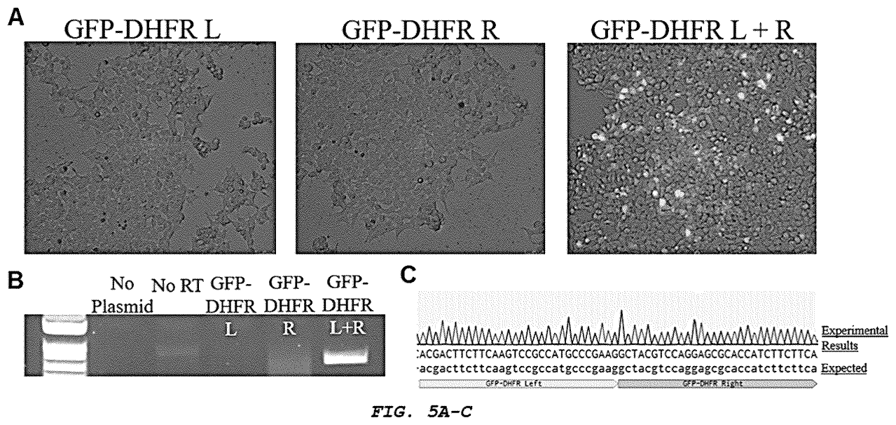

When each intron was cloned between one half of the GFP transcript and one construct of the ribozyme-mediated RNA fusion pair and transfected into HEK293T cells, fluorescence was only observed when both sequences were delivered in combination with one another (see FIG. 5A). RNA was isolated from these cells. RT-PCR and Sanger sequencing were used to show that the introns were efficiently spliced out and the full length GFP transcript was generated from the two halves (see FIG. 5B-C). The ability to generate a functional fluorescent protein through flow cytometry was further shown in FIG. 7.

Ribozyme-mediated exonuclease resistant and circular RNA fusion constructs. As this fusion barcode approach is mediated at the RNA-level, use of diverse RNA processing machinery can be used to further engineer the constructs to achieve desirable properties. The ribozyme mediated fusion barcodes can be engineered to contain a short “exonuclease-resistant” RNA (xrRNA) structures derived from viral genomes (FIG. 4). Once transcribed, these RNA elements can serve to prevent the processive exoribonucleolytic degradation of RNA. By integrating these into the fusion barcodes, the robustness and persistence of the barcode constructs can be increased in a cellular environment.

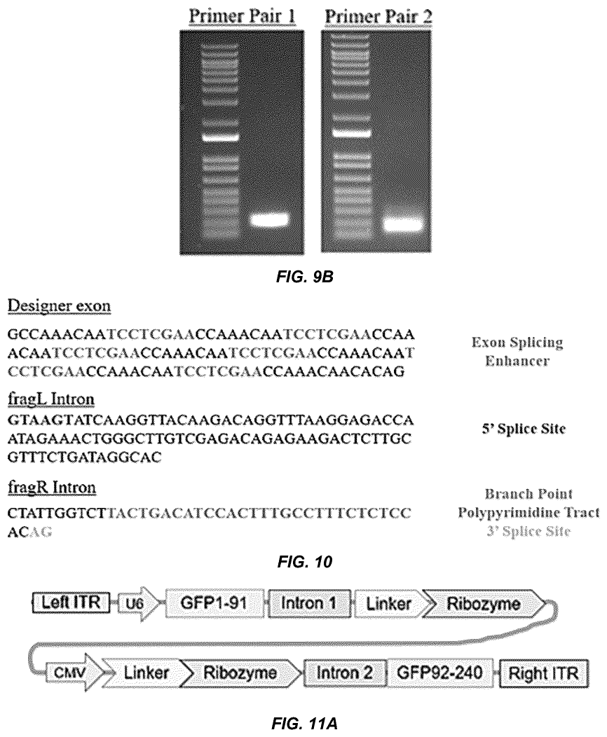

To further increase the persistence of the RNA barcodes in a cellular environment, a circularized RNA barcode design, which is illustrated in FIG. 9A. In this design, the left fragment design has been modified to contain a “designer exon” and a chimeric intron on the 3′ end of the construct (circ-fragL). The designer exon has can be optimized to be the appropriate length with various exon splicing enhancer (ESE) elements to promote the splicing out of the introns (sequences shown in FIG. 10). The right fragment is also modified on its 5′ end with an added intron followed by another designer exon and then the original fragR construct shown in FIG. 1 (circ-fragR). Once transcribed, the ribozyme can self-cleave allowing the complementary sequences to hybridize to one another. The ends generated by ribozyme cleavage can then be recognized by an endogenous RNA ligase, RtcB, which will create one fused end.

With the exons and introns close to one another, the spliceosome machinery will then recognize the various intronic elements and splice out the two introns, joining the two designer exons to one another via this back-splicing mechanism. This will result in the other end being fused together, creating a circularized construct. This construct circularizes in cells by transfecting HEK293T cells with the circ-fragL and circ-fragR. RNA was isolated 48 hours after transfection, reverse transcribed. PCR was performed to determine if circular RNA persisted. By utilizing two directional primer pairs, the RNA barcodes were being circularized and could be recovered from the pool of cellular RNA (FIG. 9B). With these constructs, the barcodes are robust in a complex cellular environment and therefore are better able to be used across a wider variety of screening platforms.

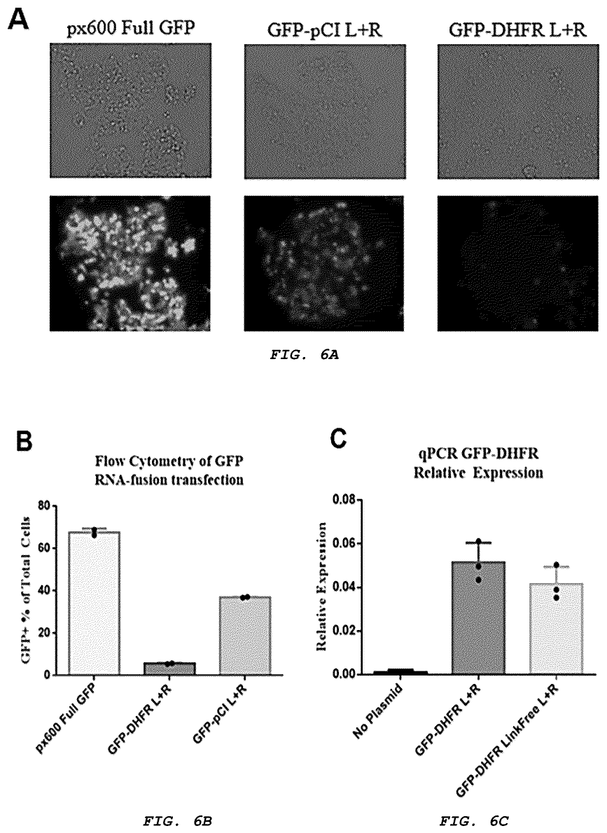

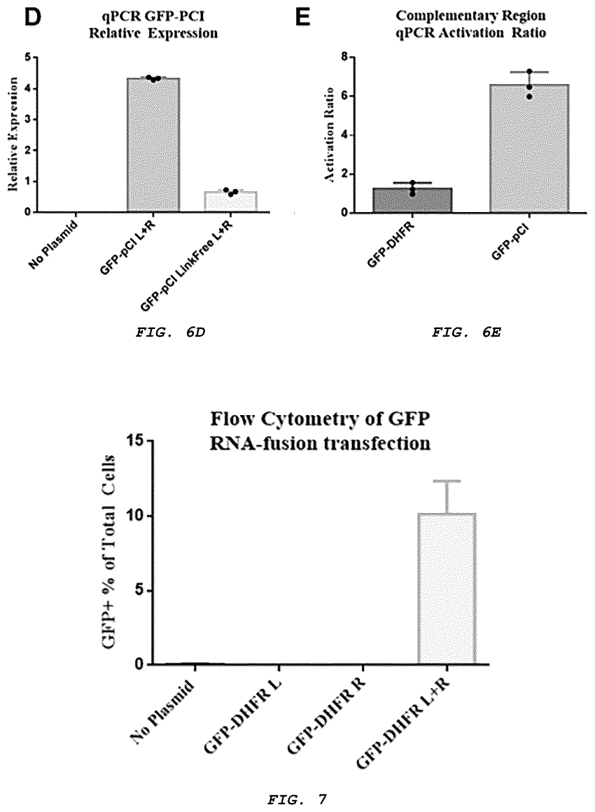

Use of a chimeric intron approach for gene expression. The gene expression system was tested by utilizing a chimeric intron (pCI) approach in which GFP-L was fused to the 5′ end of an intron derived from the β-hemoglobin gene containing the 5′ splice site while GFP-R was fused to the 3′ end of an intron derived from the human IgG gene containing the branch point, polypyrimidine tract, and 3′ splice site (FIG. 11). Using the pCI vectors containing the ribozyme-mediated RNA fusion constructs, robust expression of GFP was observed upon transfection of both plasmids. Fluorescence levels were comparable to the full length GFP transcript expressed individually, demonstrating a robust ability of the approach to generate functional protein (see FIG. 6A). When GFP total fluorescence was quantified with flow cytometry, the pCI system yielded 37% of cells GFP+, while the full GFP transcript in the px600 AAV backbone yielded 67% of cells GFP+ (see FIG. 6B), which is impressive considering each cell must be transfected with two plasmids in the pCI approach.

With high expression levels of GFP using the pCI system, the requirement that the complementary regions of the RNA from the fragL and fragR constructs hybridize with one another in order to get efficient splicing of the introns was next investigated. To investigate this, constructs were generated that only contained the intron and the GFP half, lacking the complementary region and the ribozyme (termed “LinkFree”). HEK293T cells were transfected using these constructs and their full-length counterparts. RNA was isolated after 48 hours, and RT-qPCR was performed to analyze GFP transcript relative expression normalized to GAPDH. As shown in FIG. 6C, GFP-DHFR intronic sequences were able to be spliced out without a hybridization requirement, as relative expression did not change much in the LinkFree system. Alternatively, the pCI system indicated a much stronger requirement for hybridization with an activation ratio (Full-length expression/LinkFree expression)>6 (see FIG. 6D-E).

For delivering various therapeutic modalities, it is sometimes advantageous to administer one viral vector to deliver the gene of interest. Therefore, constructs were further engineered with an inducible gene expression system to be contained on one plasmid so that the entire mechanism could be packaged into a single AAV vector. To accomplish this, a dual-promoter based system was used in which GFP-pCI-L was driven by the U6 promoter and GFP-PCI-R is driven by the CMV promoter (FIG. 11A). The transfection of this dual-promoter plasmid construct in HEK293T cells lead to expression of GEP (FIG. 11B) with an activation ratio (>80-fold) when compared to the dual-promoter link free design.

Accordingly, the RNA fusion concept utilizing ribozymes can be used to bring together separately delivered elements to generate a full length, functional proteins. Using pCI intron sequences, high expression levels were realized with a strong increase in expression upon hybridization of the ribozyme-generated complementary regions. This construct was further engineered to be packaged into one plasmid so that it could be packaged into an individual AAV capsid and delivered as a single vector. It is expected that controlled, inducible expression of an effector with the addition of a synthetic riboswitch or other inducible element on one or both constructs can also be utilized.

The disclosure demonstrates this feasibility of using a ribozyme-based approach including a tetracycline-responsive ribozyme embedded into the 3′ untranslated region (UTR) of a gene of interest (GOI). In this system, at the baseline level, the hammerhead ribozyme will self-cleave upon transcription, cleaving off the 3′-poly(A) tail, destabilizing the RNA transcript and leading to relatively low gene expression. Addition of the tetracycline ligand leads to the ligand binding to the tetracycline-responsive aptamer with a high affinity. This induces a conformational change in the secondary structure of the RNA molecule which disrupts the tertiary loop-loop interaction of the hammerhead ribozyme which prevents cleavage of the poly(A) tail and stabilizes the mRNA transcript, allowing for increased expression of the GOI (FIG. 12A).

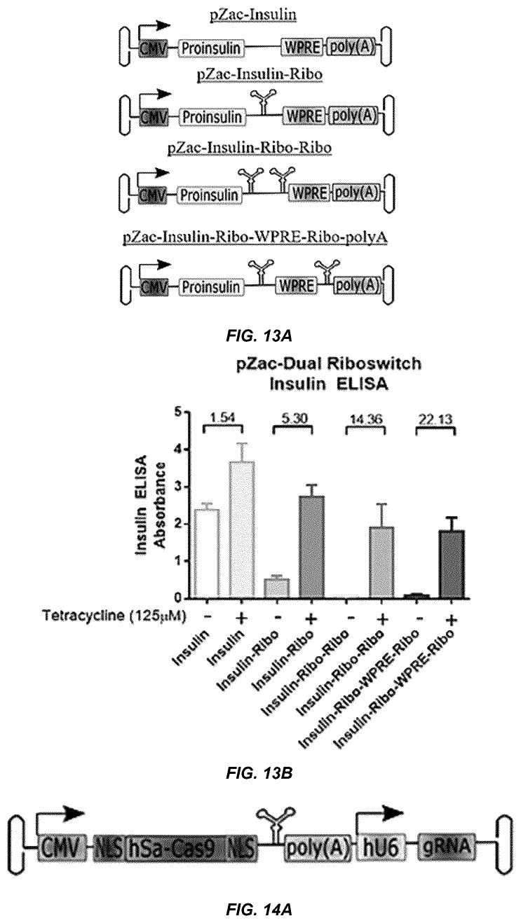

The disclosure exemplifies the tetracycline induced expression of insulin by cloning the tetracycline hammerhead aptazyme into the 3′ UTR of a modified insulin construct. The proinsulin sequence was modified with a H10D, K29R, R31K, and L62R mutations to enable the processing of proinsulin to mature insulin by a furin protease in organs outside of the pancreas. As shown in FIG. 12B, upon transfection into HEK293T cells in vitro, induction of insulin secretion into the cell culture supernatant was accomplished upon the addition of tetracycline (125 μM). This effect was shown to be dependent upon the presence of the tetracycline-hammerhead aptazyme as the positive control (AAV-Insulin) shows no tetracycline induced fold change in secreted insulin expression. As it is advantageous to control the expression of insulin on a patient-by-patient basis, the disclosure further demonstrates that the tetracycline-induced expression of mature insulin occurs in a tetracycline dose-dependent manner (FIG. 12C). To demonstrate the tunability of insulin expression dynamics with this ribozyme-based approach several versions of the insulin riboswitch designs was generated and cloned into the pZac AAV backbone. The number and location of the tetracycline-hammerhead aptazymes (FIG. 13A), and then quantified the amount of insulin that was secreted in the cell culture supernatant of HEK293T cells 48 hours after transfection upon addition of either DMEM or tetracyline (125 μM) 4-6 hours after transfection. As shown in FIG. 13B, the addition of multiple aptazymes resulted in lower background (no tetracycline) expression of insulin. Due to these low background levels, greater than 20-fold induction of secreted insulin with one of the constructs upon the addition of tetracycline.

Through this 3′UTR aptazyme based approach, the disclosure demonstrates the feasibility of utilizing this approach to address the insulin needs of diabetic patients. The disclosure shows in vitro that the levels of mature insulin expression are dose-dependent and tunable with engineering of the construct.

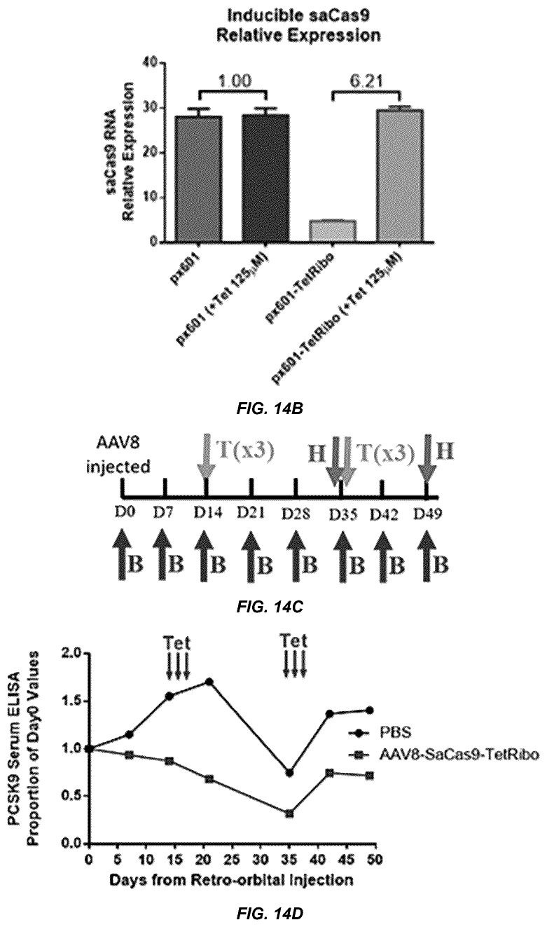

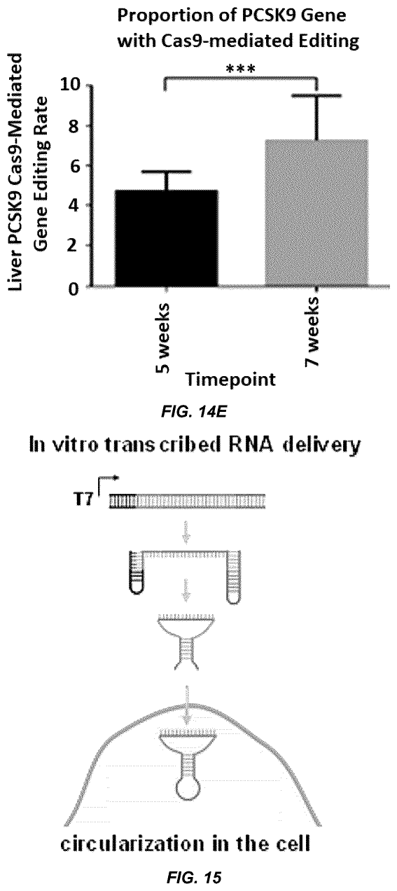

The feasibility of this disclosure is also shows by first demonstrating its effectiveness with a 3′UTR tetracycline-hammerhead aptazyme, as shown in FIG. 12A. The px601 plasmid backbone was used and the aptazyme riboswitch cloned into the 3′ UTR between the SaCas9 protein and the bGH poly(A) sequence (FIG. 14A). The expression of SaCas9 was demonstrated to be tetracycline-inducible in vitro by transfecting HEK293T cells with the plasmid and changing media 4-6 hours after transfection to either DMEM alone or DMEM containing 125 μM tetracycline. RNA was isolated 48 hours after transfection, reverse transcribed, and the relative expression of the SaCas9 transcript was quantified relative to GAPDH through a qPCR assay. As shown in FIG. 14B, a reduction in the background was observed, no ligand condition with an increase in expression upon the addition of tetracycline-containing media (˜6-fold).

The disclosure demonstrates tetracycline-inducible genome editing by SaCas9 in vivo using AAV8 capsid with the construct of FIG. 14B. For these in vivo experiments a SaCas9-specific single-guide RNA targeting the mouse Pcsk9 gene was cloned in to the vector. These capsids were then administered to 6-8-week-old C57Bl/6 mice via retro-orbital injection at a dose of 5E12 viral genomes/mouse. Serum was collected every week and administered tetracycline (30 mg/kg) at the 2- and 5-week timepoint was performed. Additionally, at the 5- and 7-week timepoints, the livers from a subset of the mice were harvested, DNA was isolated, and the Cas9-induced Pcsk9 genome editing was quantified using Synthego's Inference for CRISPR Edits (ICE) tool (FIG. 14C). As shown in FIG. 14D, a decrease was observed in serum PCSK9 protein levels, as measured by a mouse PCSK9 ELISA kit, following the first 3-day tetracycline-administration, suggesting that the gene editing induced by Cas9-mediated gene knockdown was ligand-dependent. When the livers were harvested and the proportion of DNA edited at the Pcsk9 locus was quantified, an effect of DNA editing was observed based on the tetracycline regimen received by the mouse groups. At the 5-week timepoint following one round of a three-day tetracycline regimen, <5% editing of the Pcsk9 locus was quantitated, while an additional three-day tetracycline regimen increased the proportion of edited DNA to around 78 (FIG. 14E). Through this experiment, the level of DNA editing mediated by SaCas9 is shown to be tunable based on the tetracycline regimen when utilizing the AAV-Cas9-Riboswitch based approach.

Persistence of RNA constructs. An RNA construct was generated by transcribing a DNA nucleic acid having a T7 promoter operably linked to a DNA construct encoding a 5′ P3 twister ribozyme, a 5′ ligation sequence, an IRES operably linked to a coding sequence for GFP, a linker and 3′ ligation sequence and a 3′ Twister ribozyme followed by a poly-T tail to obtain a linear RNA construct (see FIG. 16A).

293T were seeded at 25% confluency in 12 wells and transfected with lipid-RNA complexes consisting of 1 μg of mutated circular or circular GFP RNA and 3.5 μL Lipofectamine MessengerMax. RNA was isolated from cells over three days using the Qiagen RNeasy Kit and qPCR was performed to determine the amount of circularized RNA. **p<0.01 t-test comparison within each day. (See FIG. 16B-D).

293T were seeded at 25% confluency in 12 wells and transfected with lipid-RNA complexes consisting of 1 μg of mutated circular or circular GFP RNA and 3.5 μL Lipofectamine MessengerMax. RNA was isolated from cells over three days using the Qiagen RNeasy Kit and qPCR was performed to determine the amount of GFP RNA. *p<0.05 t-test comparison within each day.

293T were seeded at 25% confluency in 12 wells and transfected with lipid-RNA complexes consisting of 1 μg of mutated circular or circular zinc finger RNA and 3.5 μL Lipofectamine MessengerMax. DNA was isolated using the Qiagen DNeasy Blood & Tissue Kit, the edited region was amplified and Sanger sequenced, and editing efficiency was quantified using the Synthego ICE CRISPR Analysis Tool. 293T were seeded at 25% confluency in 12 wells and transfect with lipid-RNA complexes consisting of 1 μg Cas wildtype or variant RNA, 1 μg T2 guide RNA, and 3.5 μL Lipofectamine MessengerMAX. After 3 days, genomic DNA was isolated using the Qiagen DNeasy Blood & Tissue Kit, the edited region was amplified and Sanger sequenced, and editing efficiency was quantified using the Synthego ICE CRISPR Analysis Tool. (See FIG. 17A-C).

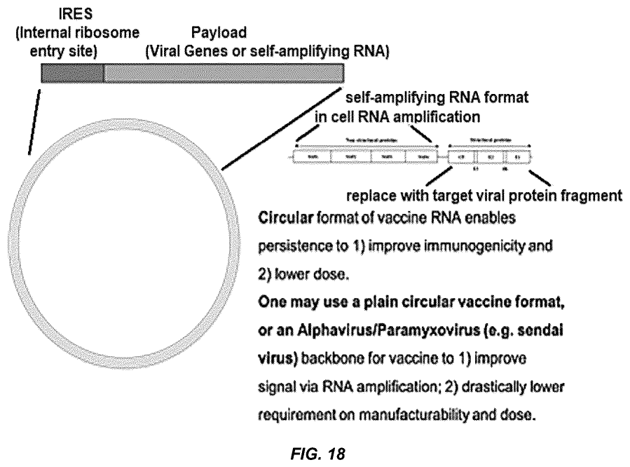

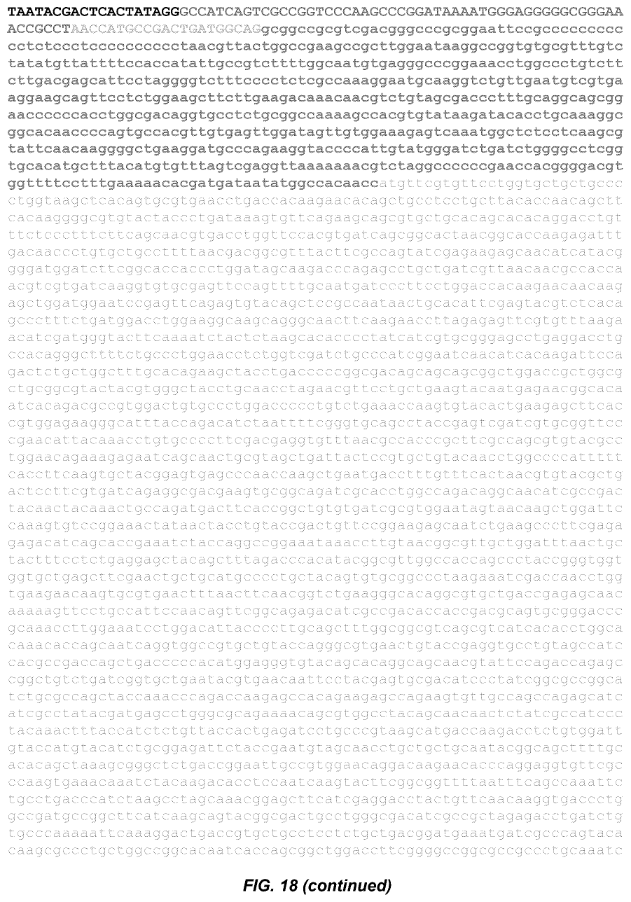

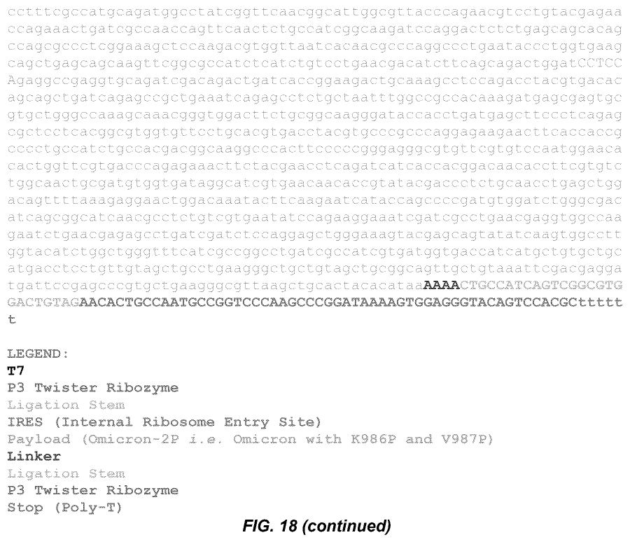

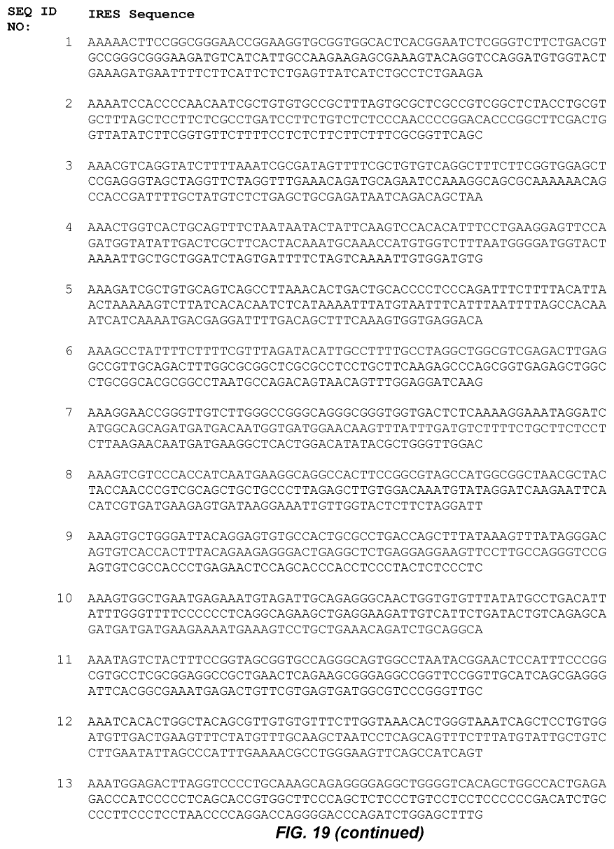

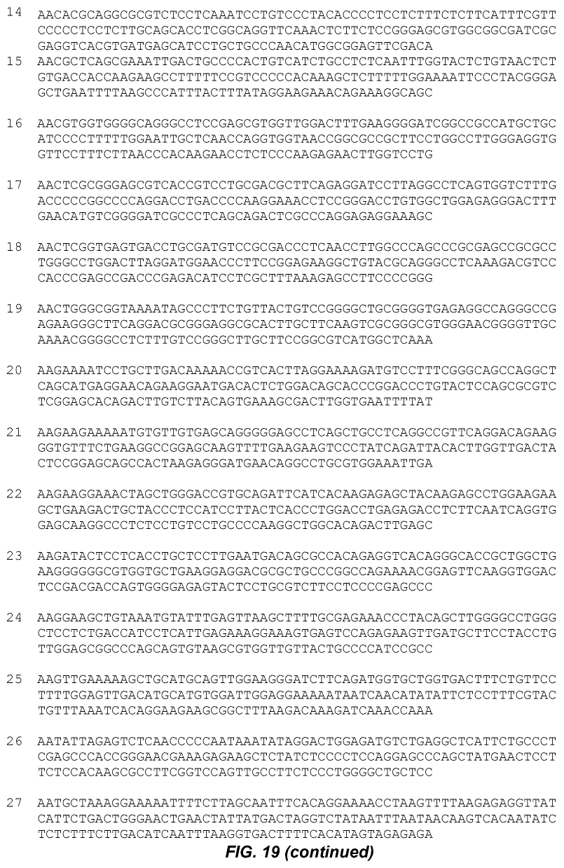

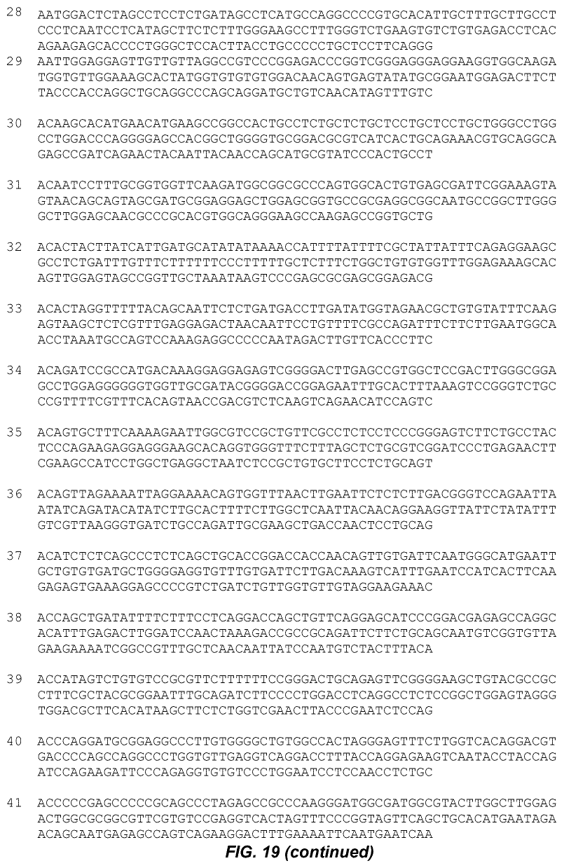

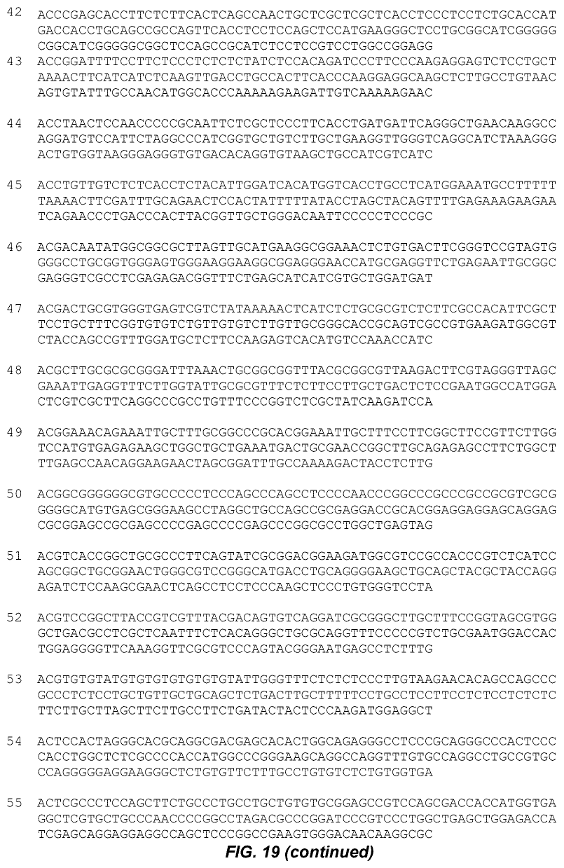

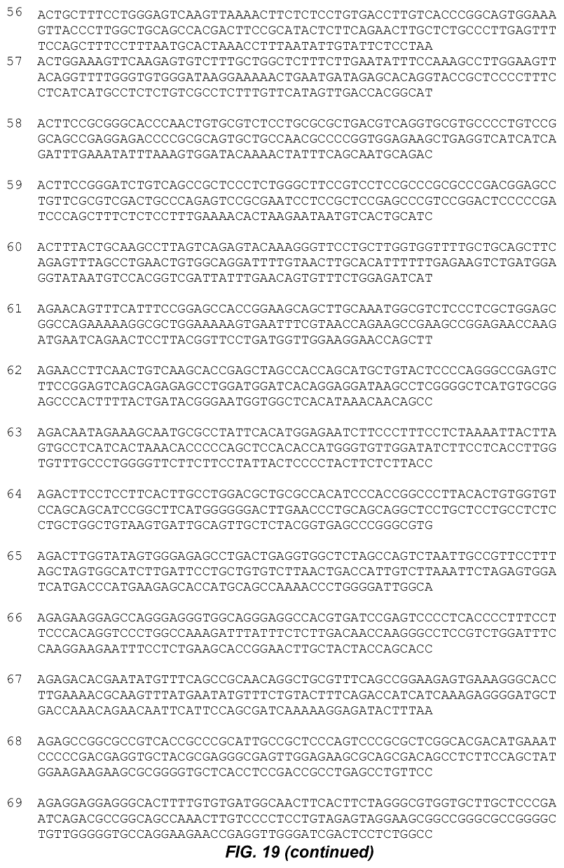

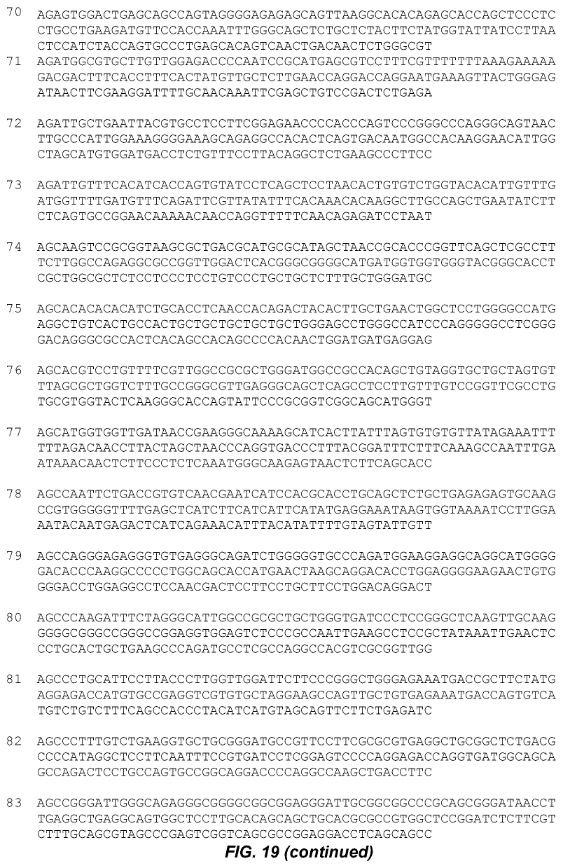

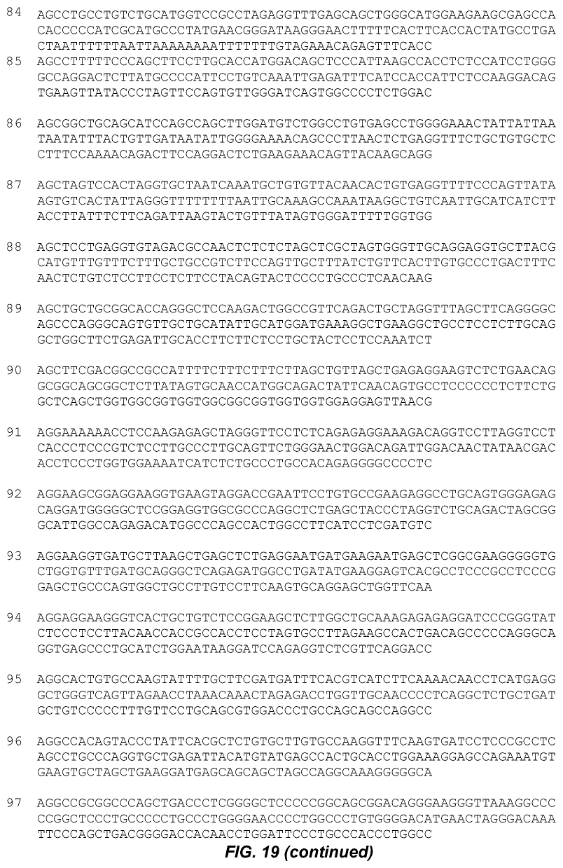

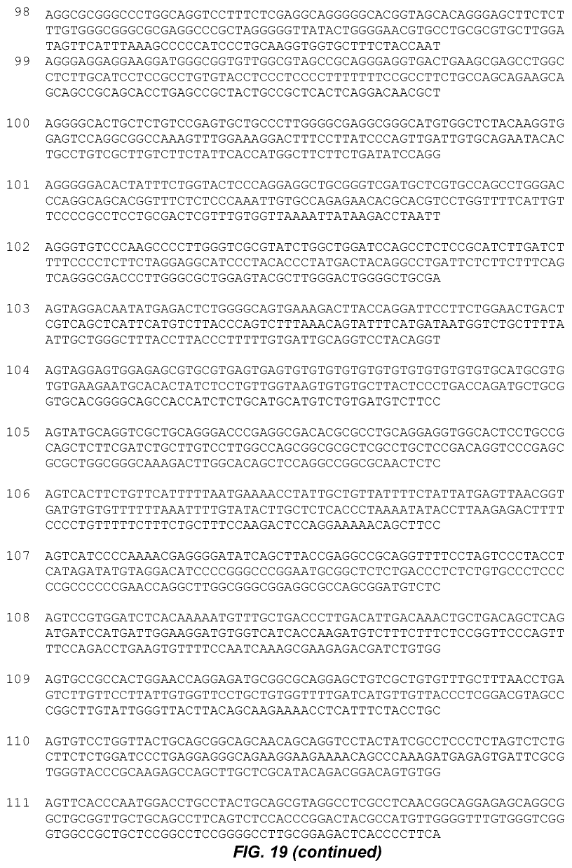

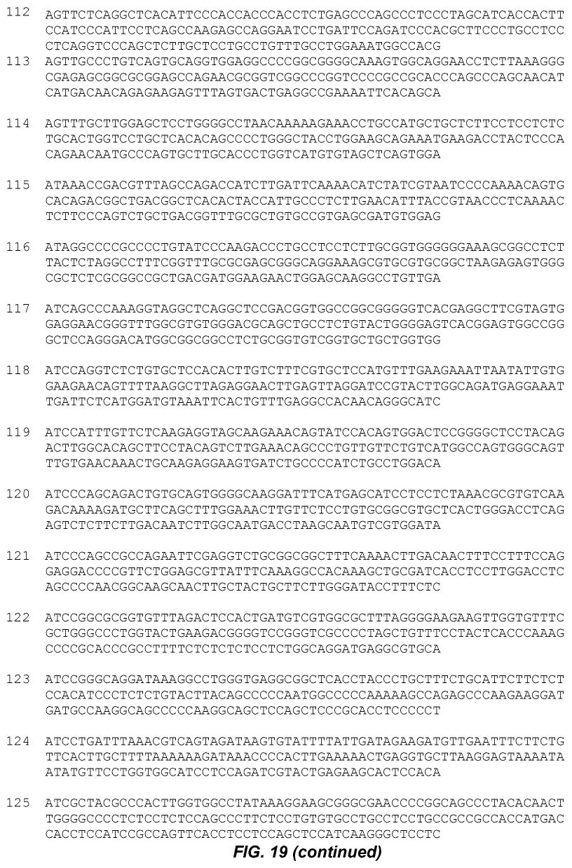

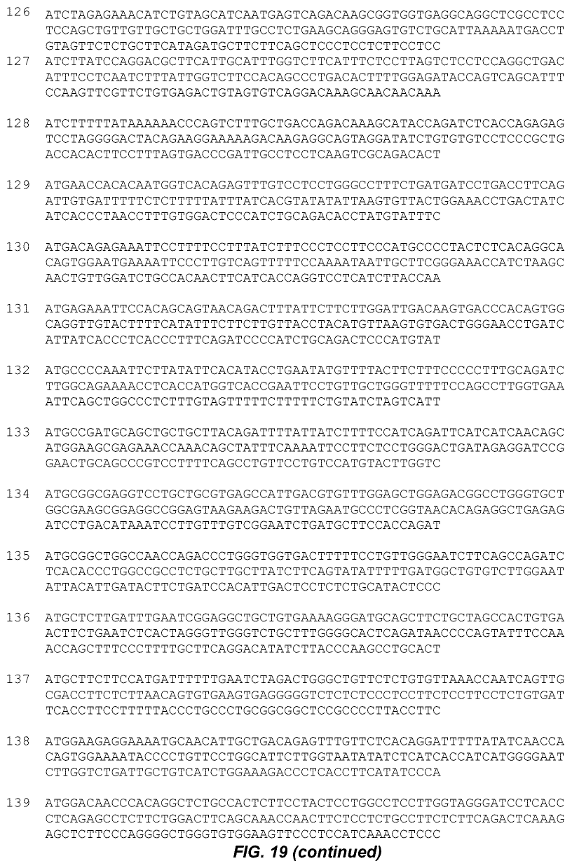

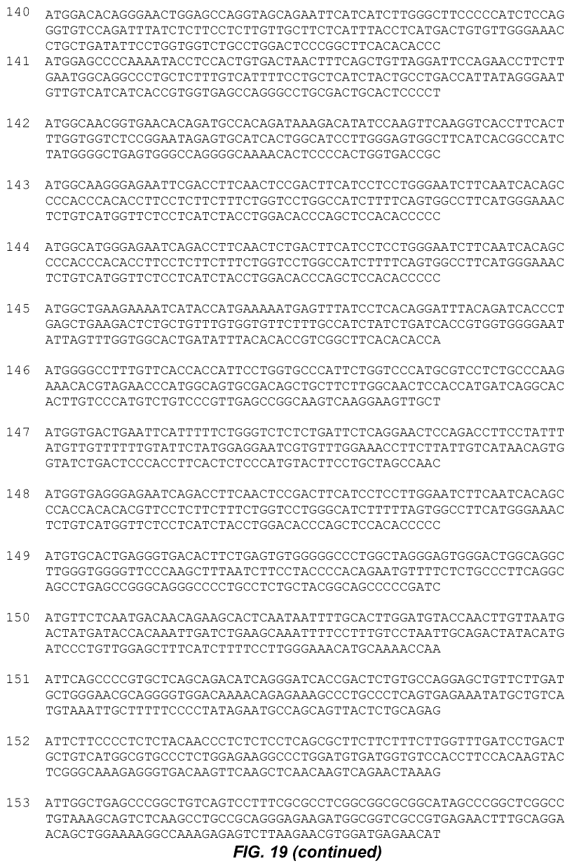

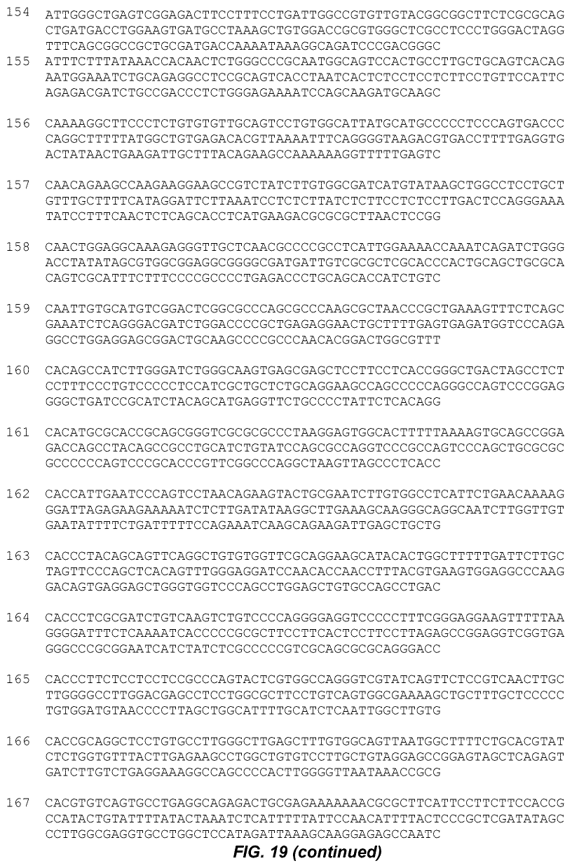

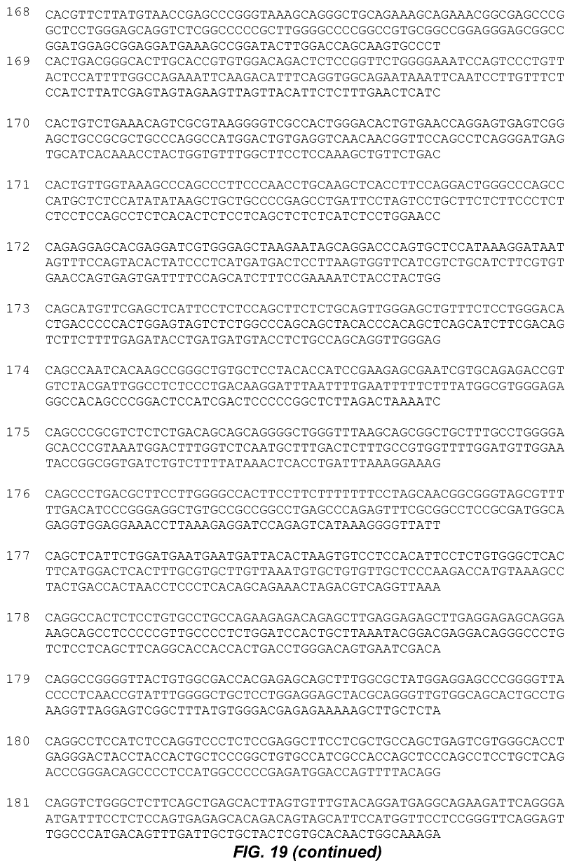

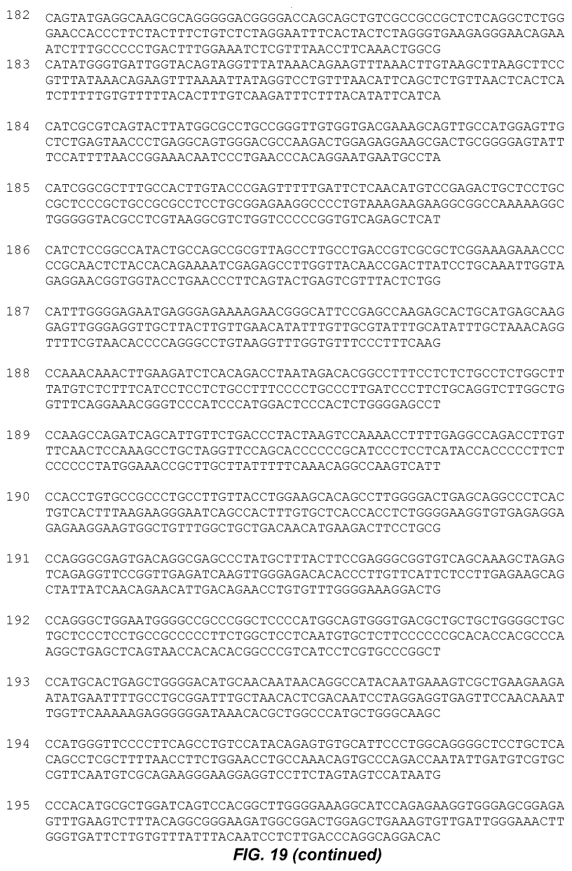

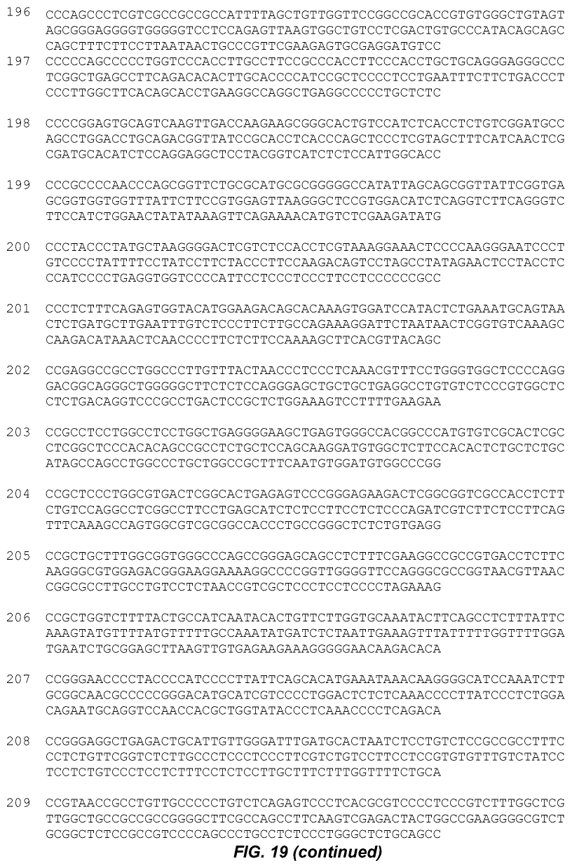

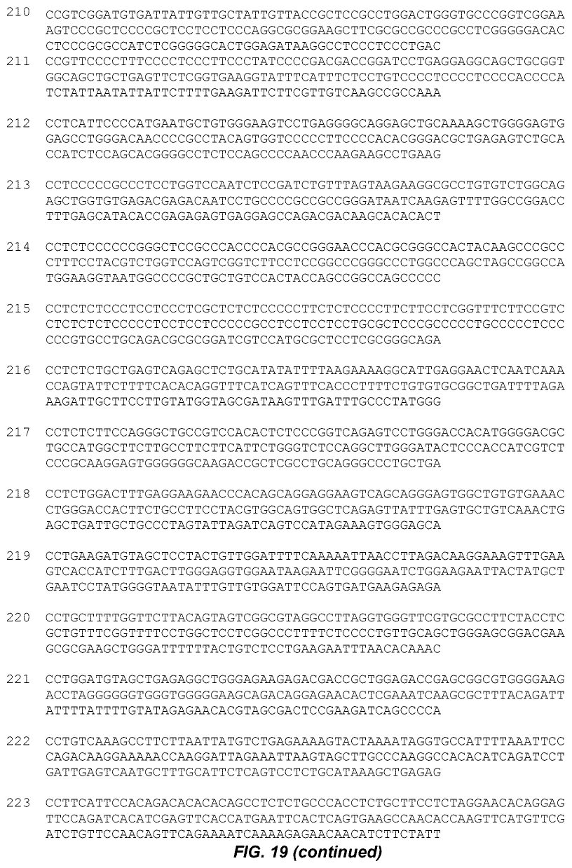

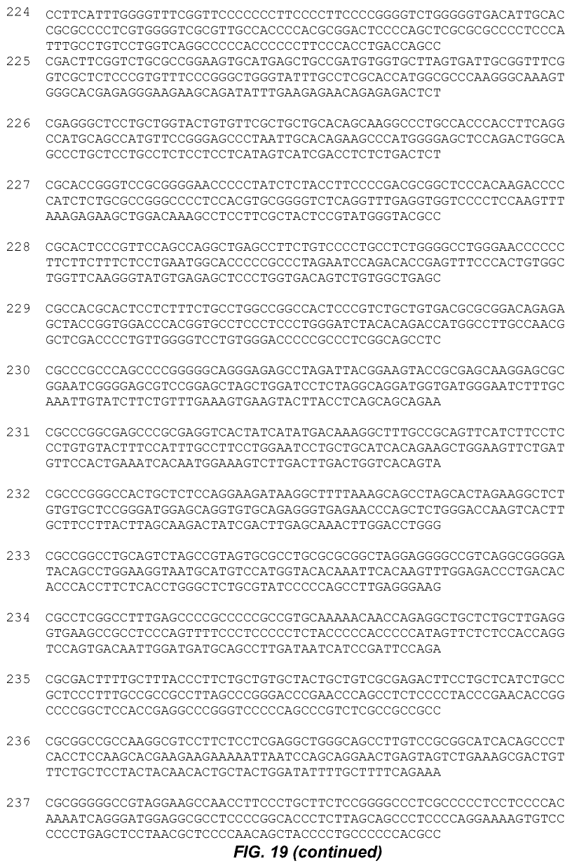

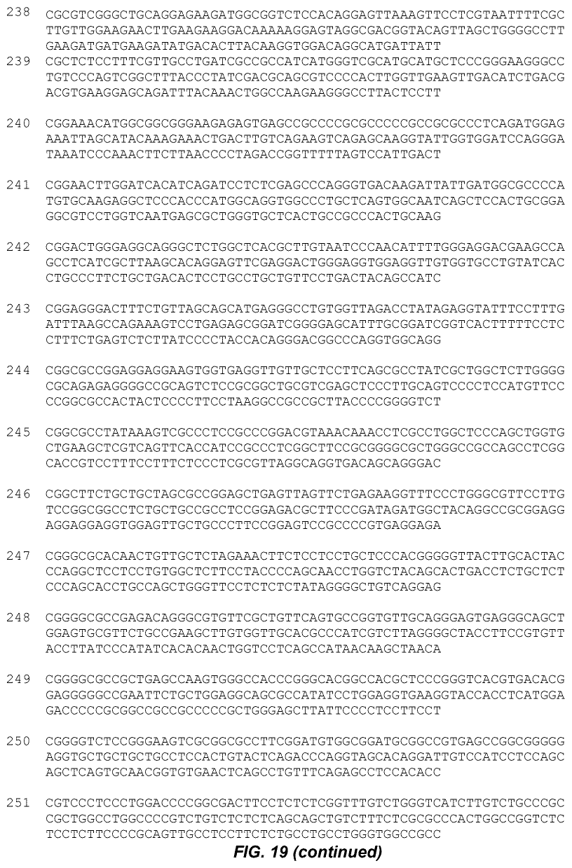

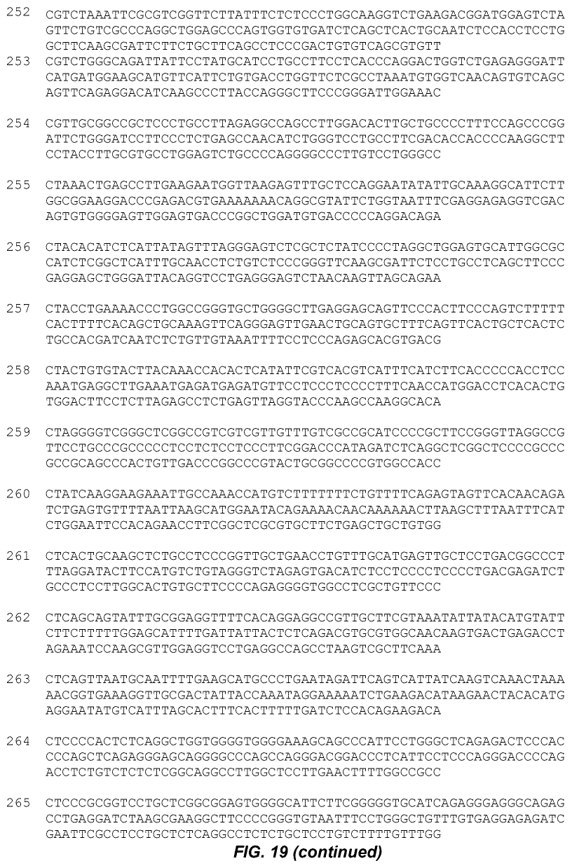

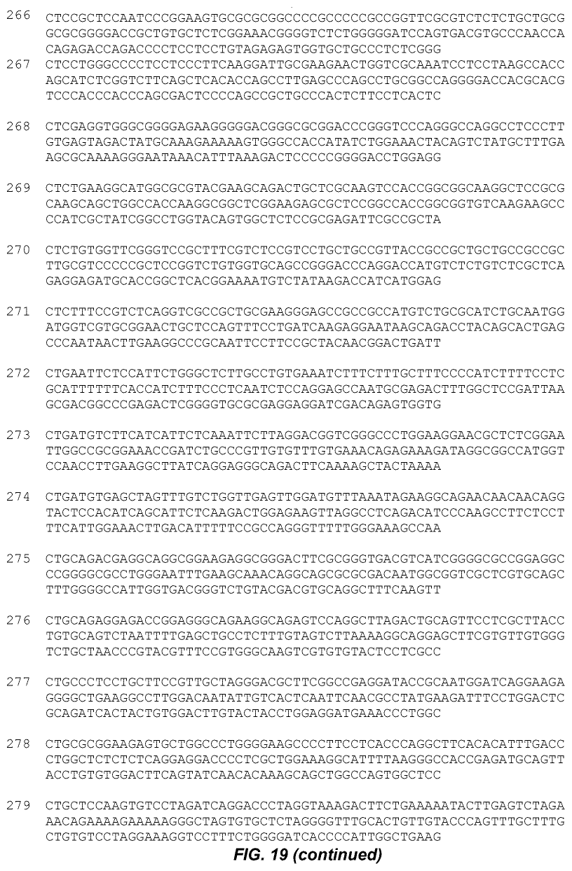

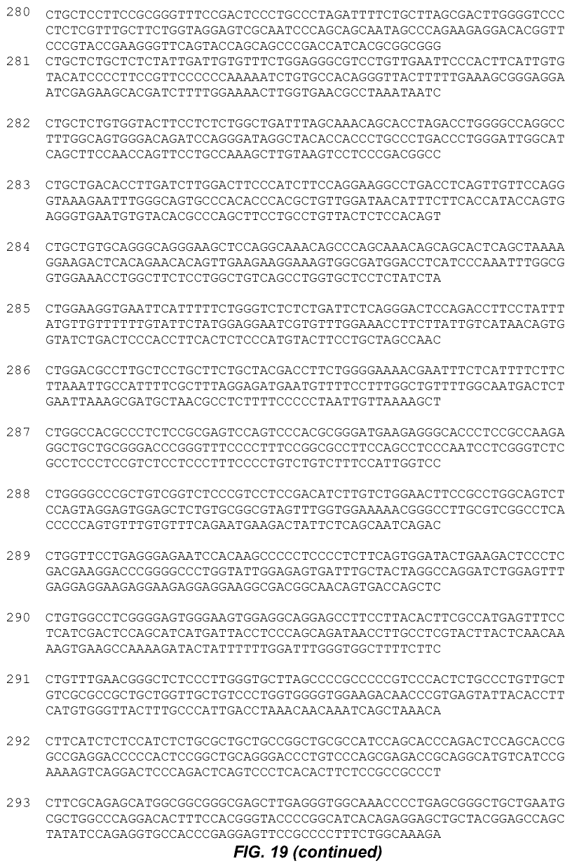

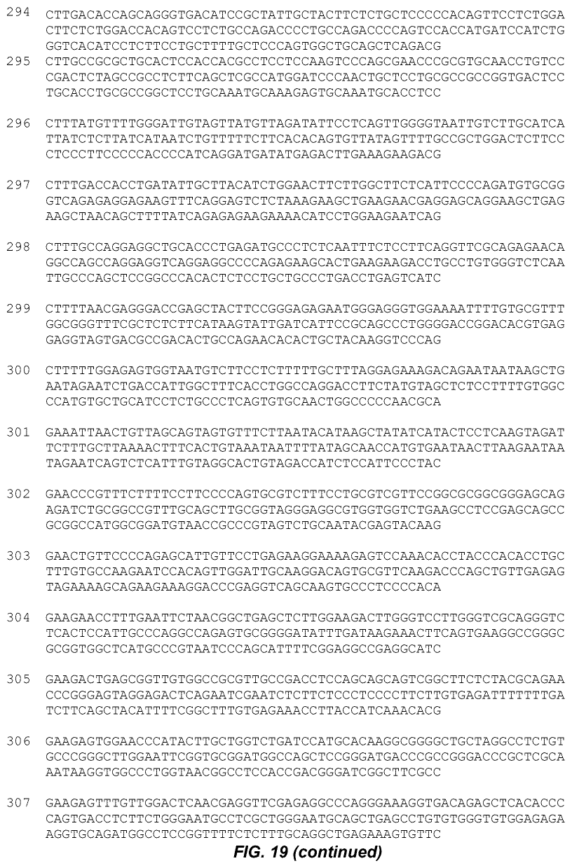

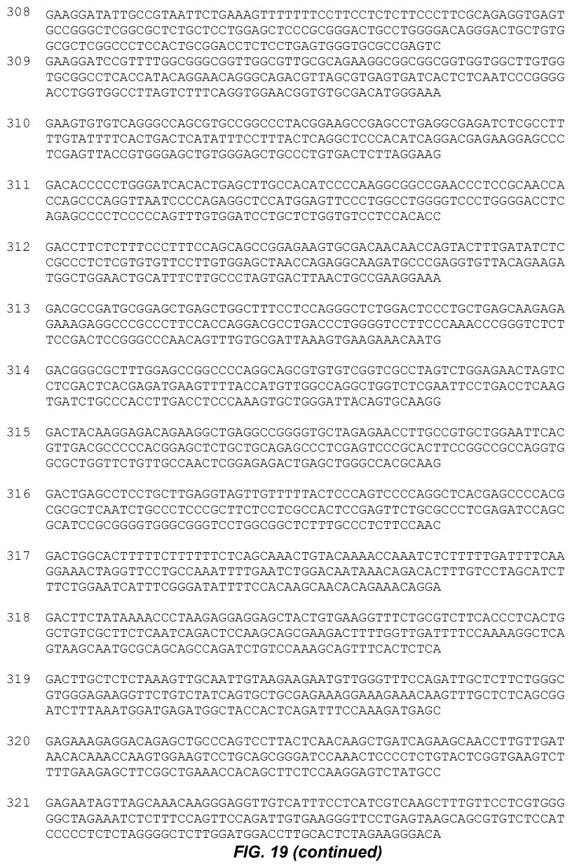

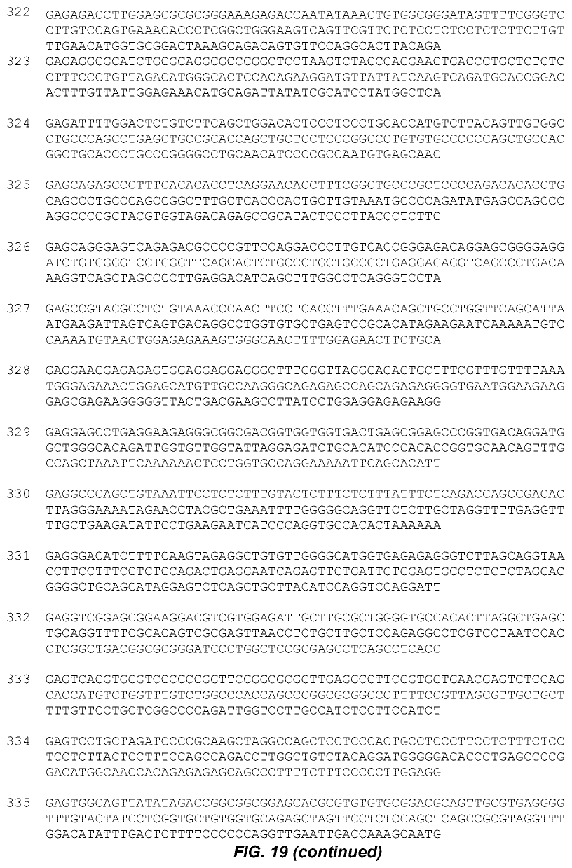

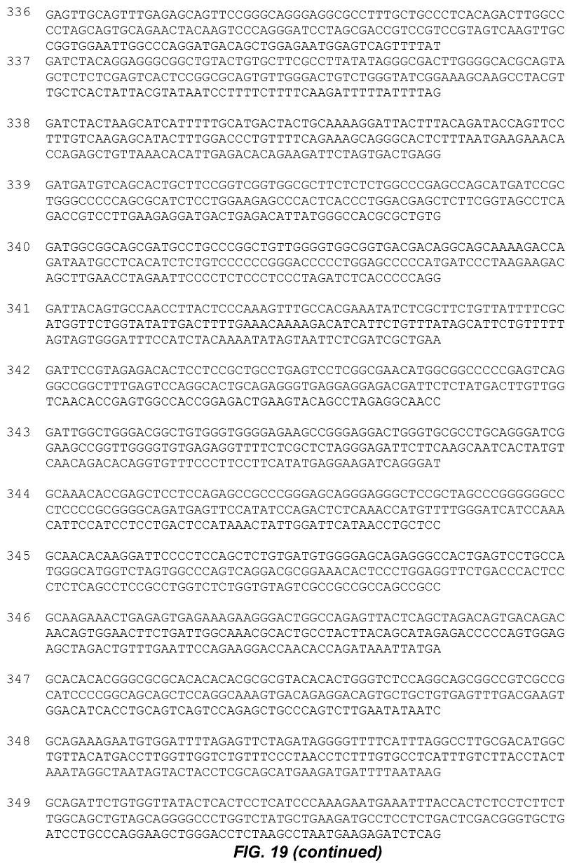

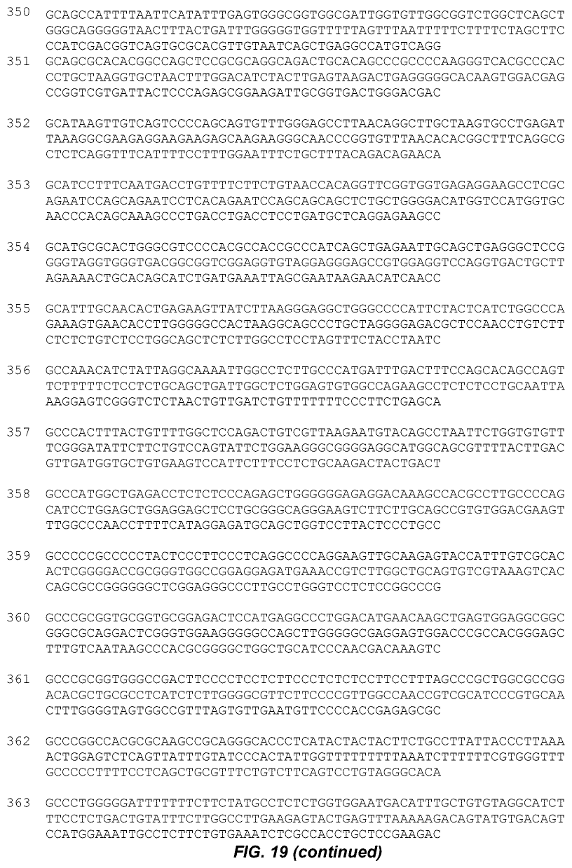

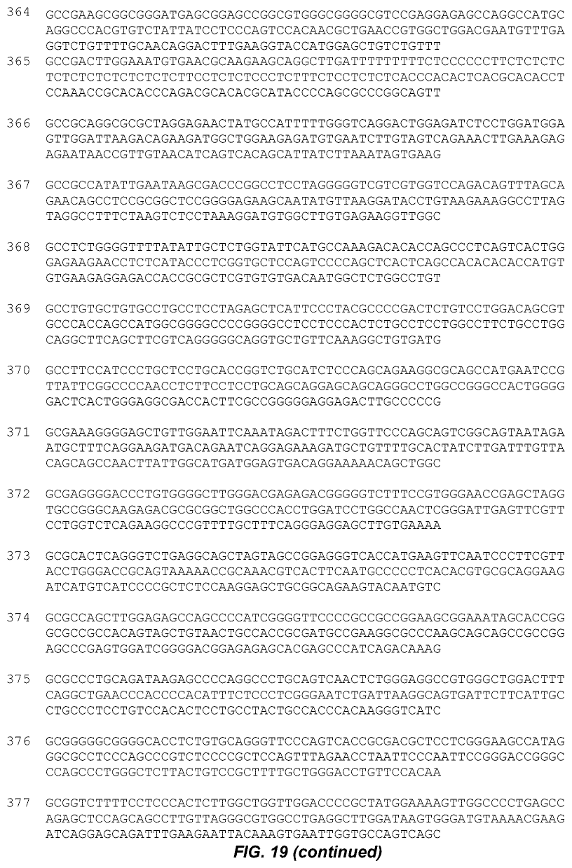

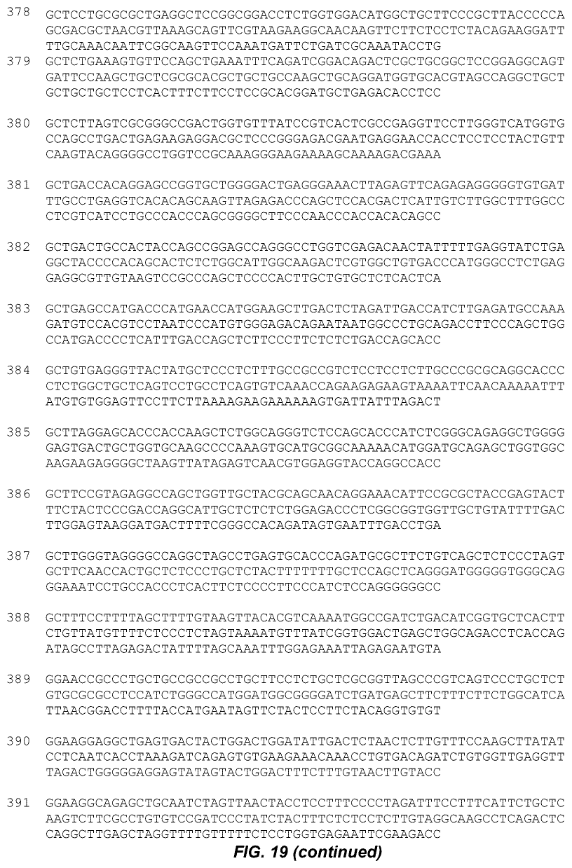

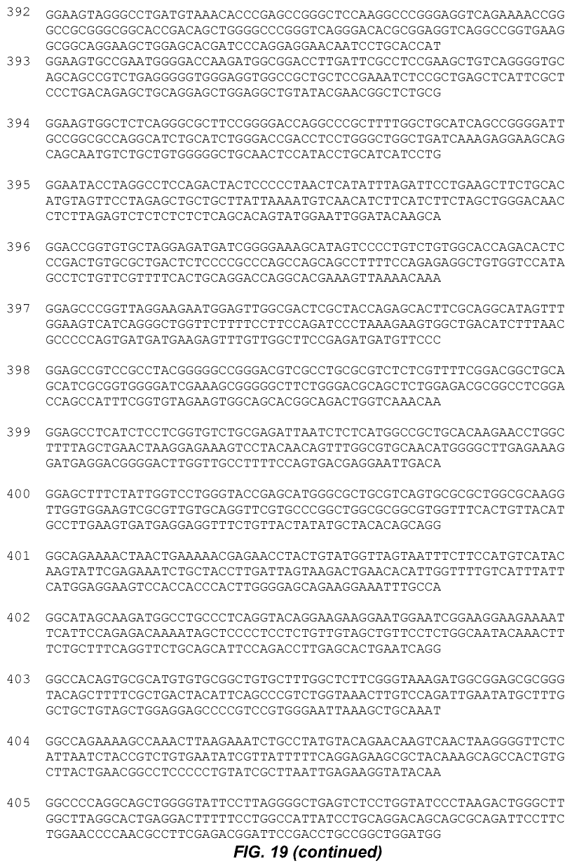

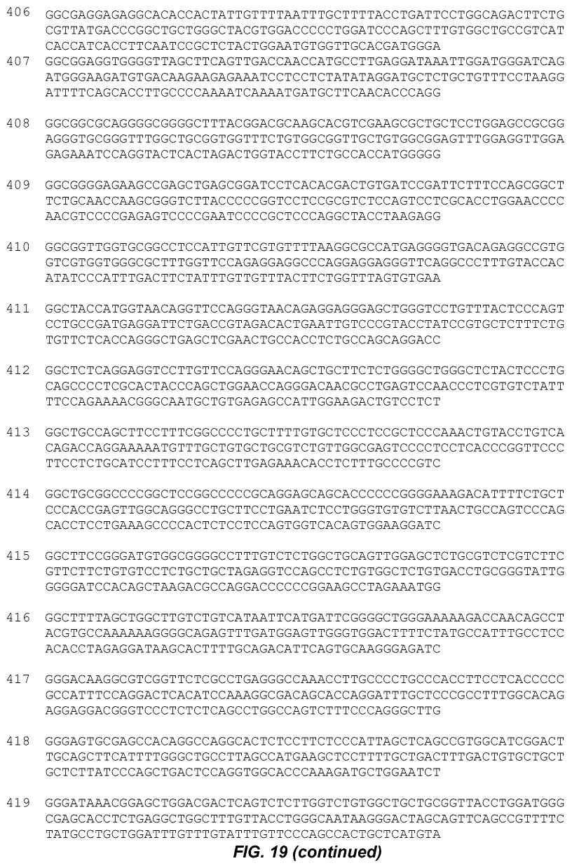

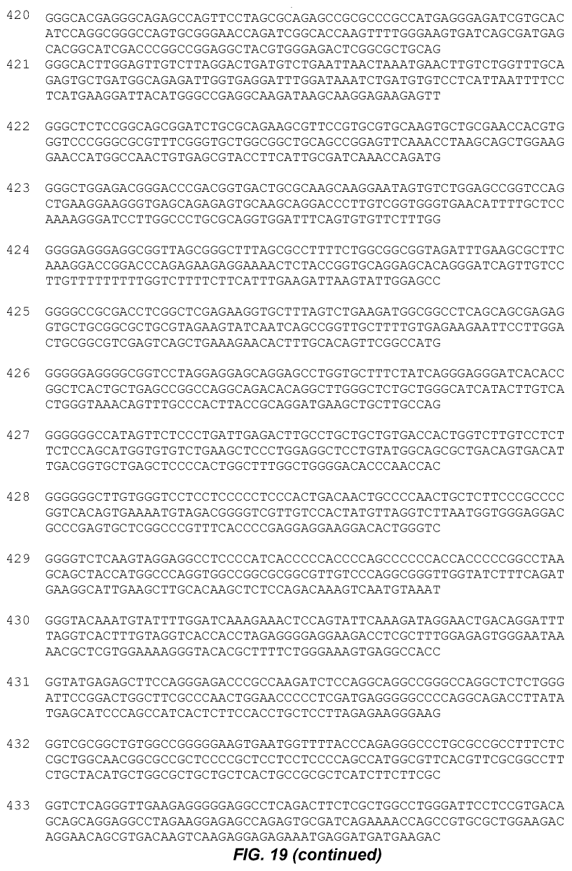

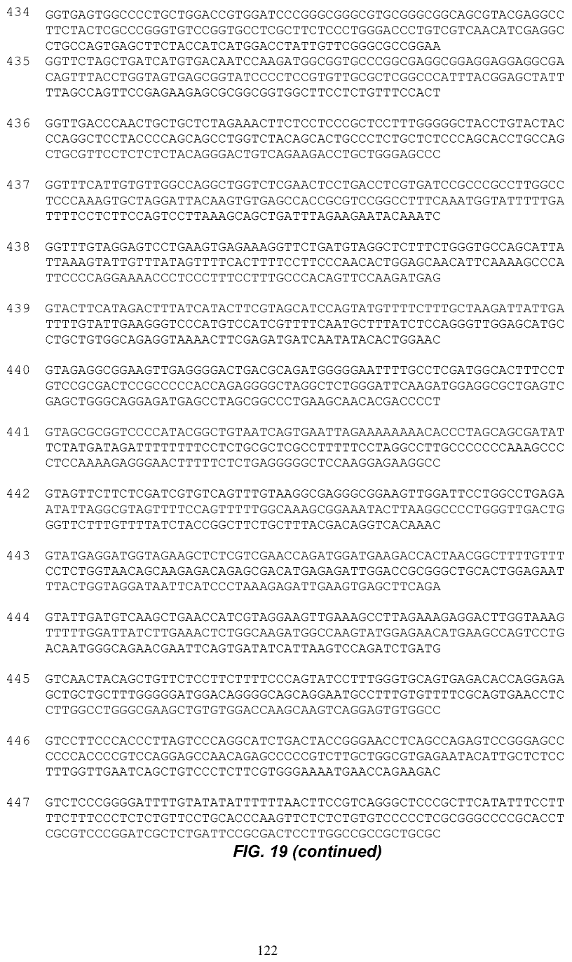

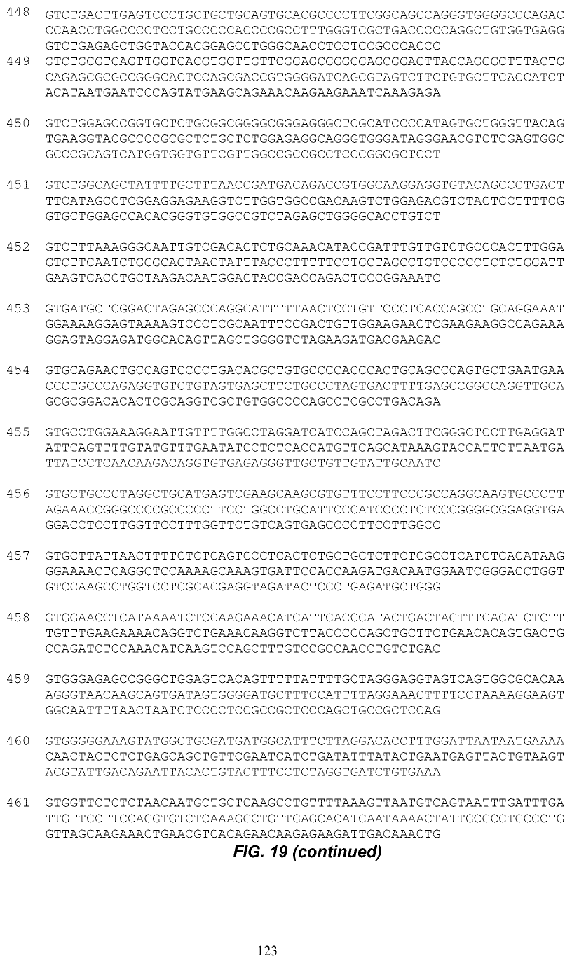

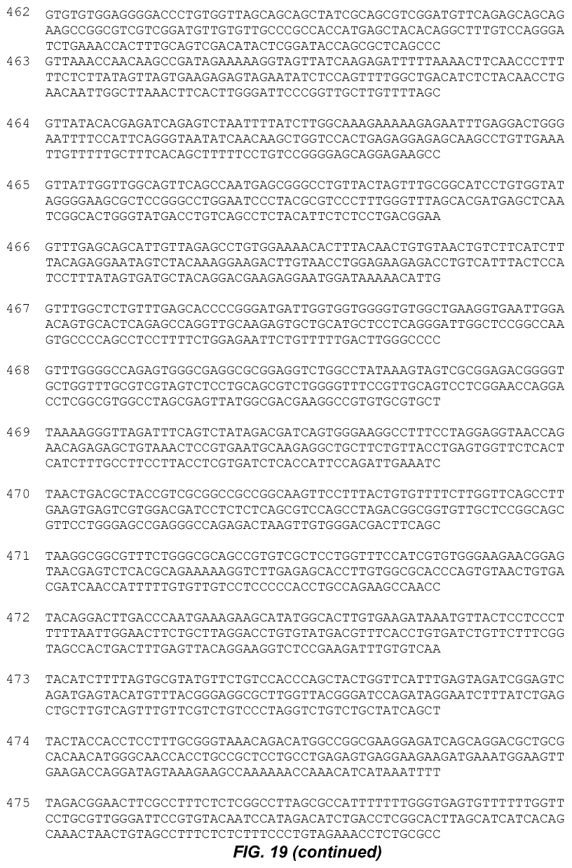

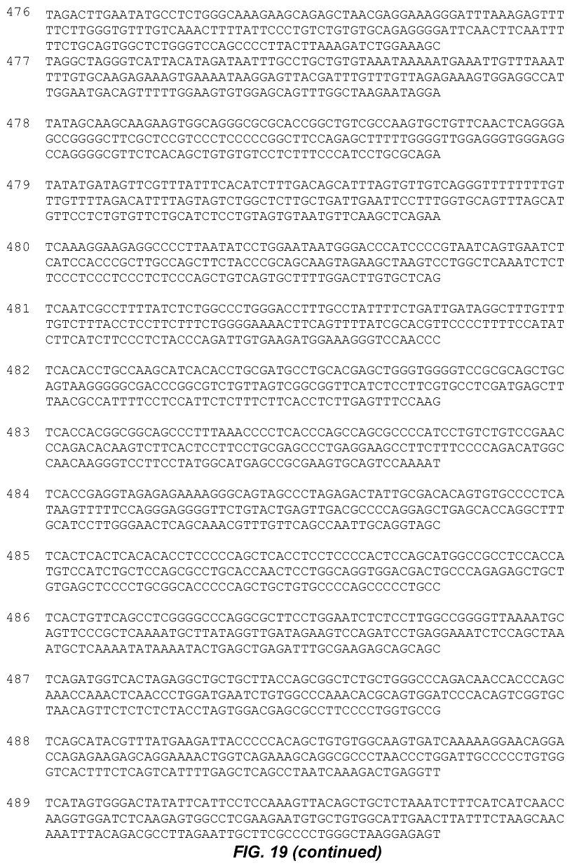

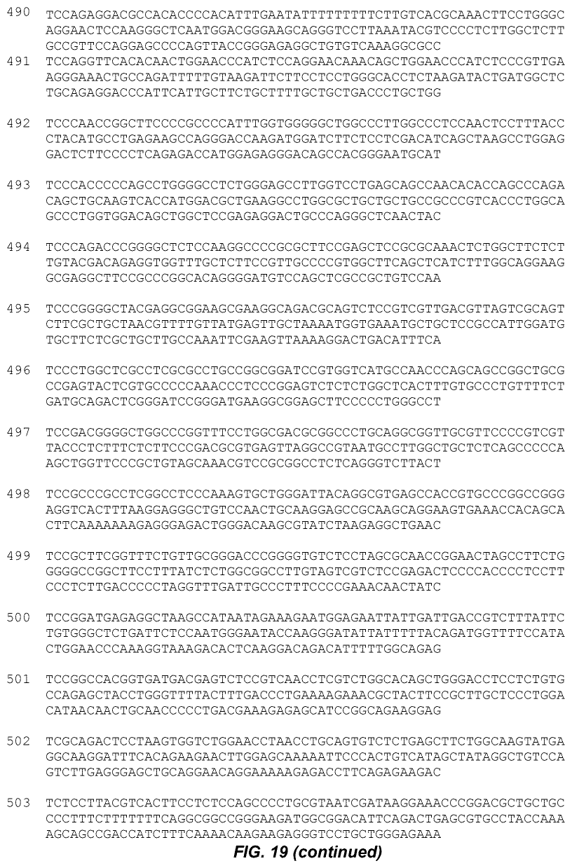

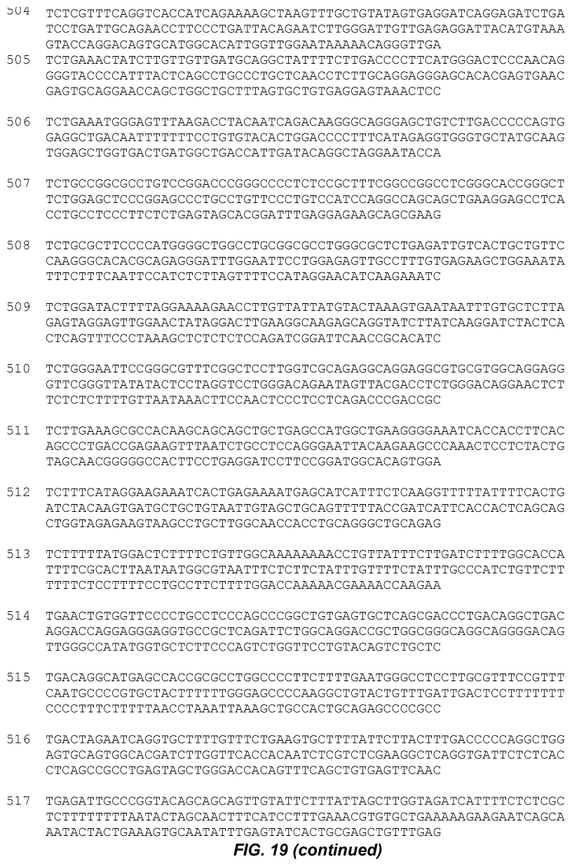

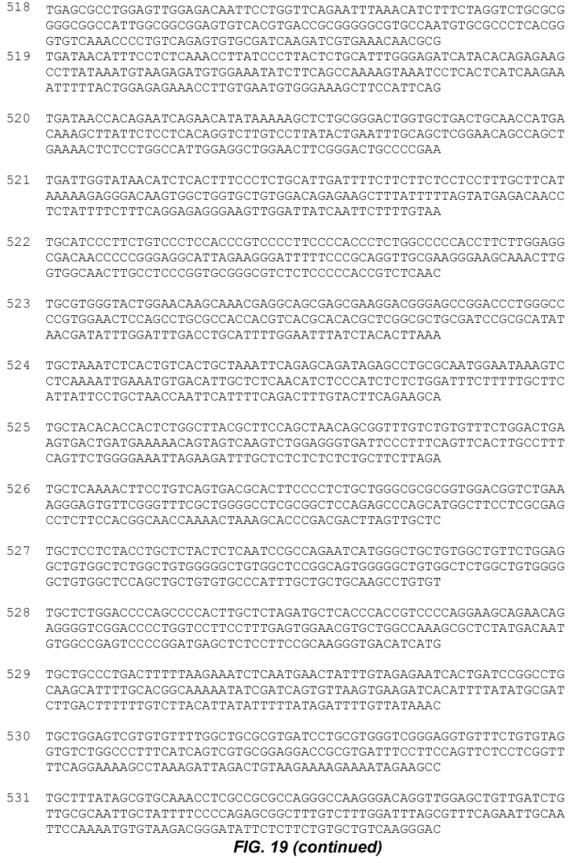

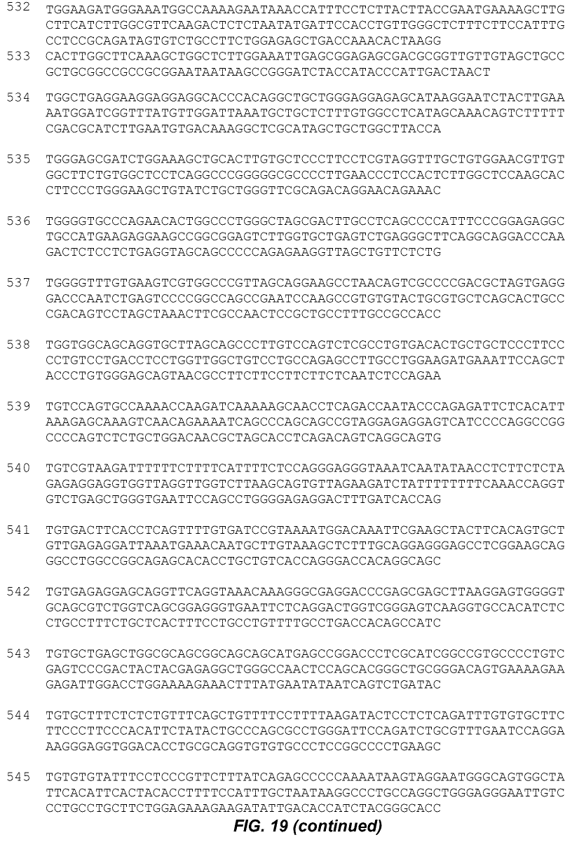

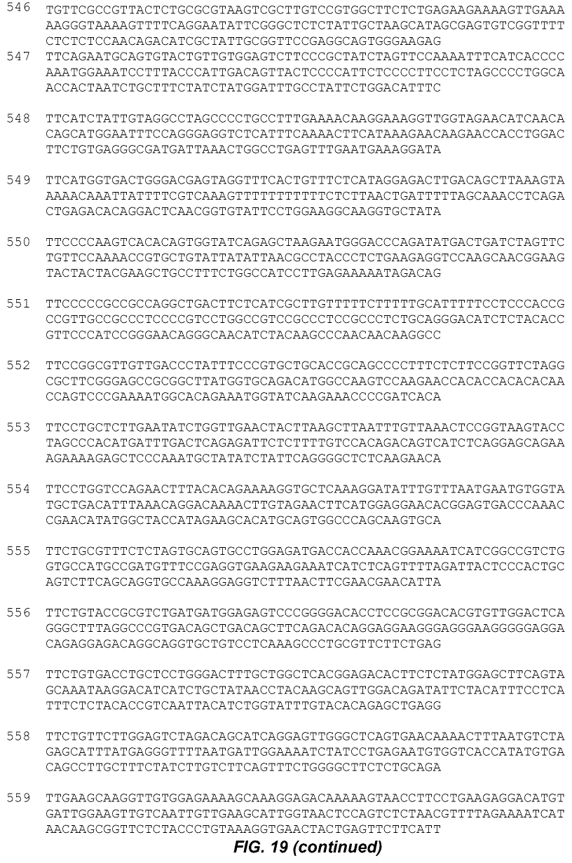

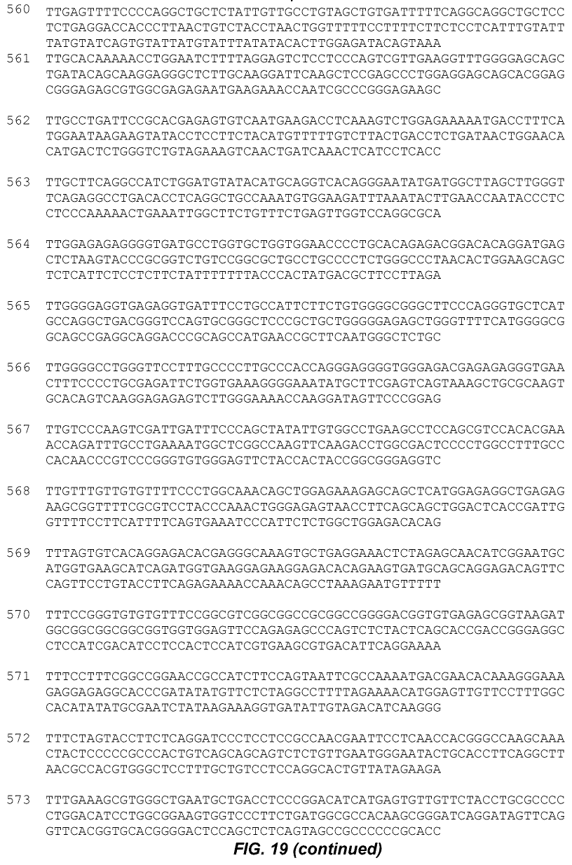

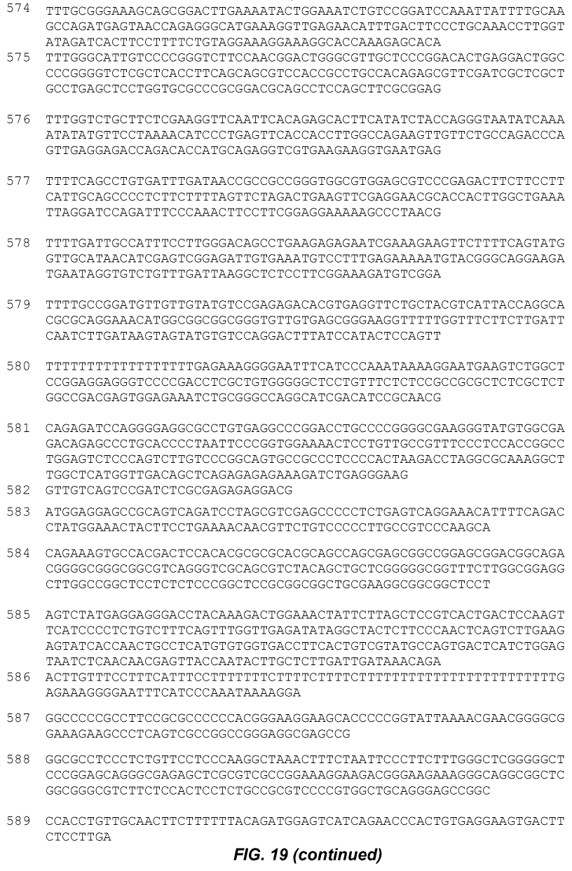

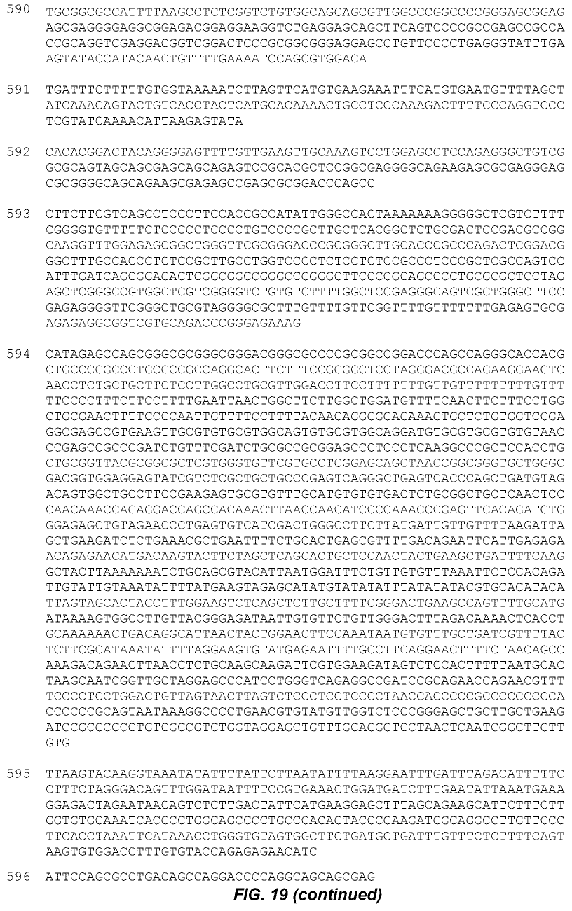

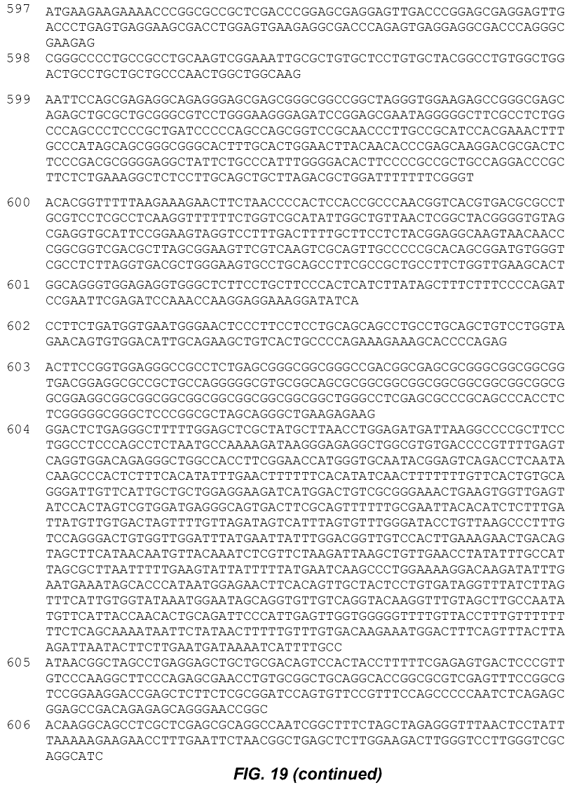

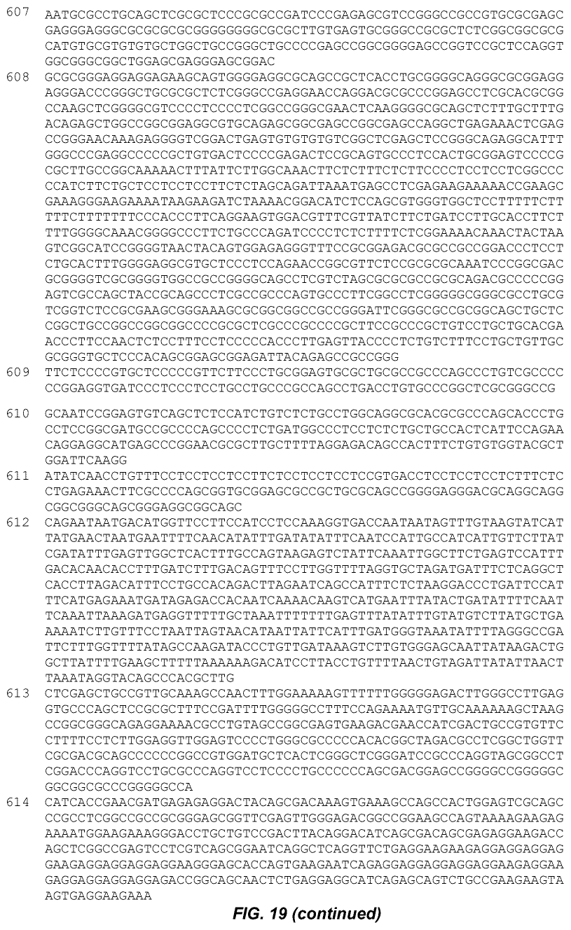

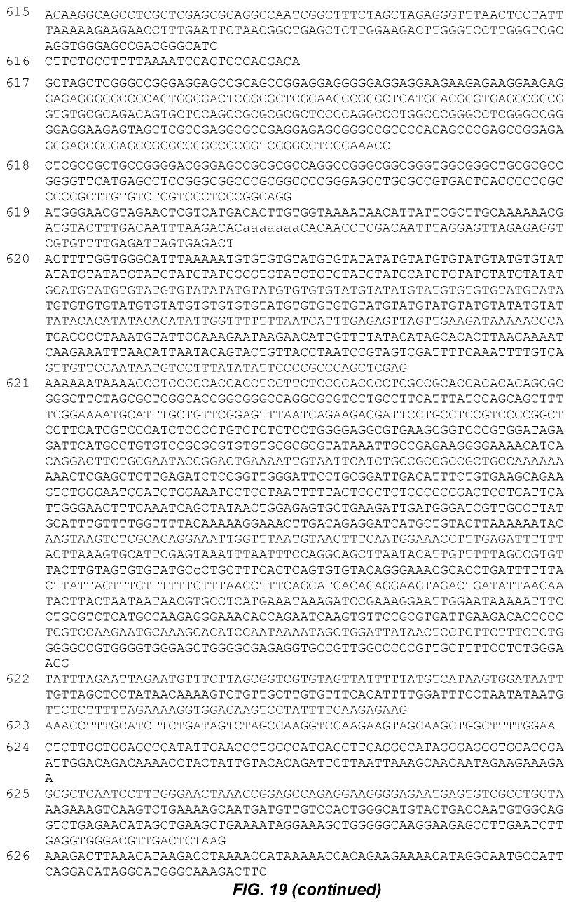

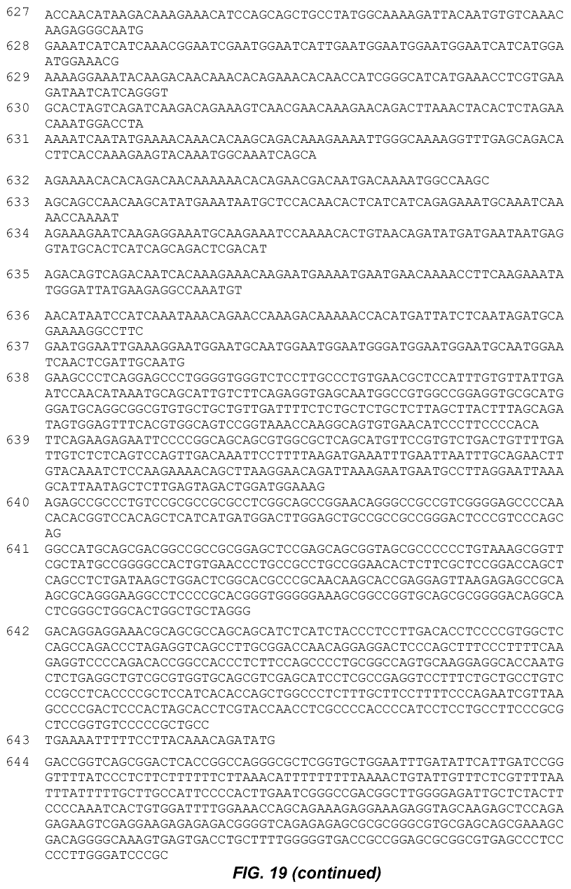

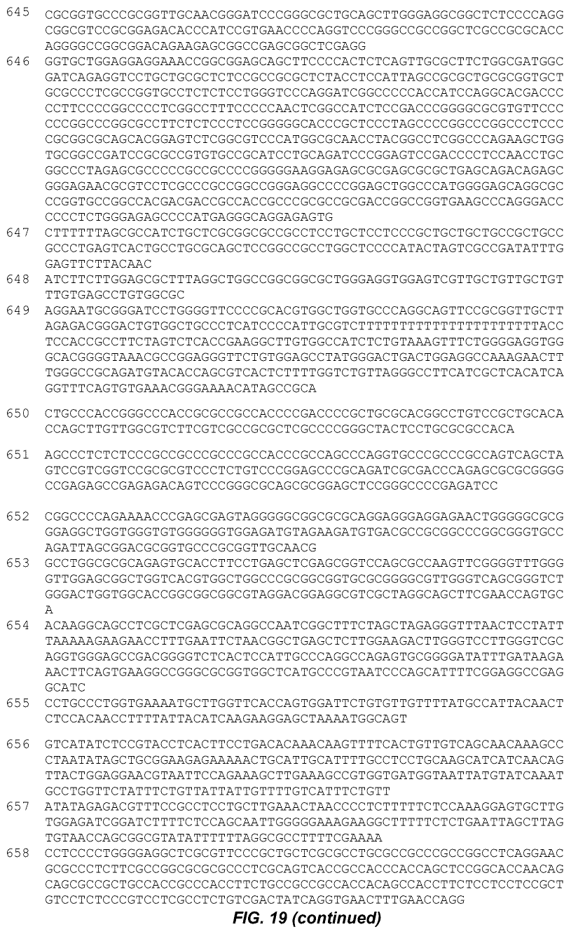

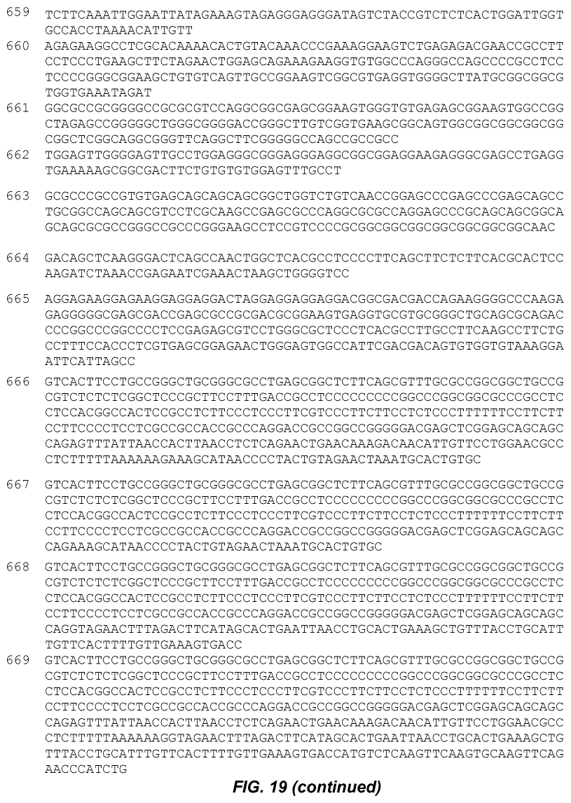

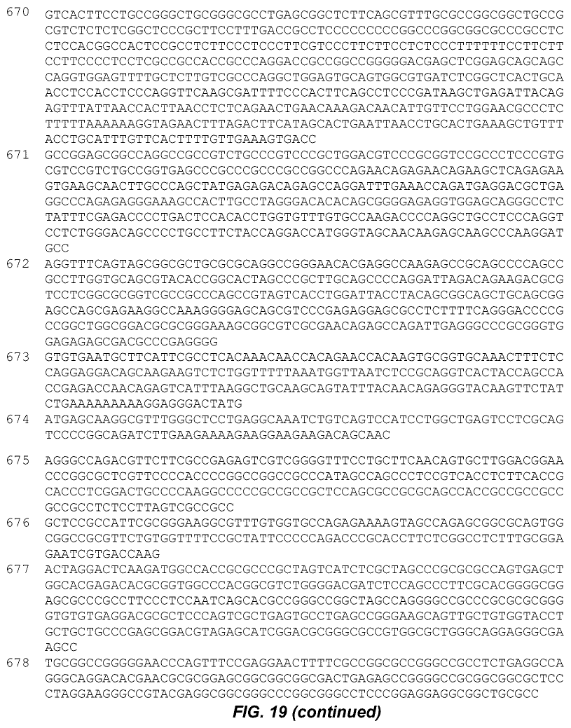

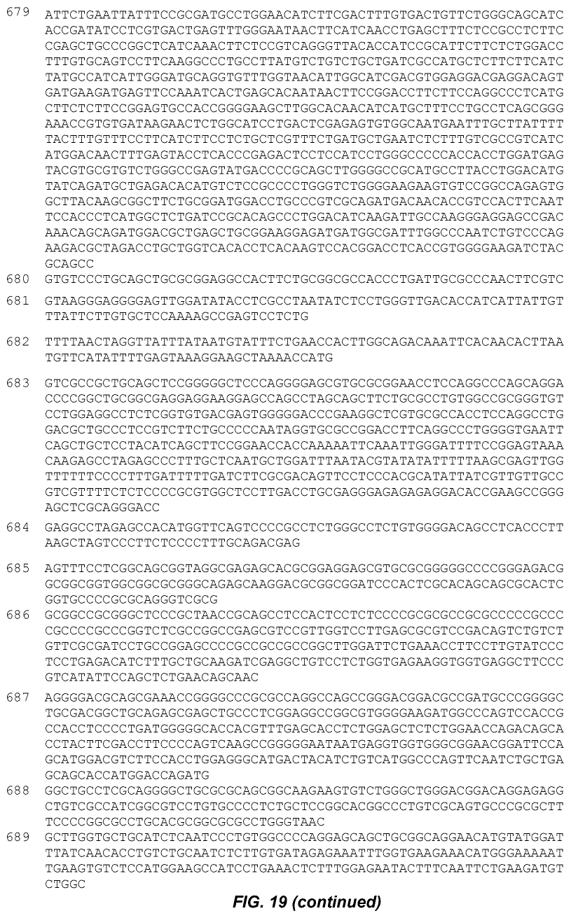

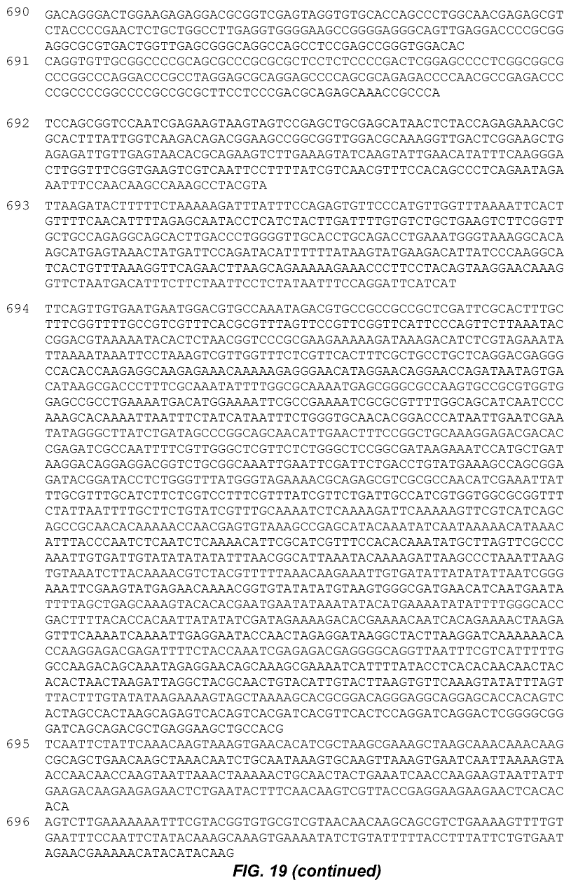

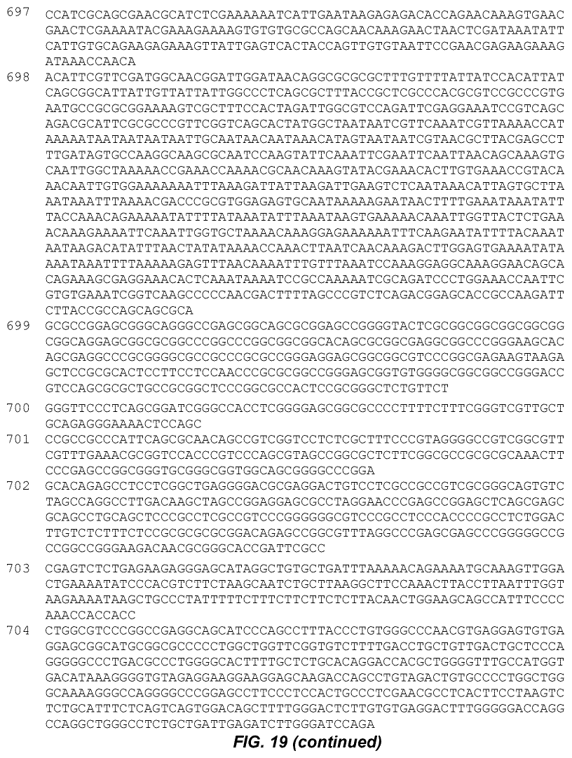

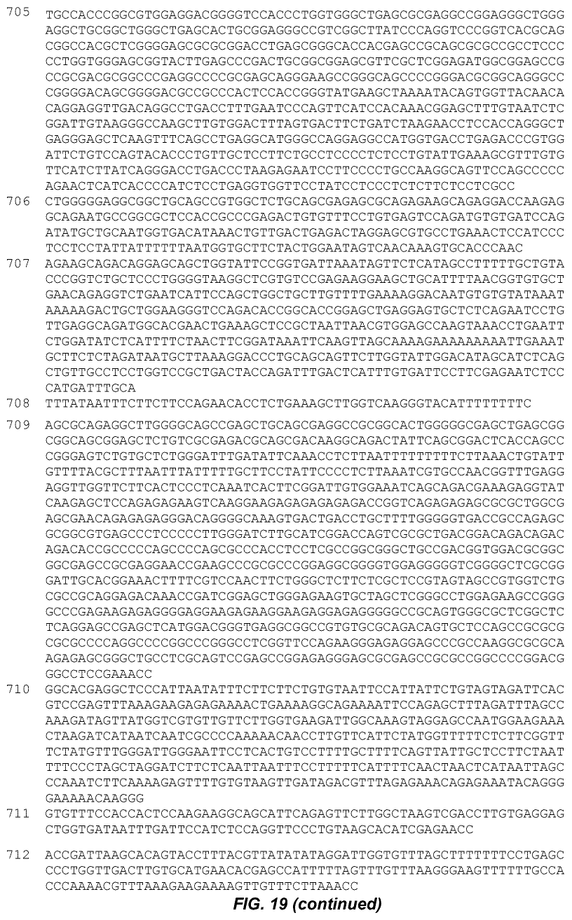

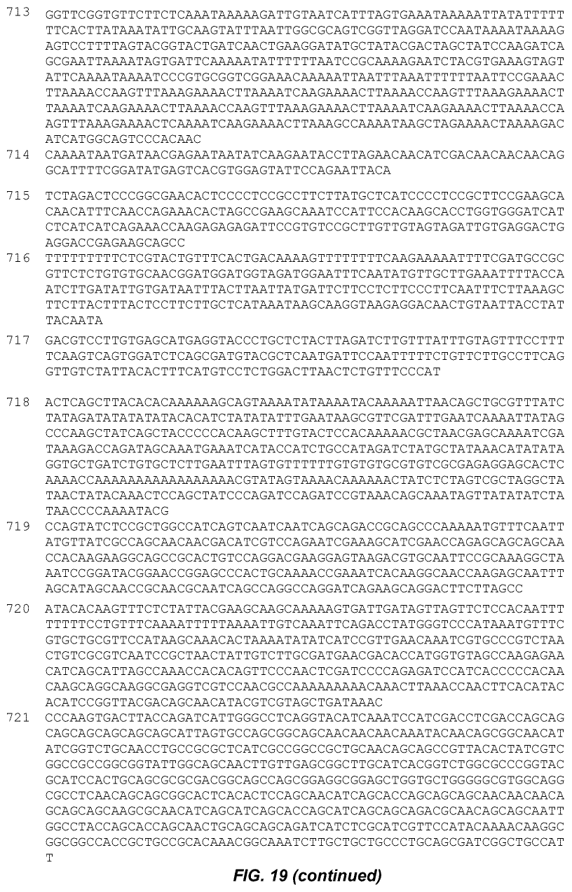

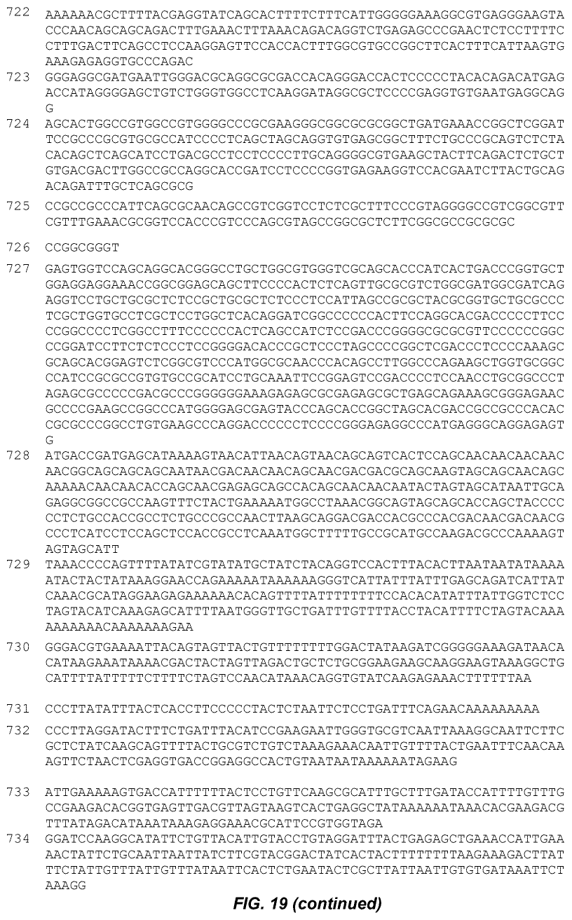

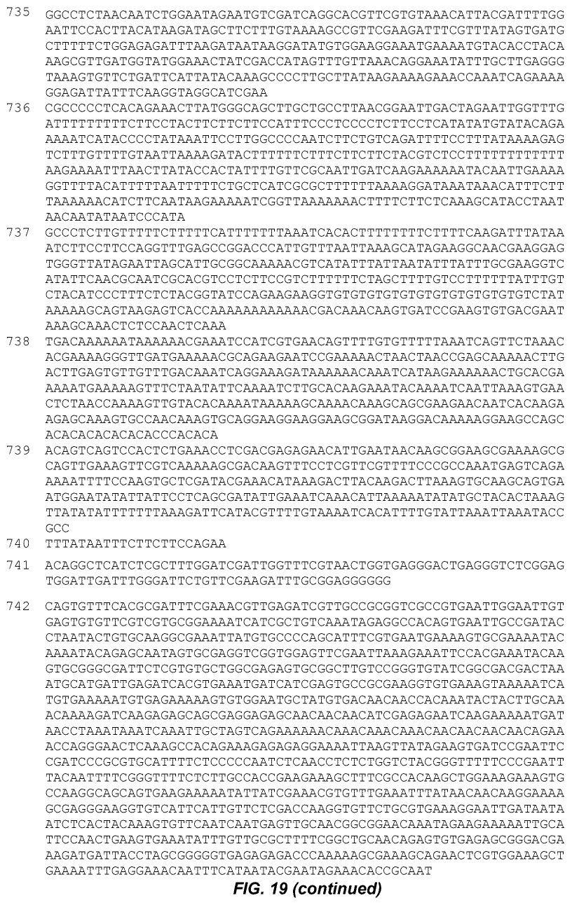

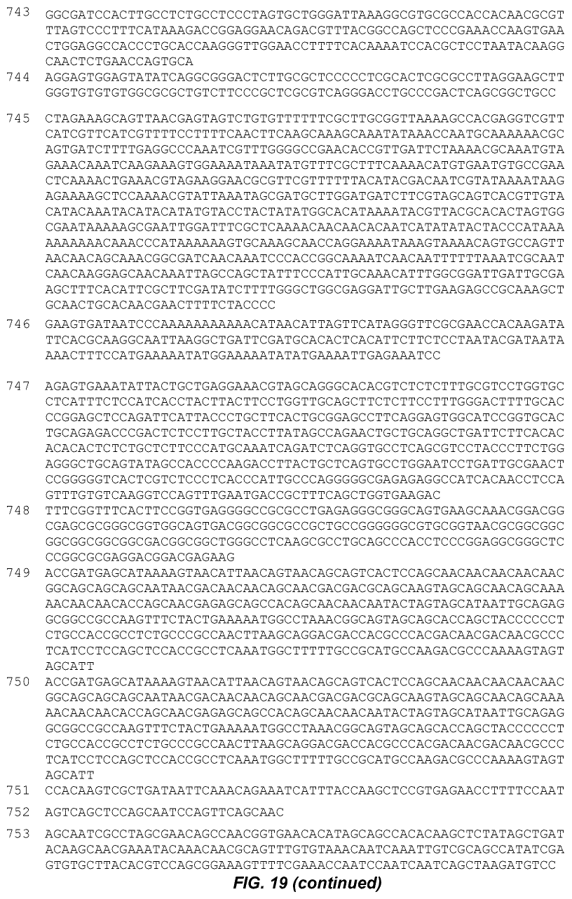

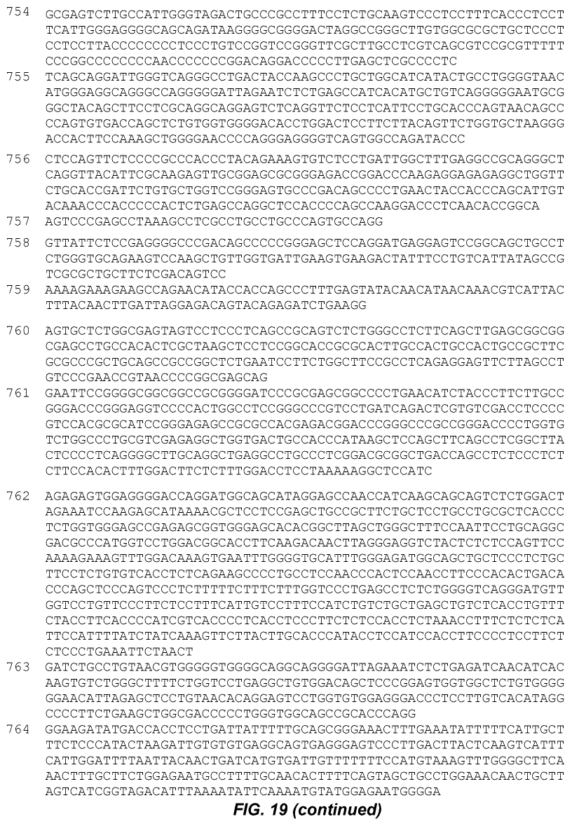

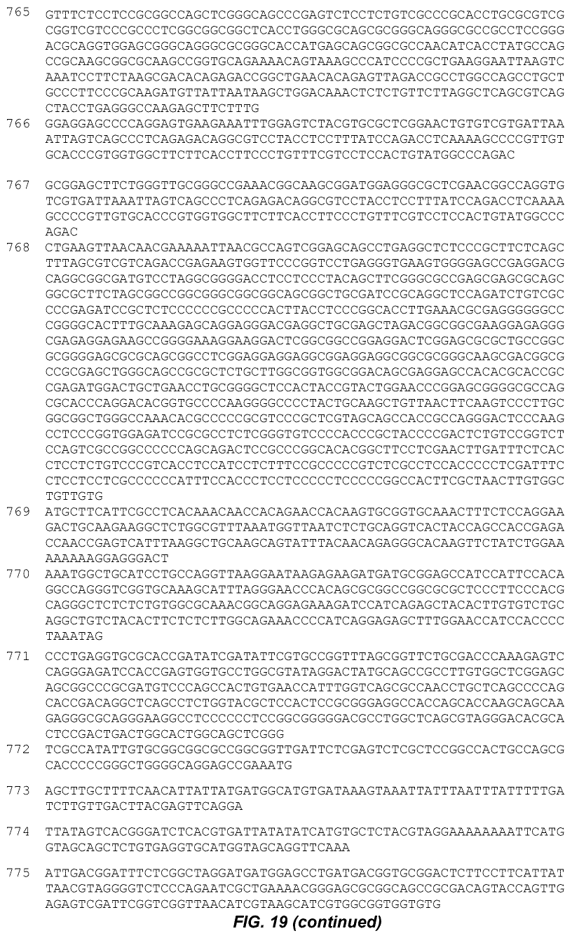

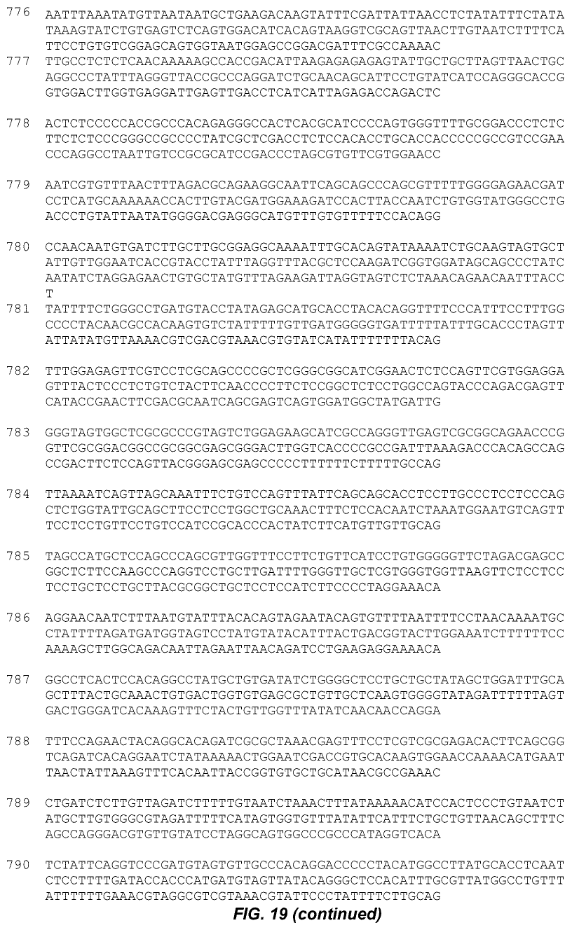

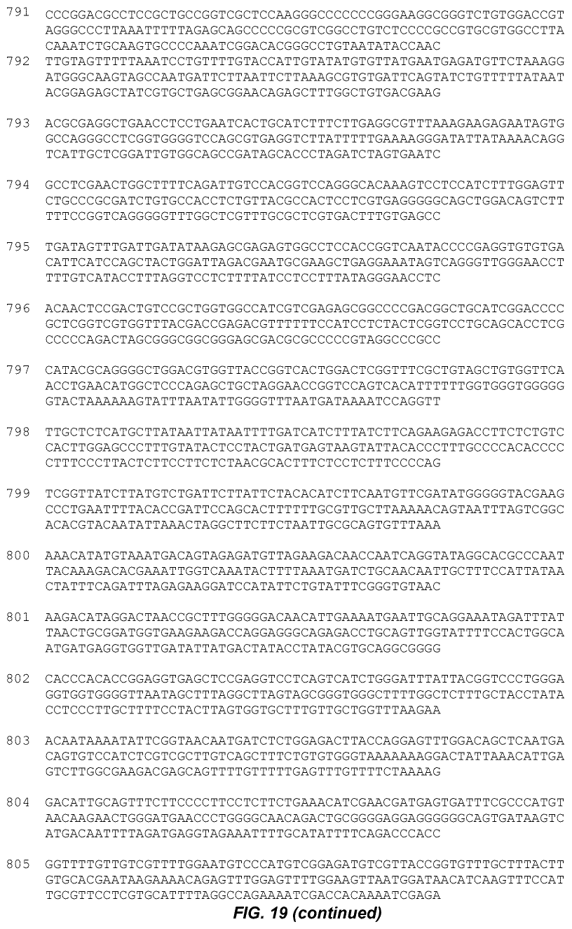

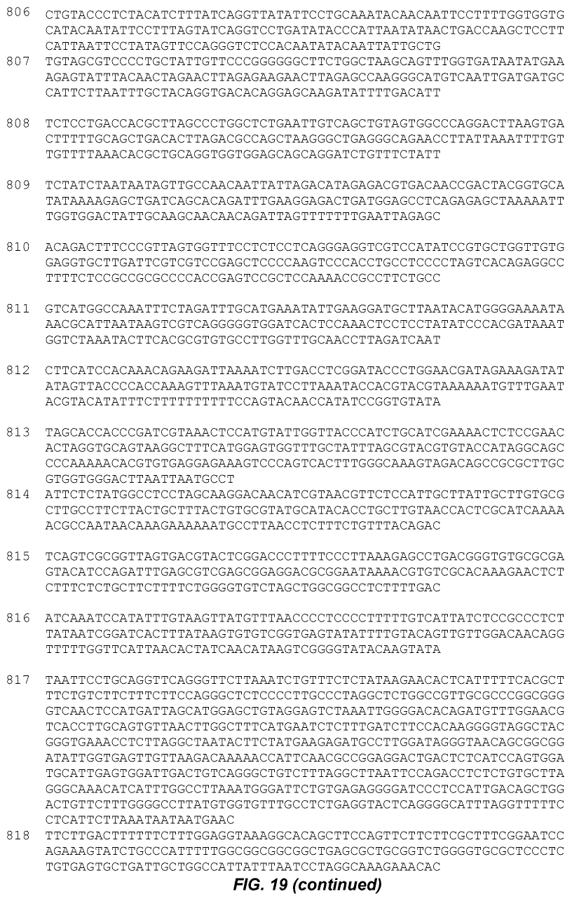

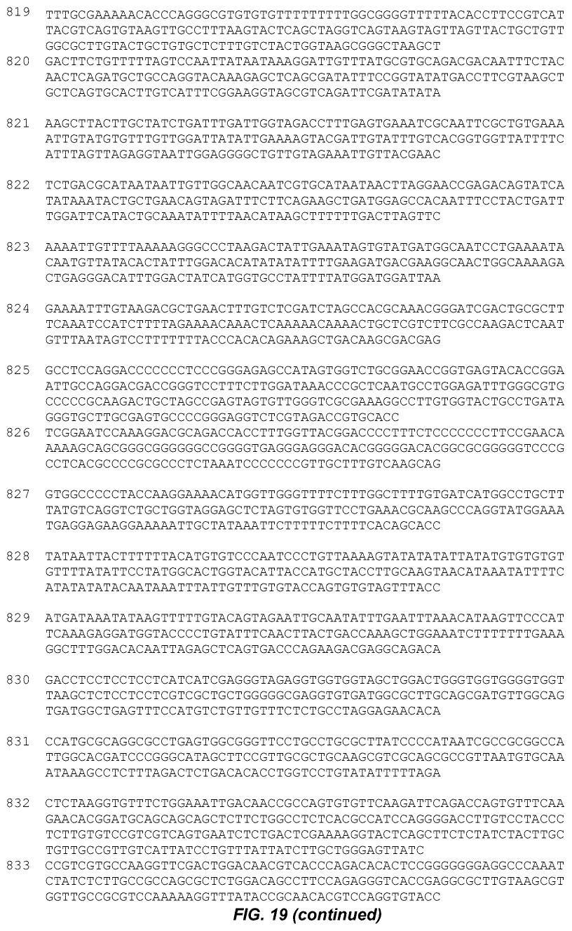

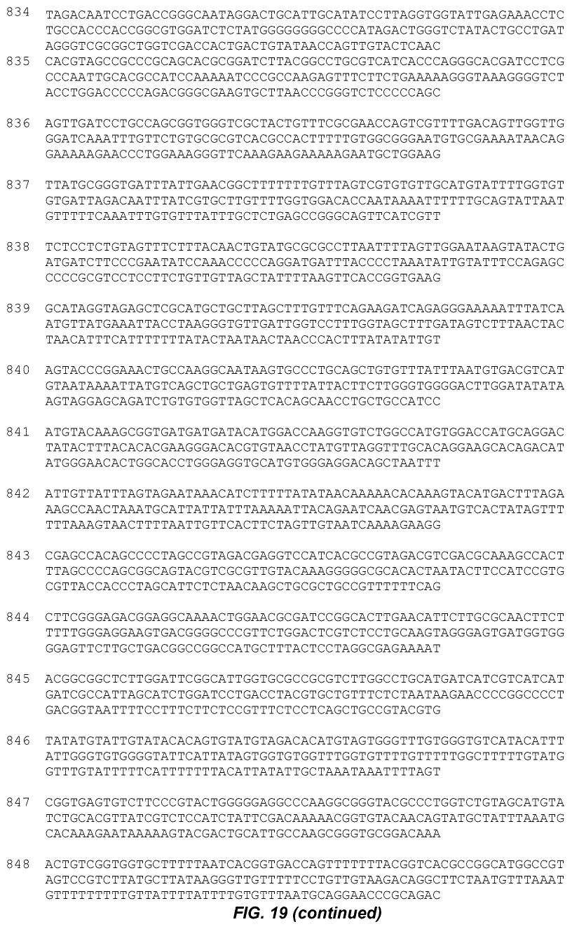

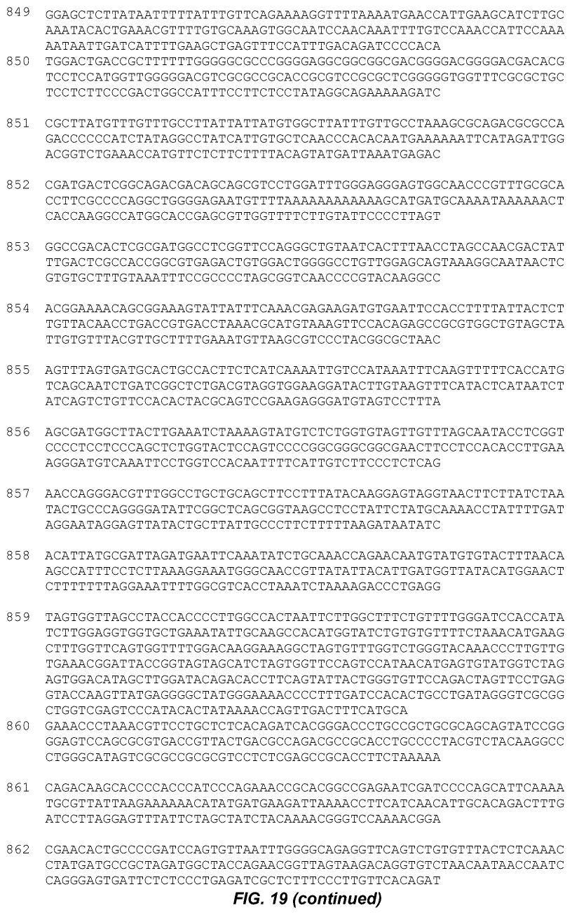

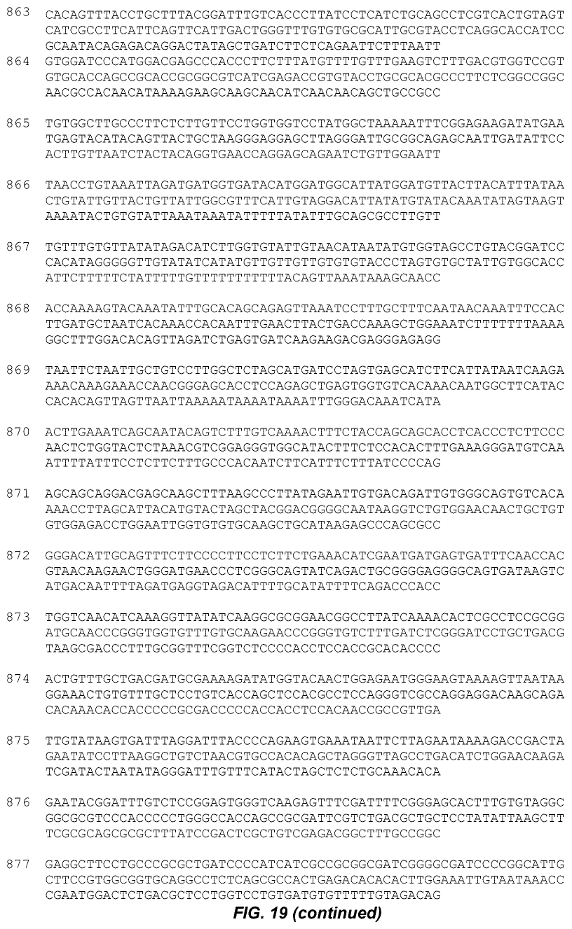

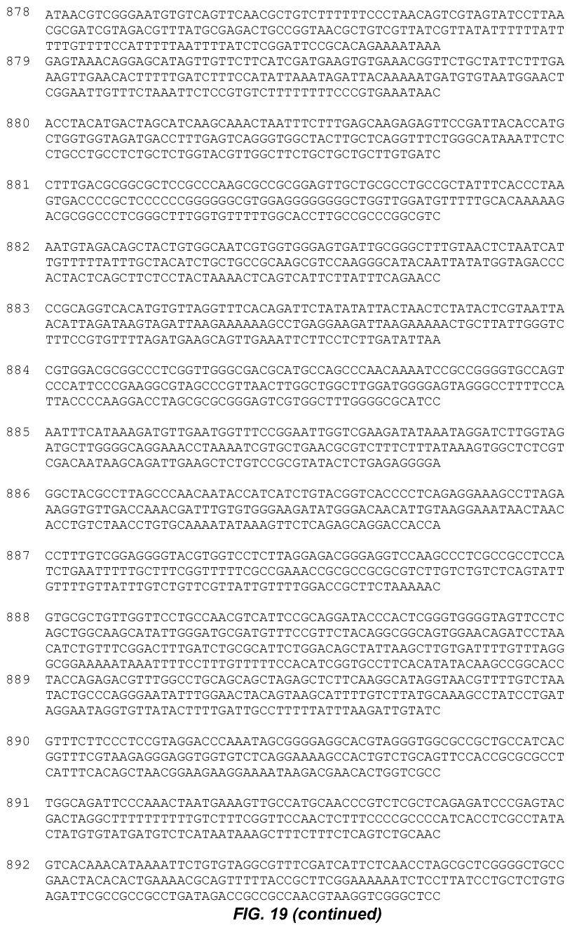

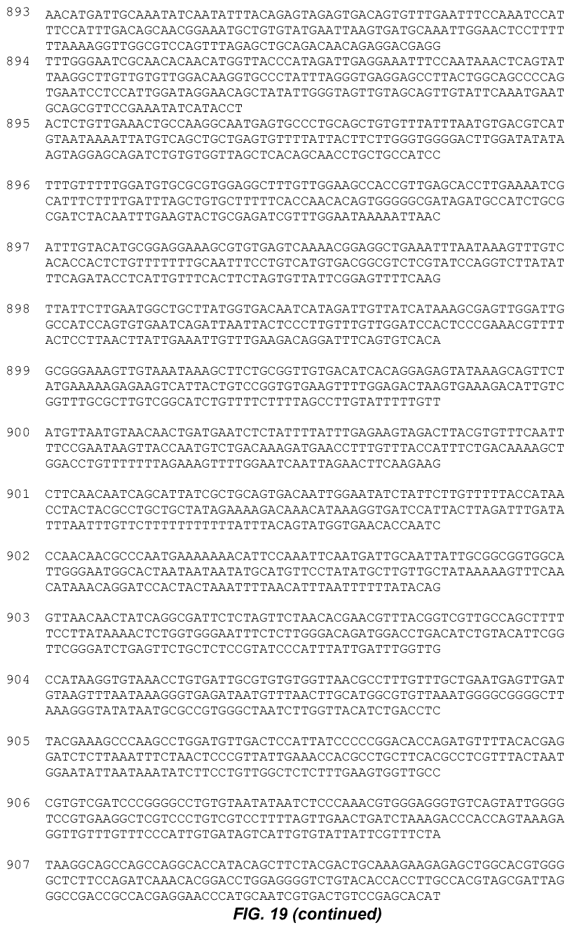

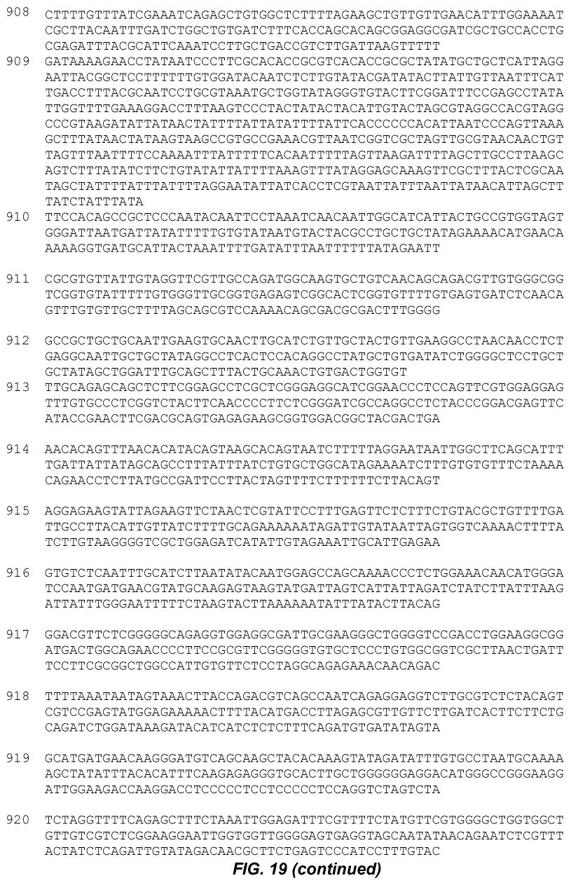

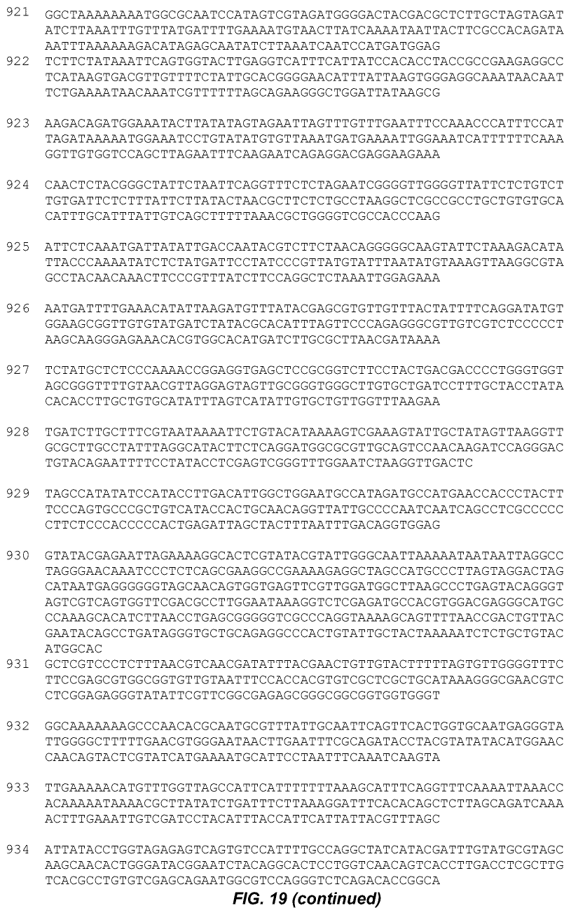

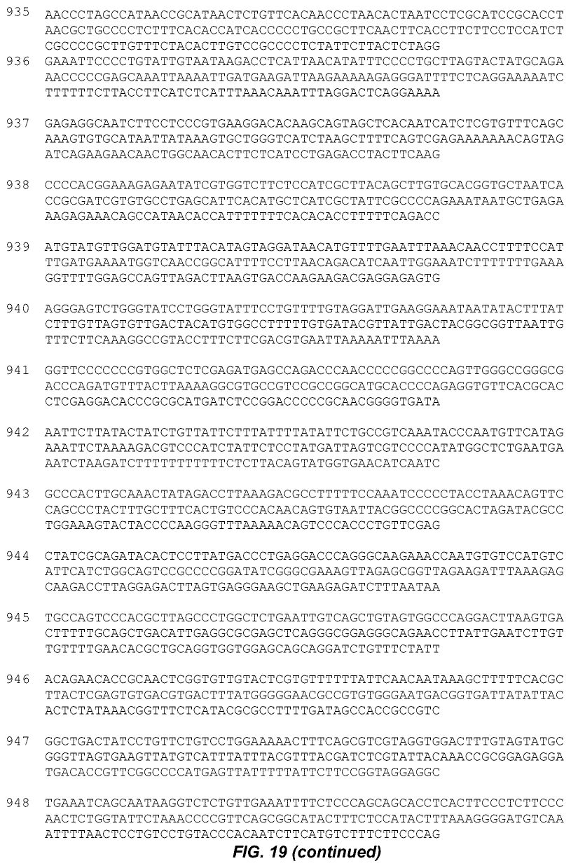

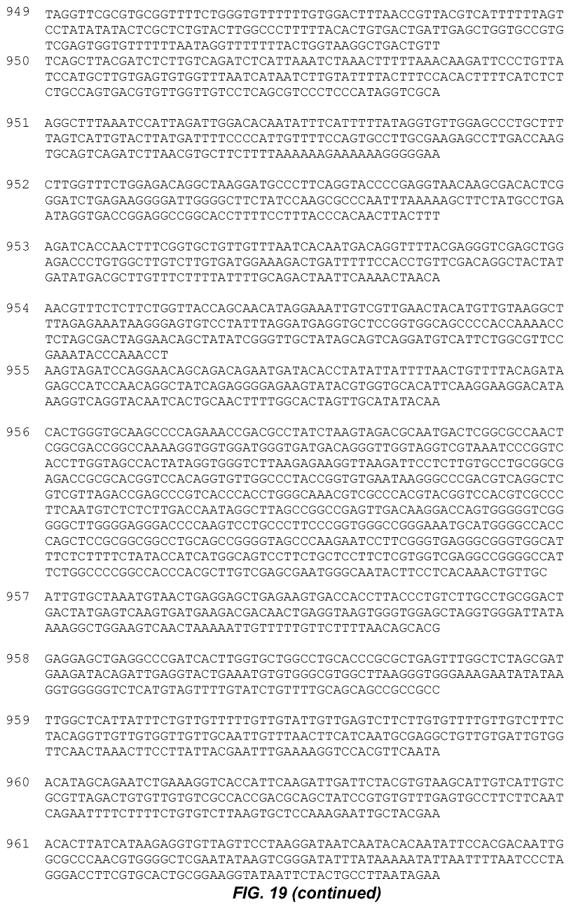

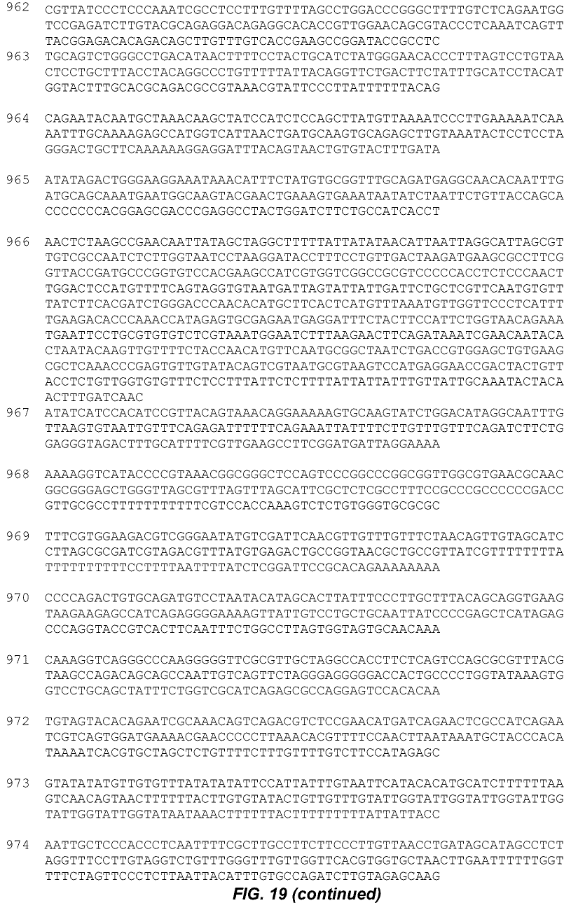

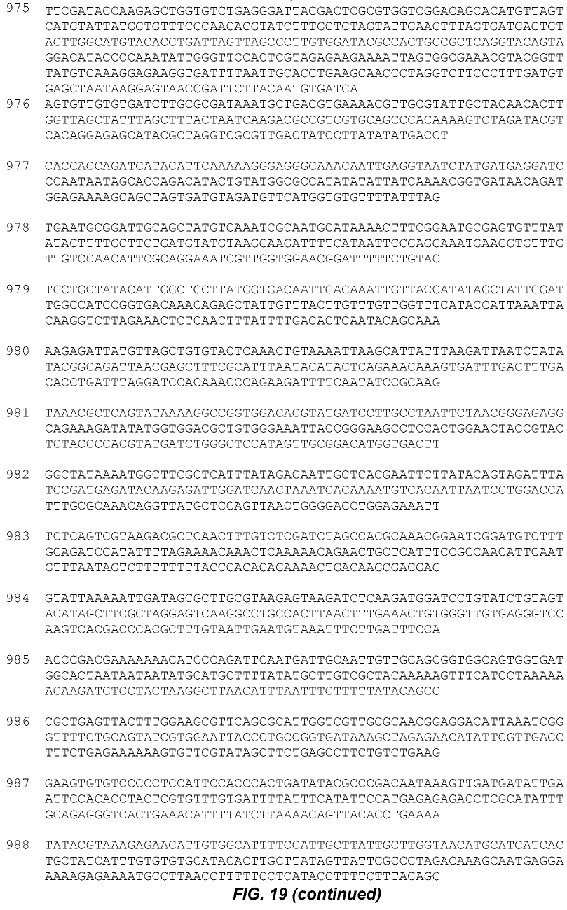

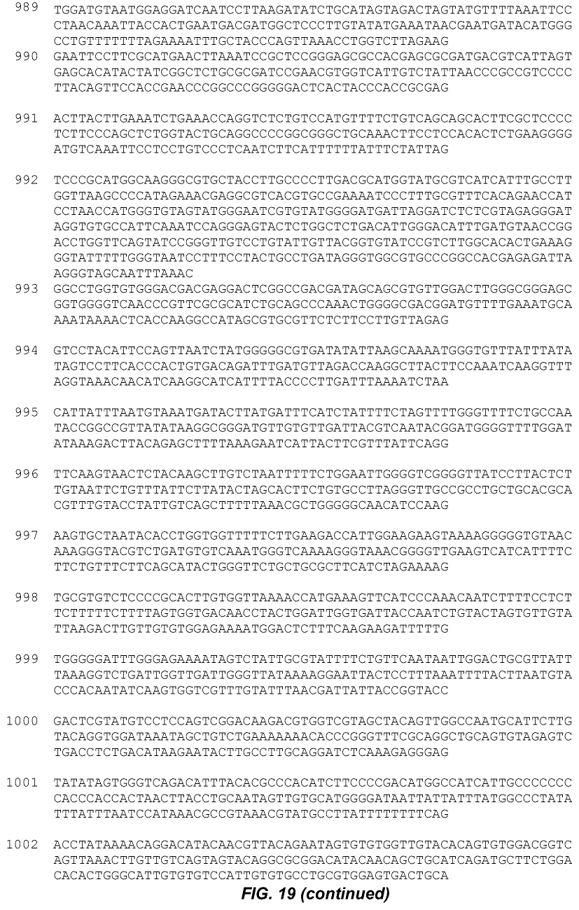

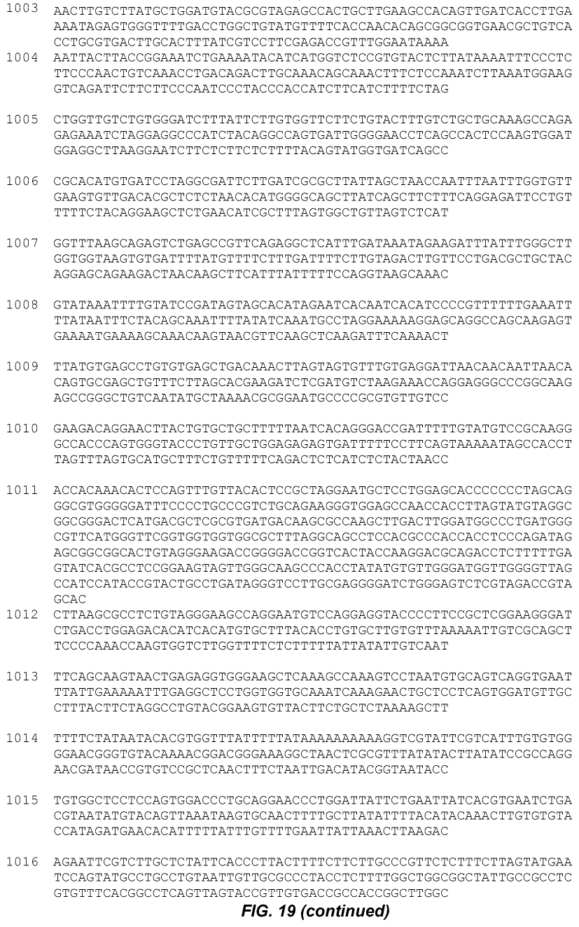

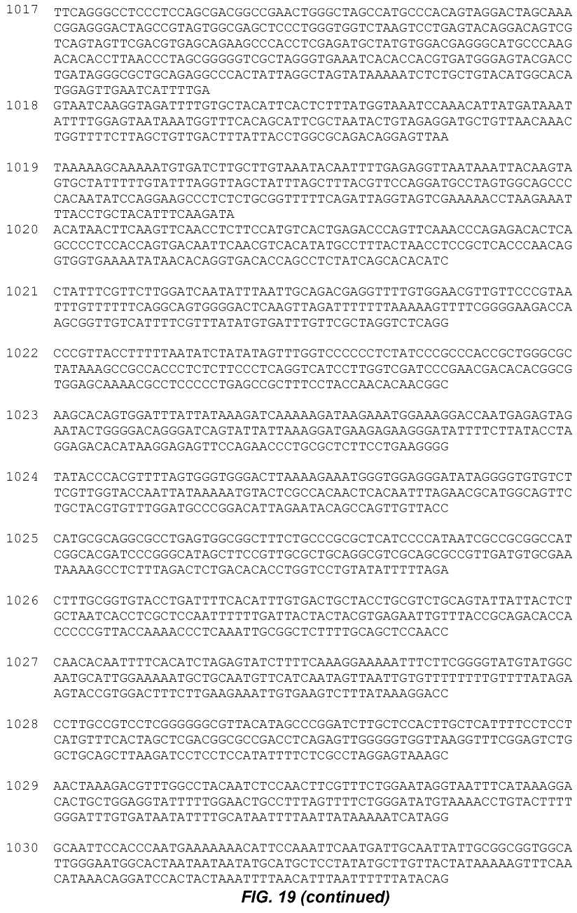

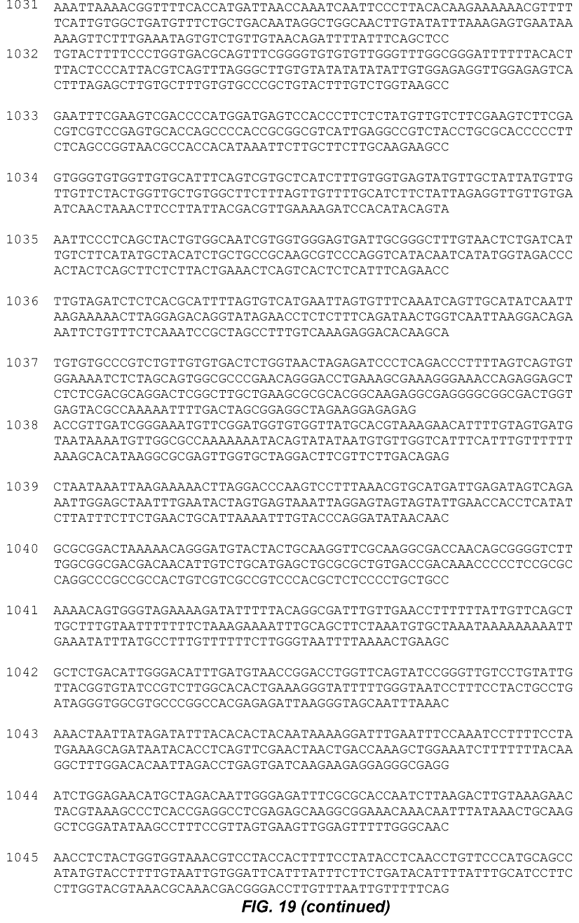

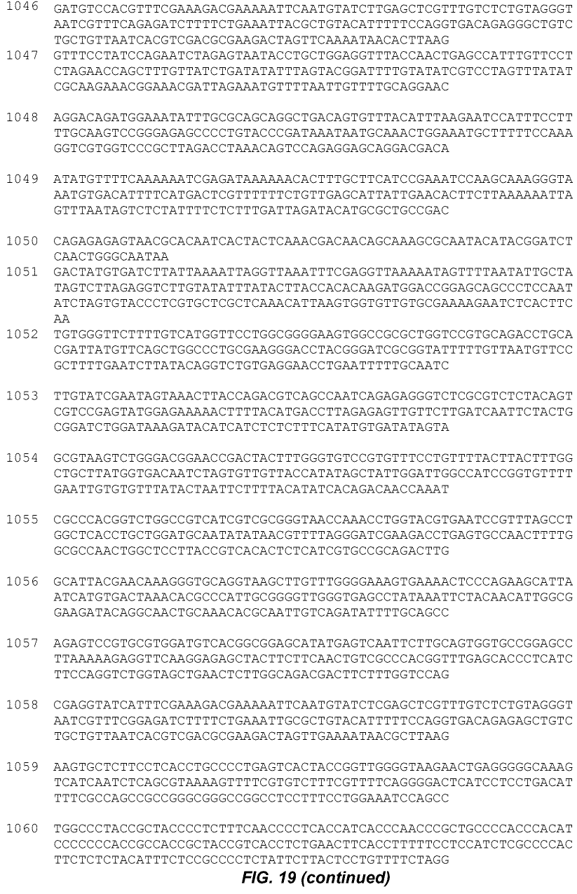

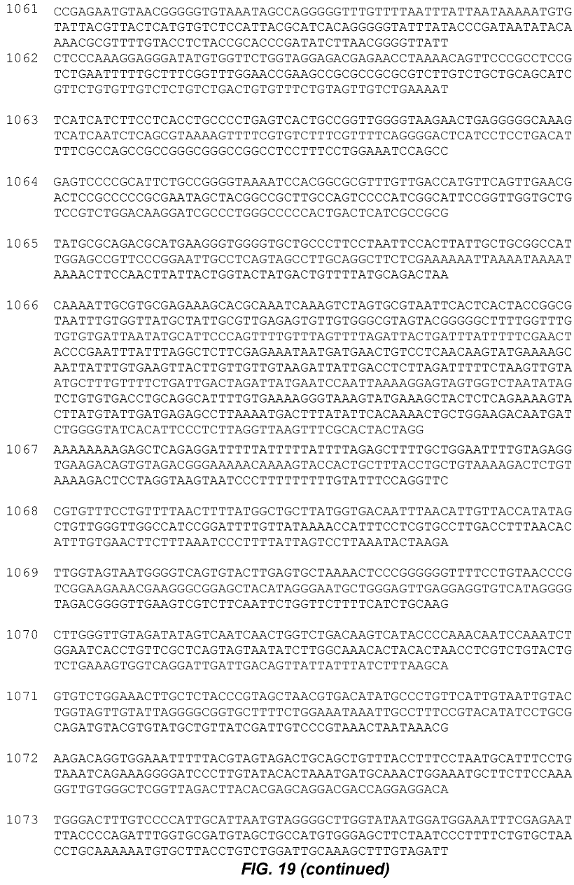

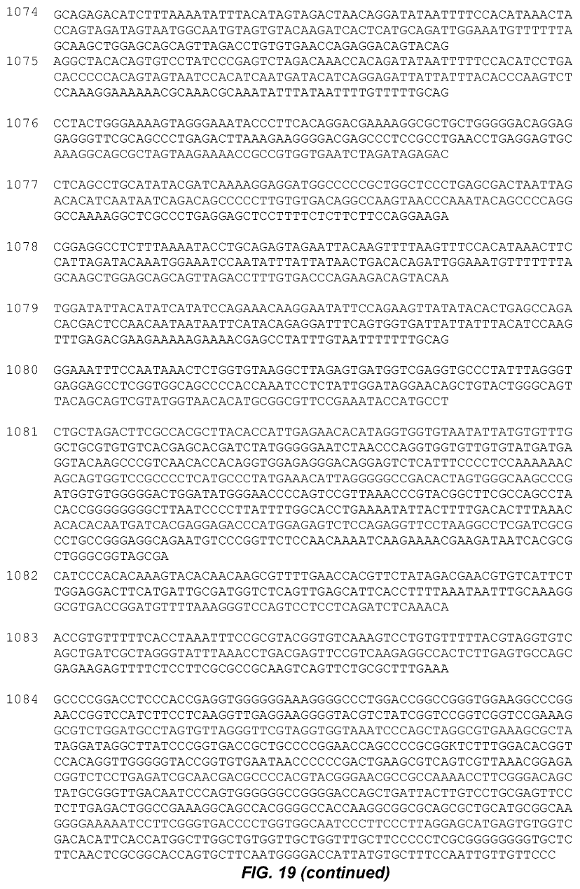

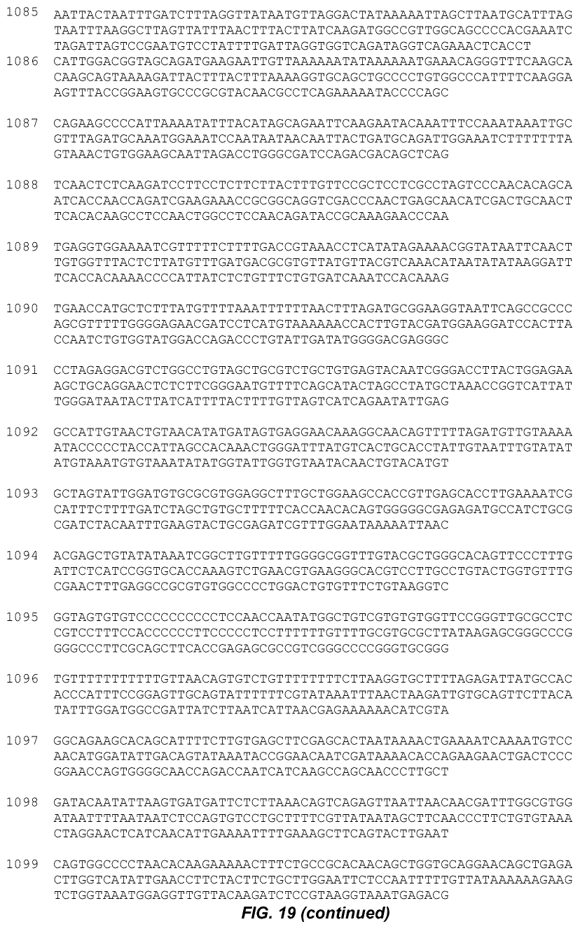

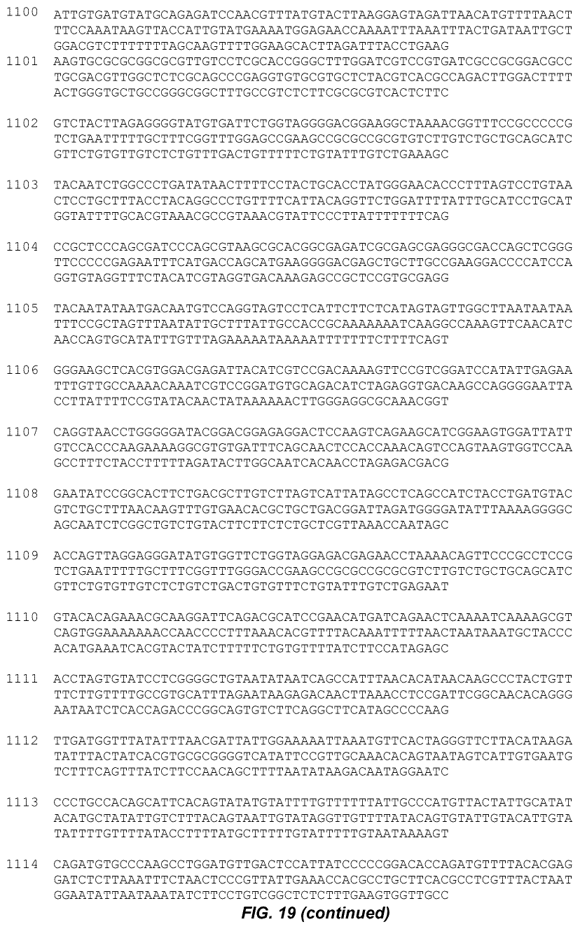

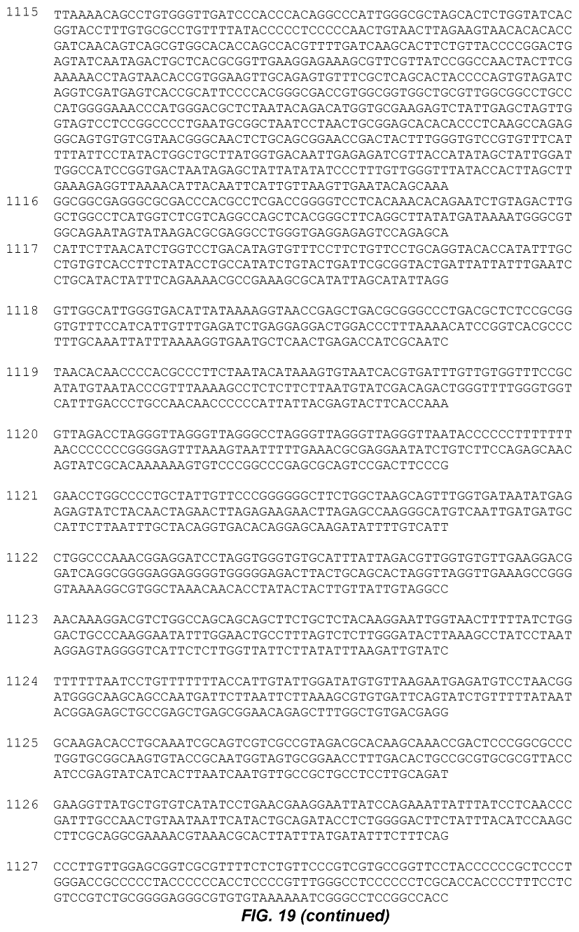

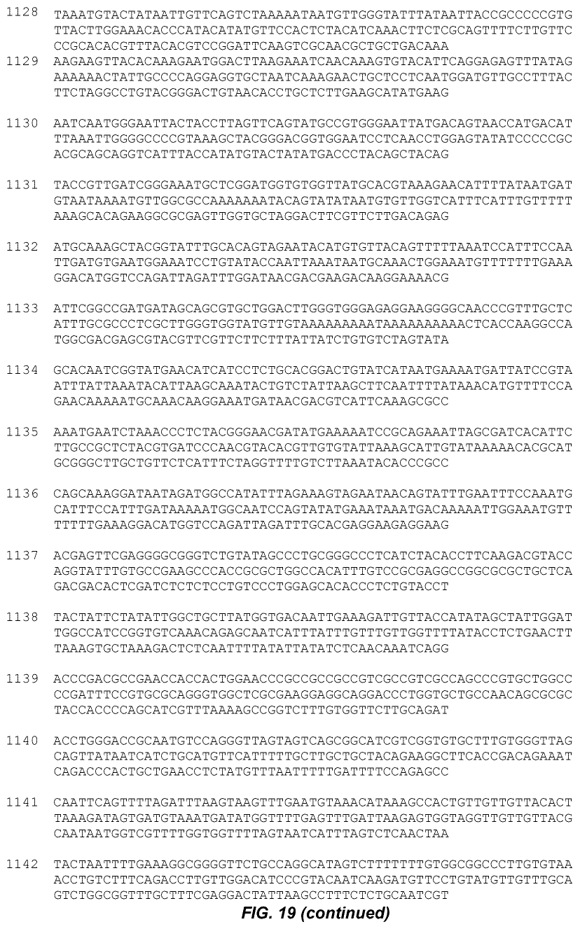

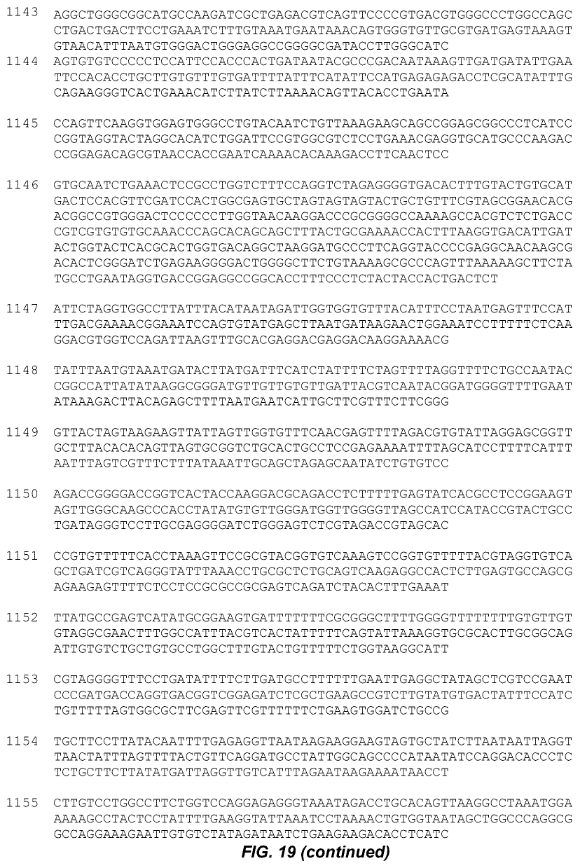

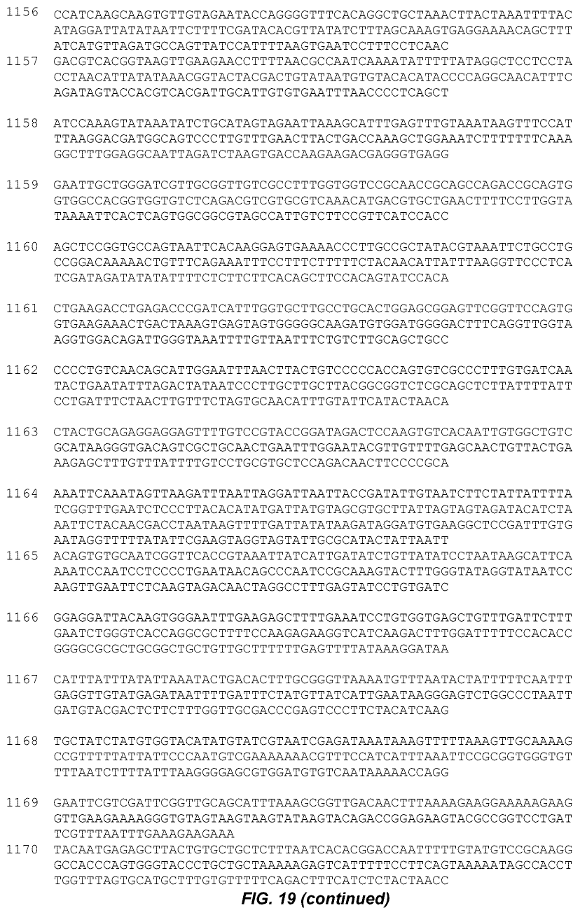

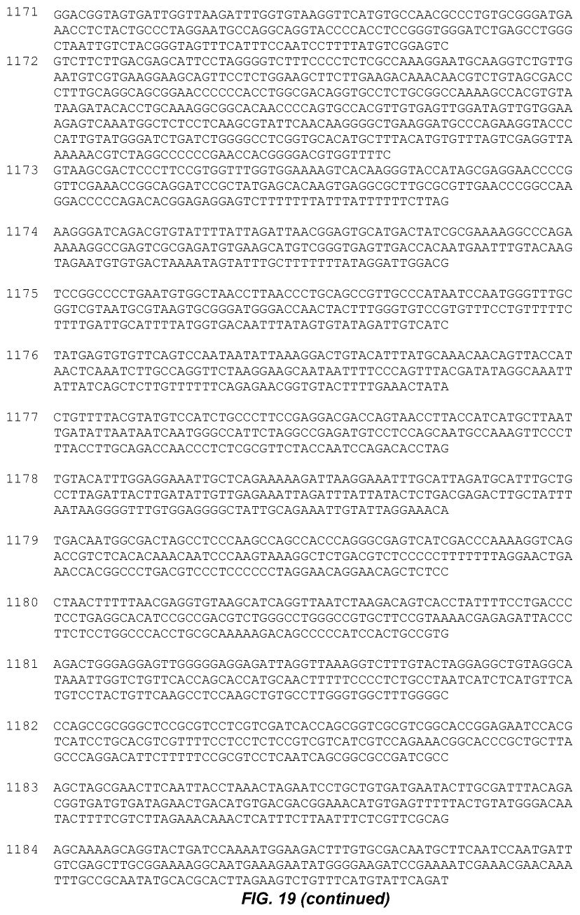

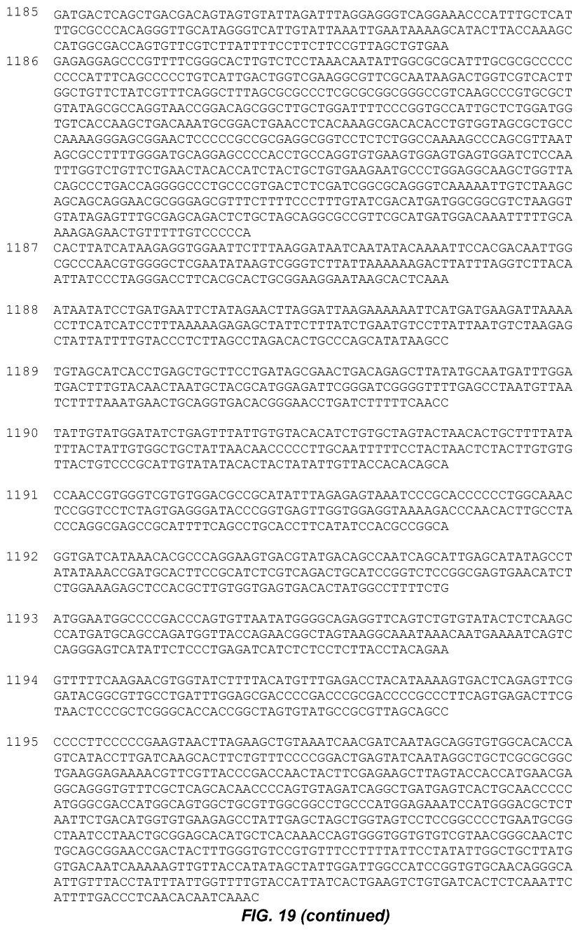

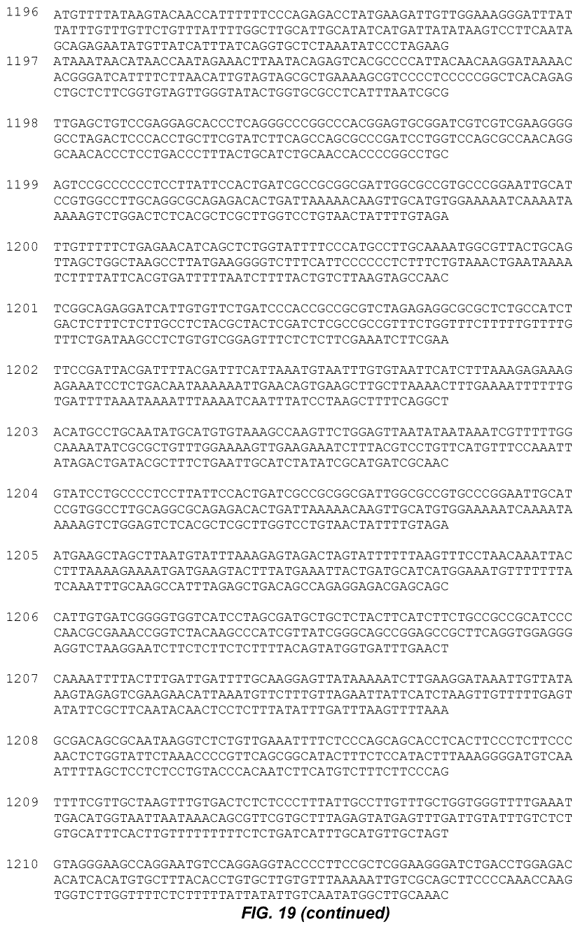

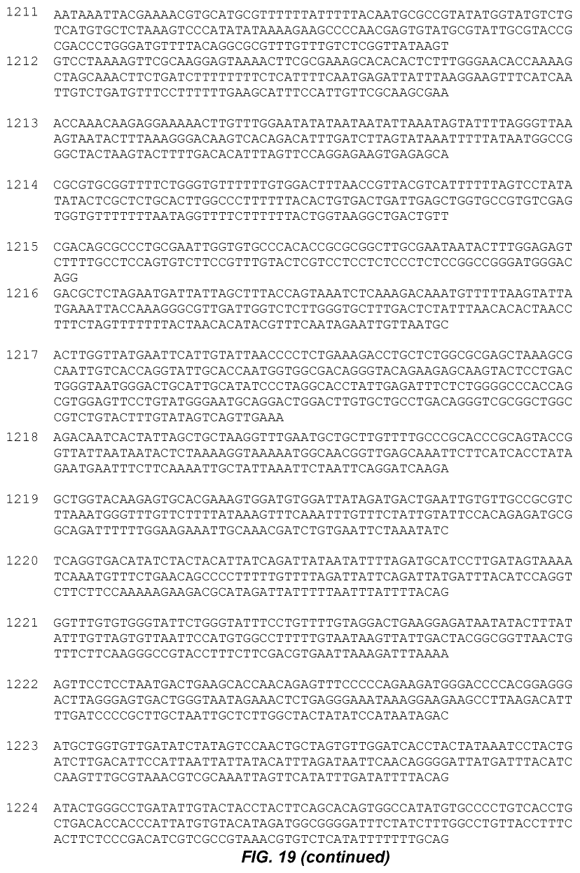

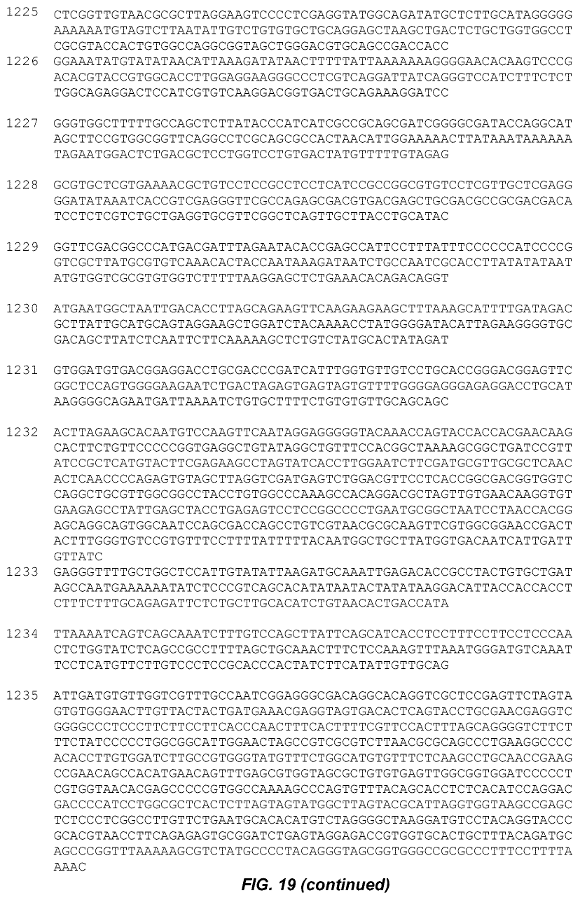

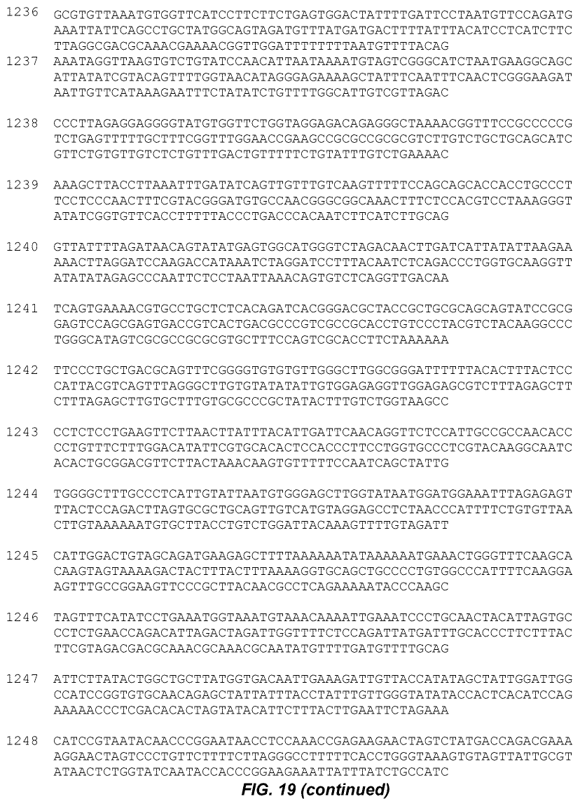

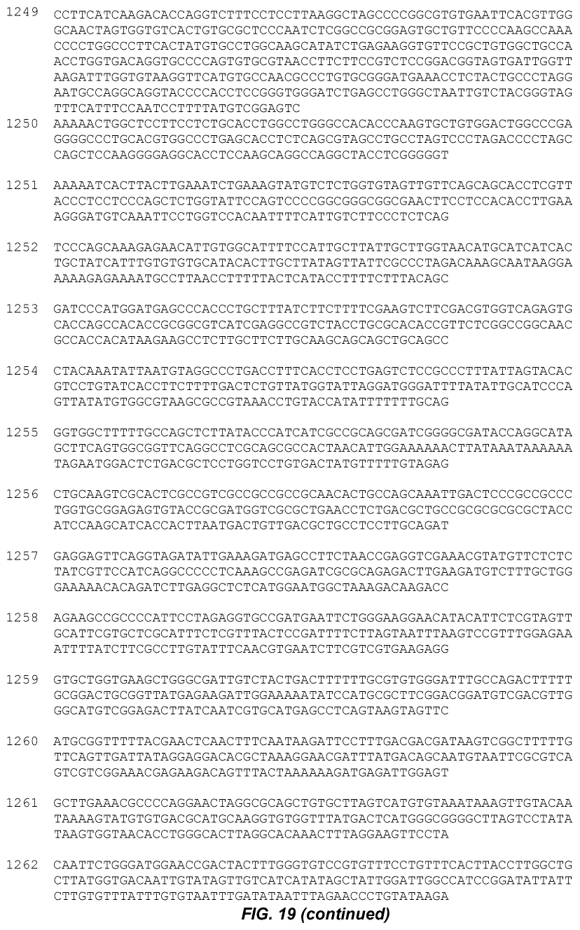

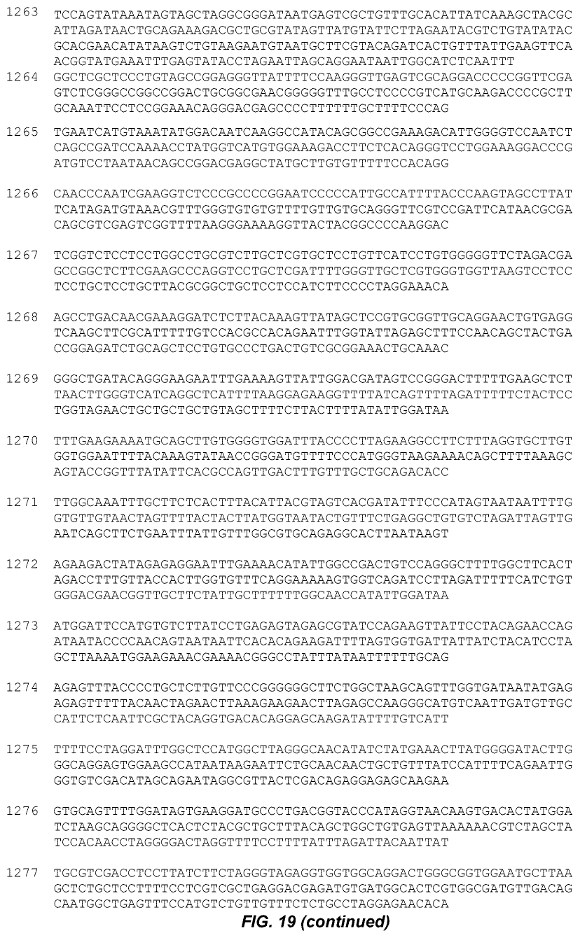

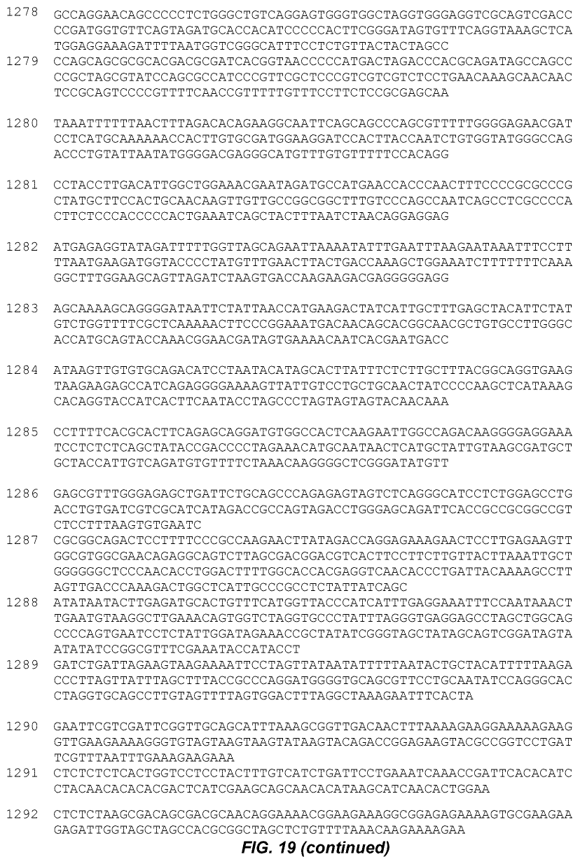

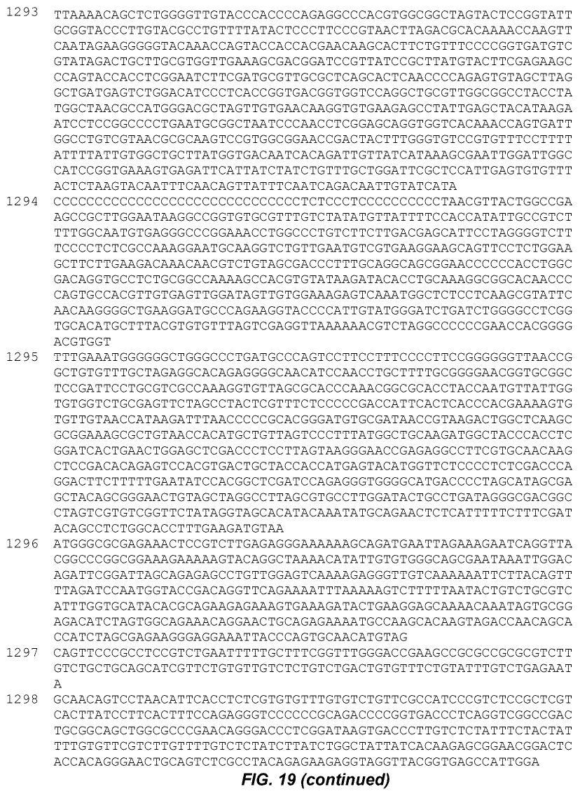

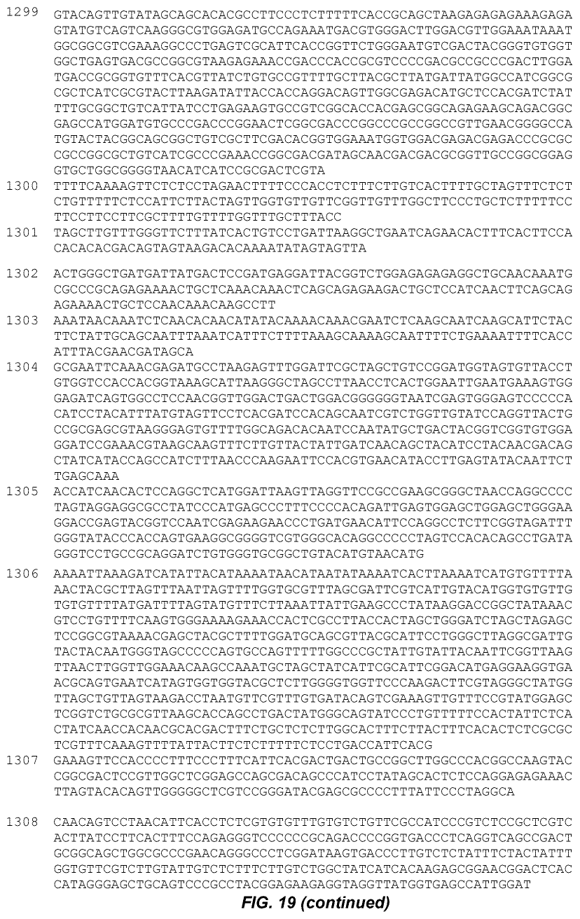

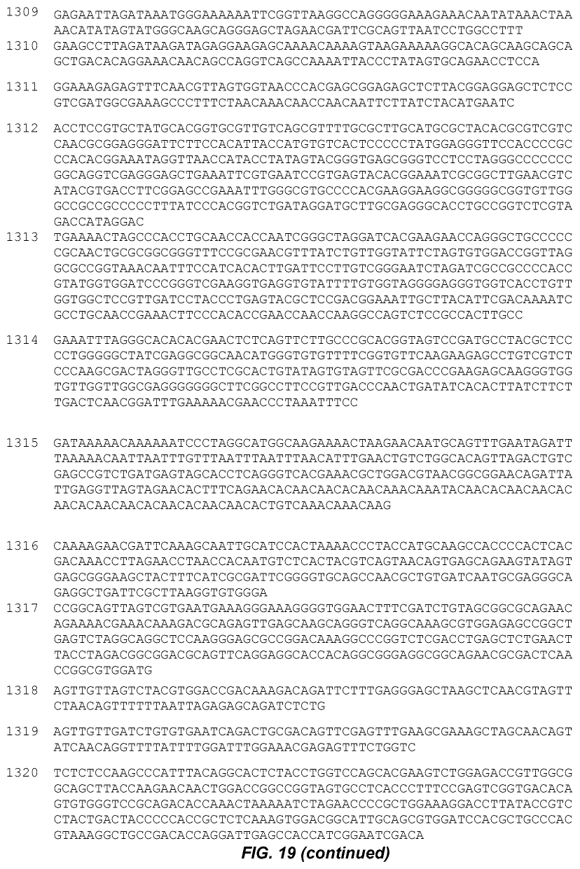

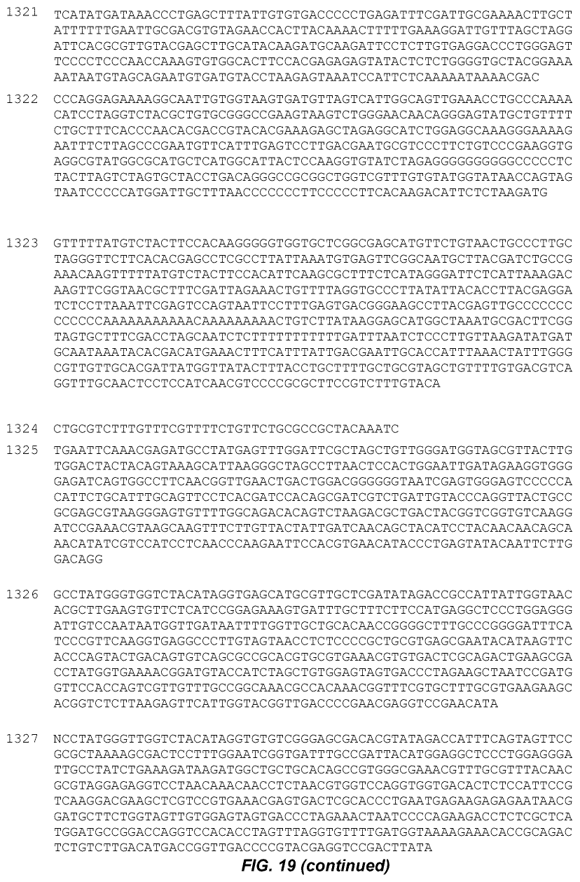

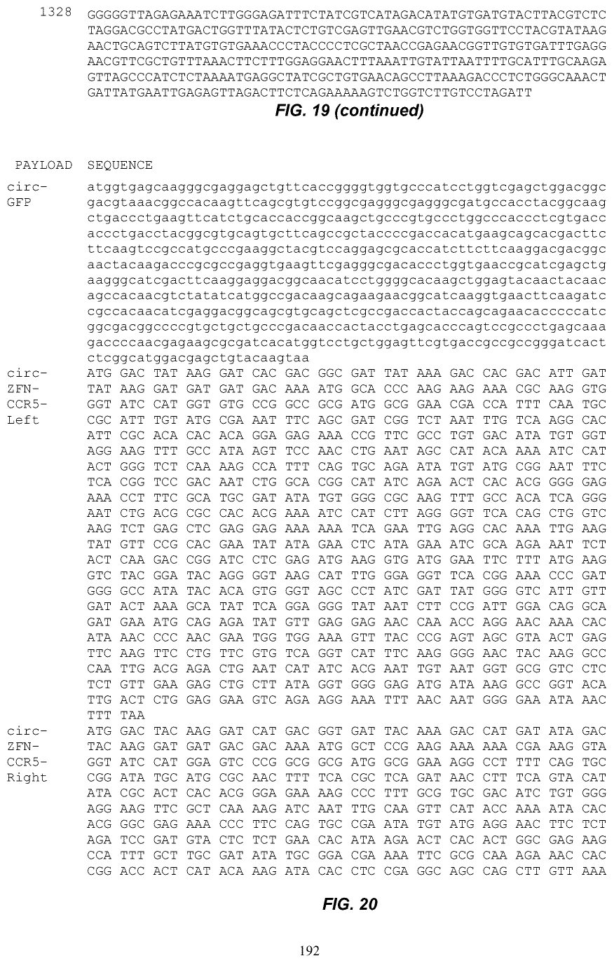

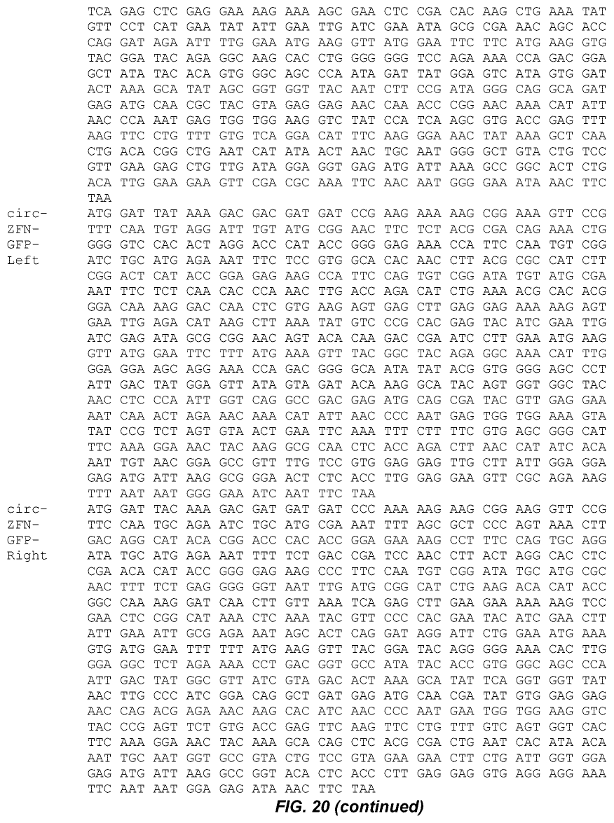

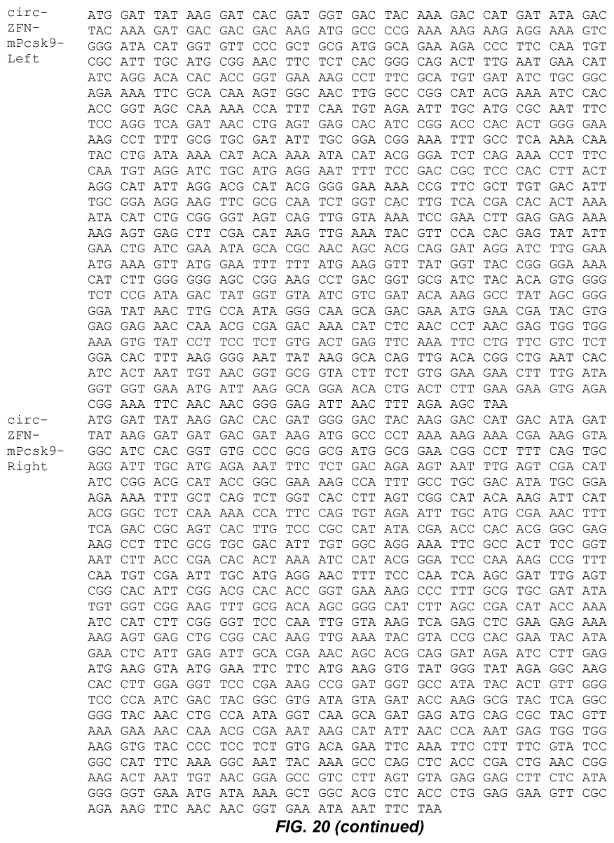

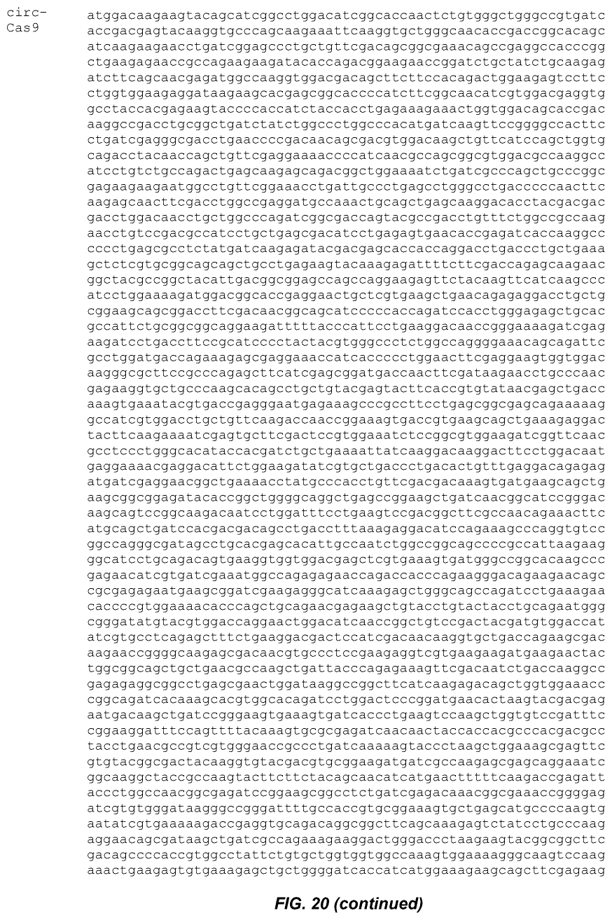

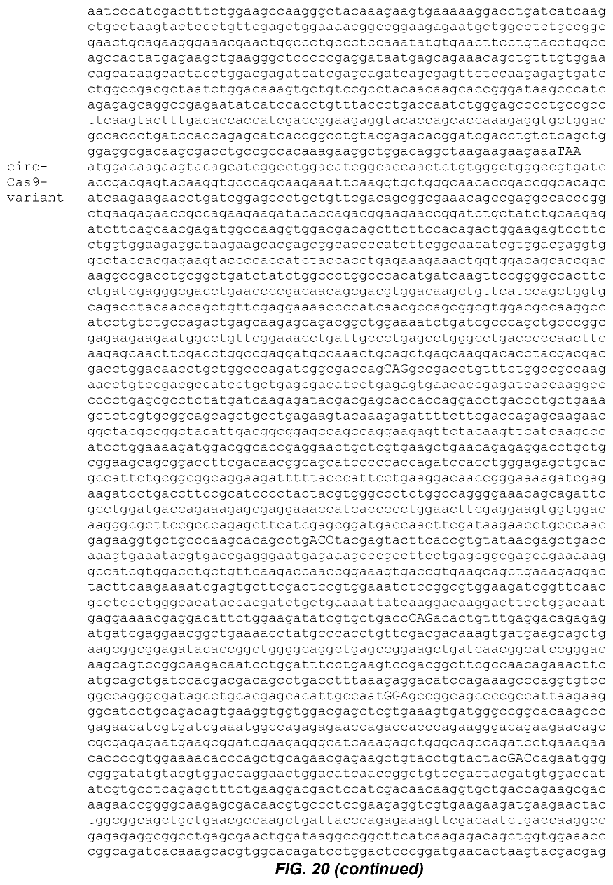

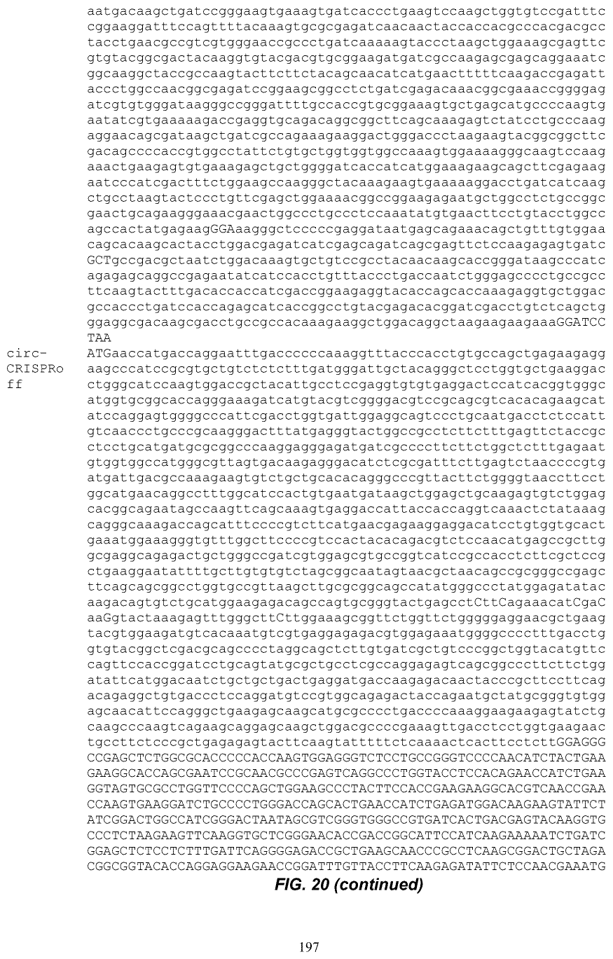

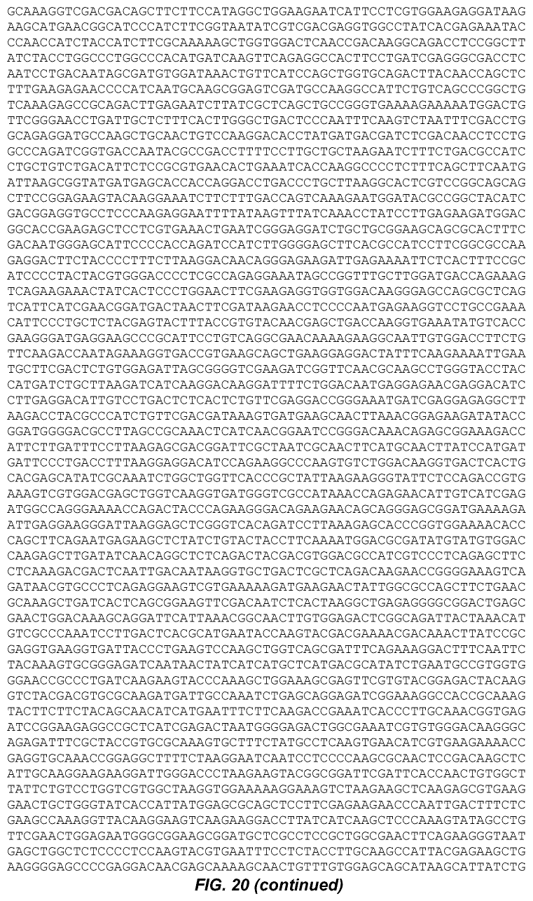

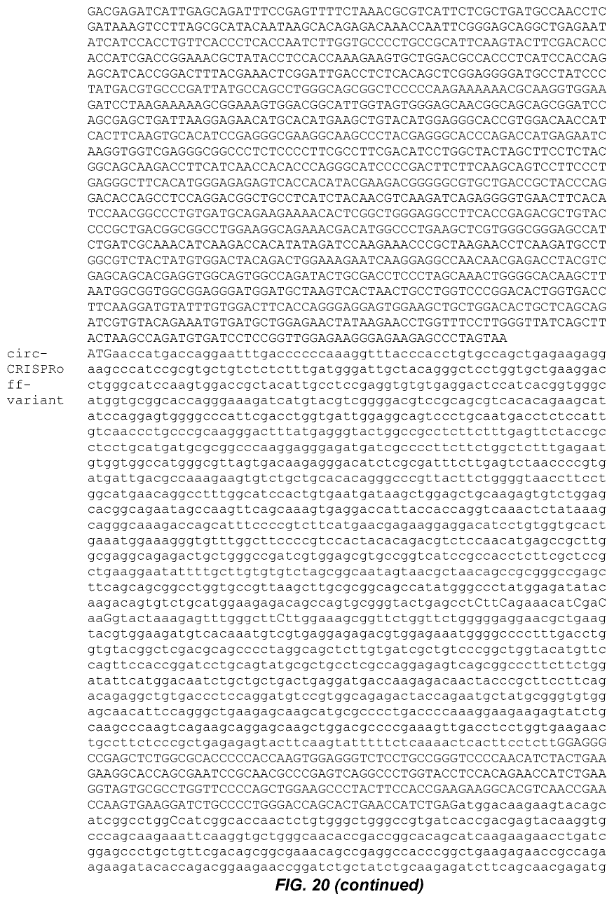

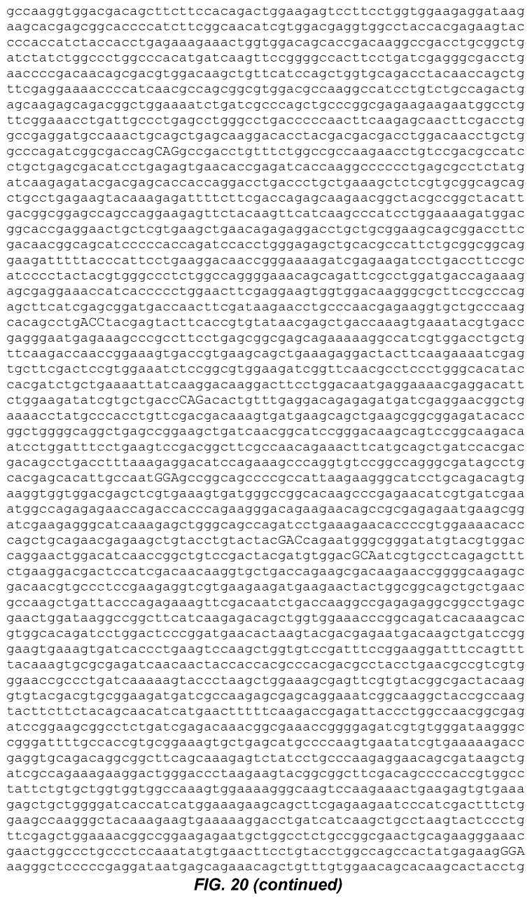

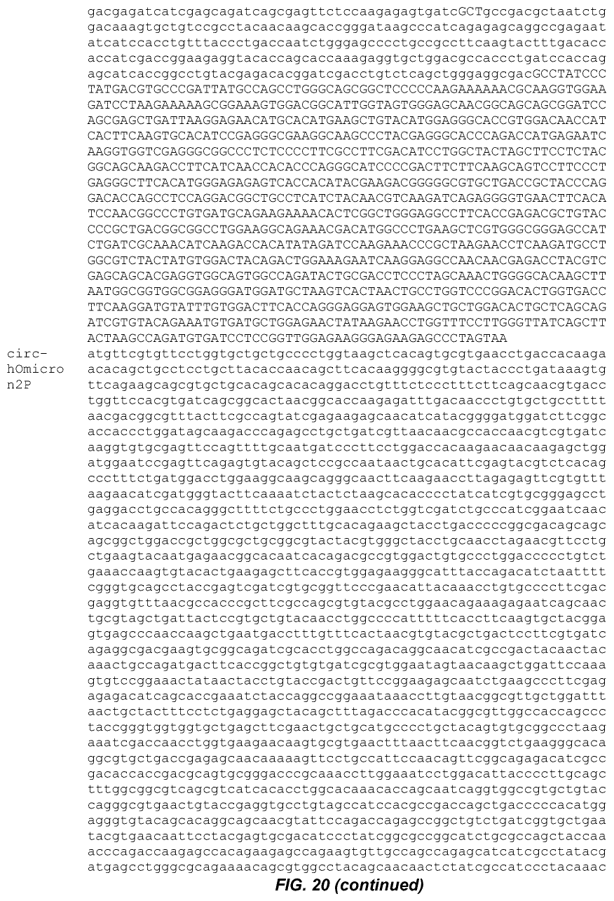

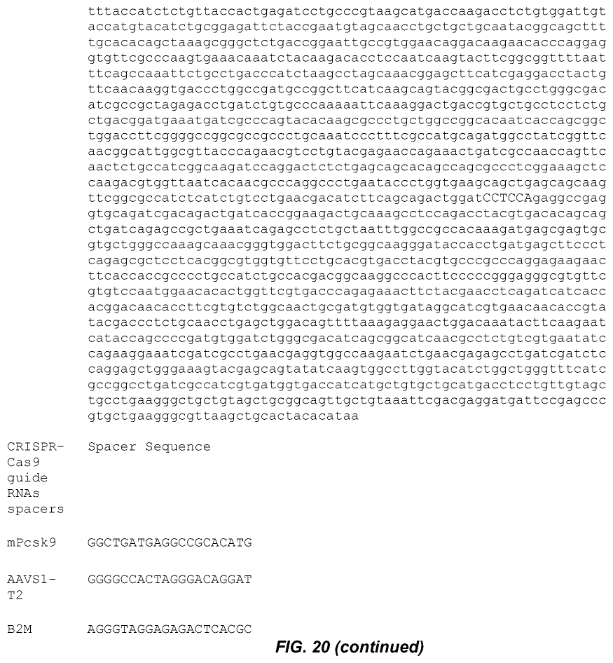

To generate a circular format for vaccines, a self-amplifying RNA system such as those associated with alpha-viruses was used downstream of the IRES. (see FIG. 18A-B).

It will be understood that various modifications may be made without departing from the spirit and scope of this disclosure. Accordingly, other embodiments are within the scope of the following claims.

1. A ribozyme activated RNA-construct(s) comprising:
one or more ribozymes; and
one or more RNA coding sequences for at least one polypeptide of interest;
wherein the transcription of the one or more RNA coding sequences for at least one polypeptide of interest is activated by or dependent upon the activity of the one or more ribozymes.

2. The ribozyme activated RNA-construct(s) of claim 1, further comprising:
a first engineered RNA element comprising an optional primer region, an optional barcode region, an RNA coding sequence for a polypeptide of interest and a complementary sequence to a sequence of a second engineered RNA element, and a first self-cleaving ribozyme;
a second engineered RNA element comprising an optional primer region, an optional barcode region, an RNA coding sequence for a polypeptide of interest and a complementary sequence to a sequence of the first engineered RNA element, and a second self-cleaving ribozyme;
wherein cleavage of the first and second engineered RNA elements by the first and second self-cleaving ribozymes, respectively, provides for a hybridization construct that comprises a region of dsRNA from the commentary sequences being hybridized together,
wherein the hybridization construct can be further ligated by an RNA ligase to form an RNA-fusion construct, and
wherein expression from the RNA-fusion construct produces the at least one polypeptide of interest.

3. The ribozyme activated RNA-construct(s) of claim 2, wherein the first engineered RNA element comprises a barcode sequence or a unique molecular identity (UMI) sequence and/or wherein the second engineered RNA element comprises a barcode sequence or a UMI sequence.

4. The ribozyme activated RNA-construct(s) of claim 3, wherein the barcode sequence or the UMI sequence of the first engineered element has a different sequence than the barcode region or the UMI sequence from the second engineered RNA element.

5. The ribozyme activated RNA-construct(s) of claim 2, wherein the first engineered RNA element comprises a primer sequence, and/or wherein the second engineered RNA element comprises a primer sequence.

6. The ribozyme activated RNA-construct(s) of claim 5, wherein the primer sequence of the first engineered RNA element is different from the primer sequence from the second engineered RNA element.

7. The ribozyme activated RNA-construct(s) of claim 2, wherein the first and second complementary sequences are from 30 to 60 bp in length.

8. (canceled)

9. The ribozyme activated RNA-construct(s) of claim 2, wherein the first and second ribozymes are Twister ribozymes.

10. The ribozyme activated RNA-construct(s) of claim 9, wherein the first ribozyme is a P3 Twister ribozyme.

11. The ribozyme activated RNA-construct(s) of claim 10, wherein the second ribozyme is a P1 Twister ribozyme.

12. The ribozyme activated RNA-construct(s) of claim 2, wherein the RNA ligase is RtcB.

13. The ribozyme activated RNA-construct(s) of claim 2, wherein a vector or plasmid comprises the first engineered element, wherein the first engineered element is located downstream of a first RNA promoter and a first perturbation element; and/or wherein a vector or plasmid comprises the second engineered element, wherein the second engineered element is located downstream of a second RNA promoter and a second perturbation element.

14. The ribozyme activated RNA-construct(s) of claim 13, wherein the first RNA promoter and/or the second RNA promoter is a polymerase III promoter.

15. (canceled)

16. The ribozyme activated RNA-construct(s) of claim 13, wherein the first perturbation element and/or the second perturbation element is a sgRNA utilized in a CRISPR knockout screen.

17. (canceled)

18. The ribozyme activated RNA-construct(s) of claim 1, comprising:
a first engineered RNA element comprising an RNA coding sequence for a polypeptide of interest, an intron sequence, a complementary sequence to a sequence of a second engineered RNA element and a 3′ aptamer, and a first self-cleaving ribozyme, and wherein the 3′ aptamer interacts with a first self-cleaving ribozyme to stabilize it;
a second engineered RNA element comprising an RNA coding sequence for a polypeptide of interest, an intron sequence, a complementary sequence to a sequence of the first engineered RNA element, and a 3′ aptamer, wherein the second engineered RNA template is tethered to a second self-cleaving ribozyme, and wherein the 3′ aptamer interacts with a second self-cleaving ribozyme to stabilize it;
wherein cleavage of the first and second engineered RNA elements by the first and second self-cleaving ribozymes, respectively, provides for a hybridization construct that comprises a region of dsRNA from the commentary sequences being hybridized together, and
wherein the hybridization construct can be further ligated by an RNA ligase and the intron sequences removed by a spliceosome to form an RNA-fusion construct,
wherein expression from the RNA-fusion construct produces the at least one polypeptide of interest.

19. The ribozyme activated RNA-construct(s) of claim 18, wherein the intron sequence is derived from dihydrofolate reductase.

20. The ribozyme activated RNA-construct(s) of claim 18, wherein the RNA coding sequences for a polypeptide of interest are adjacent to each of the intron sequences.

21. The ribozyme activated RNA-construct(s) of claim 18, wherein the RNA coding sequences for a polypeptide of interest encode a polypeptide/protein selected from insulin, clotting factor IX, the cystic fibrosis transmembrane conductance regulator protein, and the dystrophin protein.

22. A pharmaceutical composition comprising the ribozyme activated RNA-construct(s) of claim 1, wherein the ribozyme activated RNA-construct(s) is linearized and comprises:
a 5′ ribozyme;
a 5′ ligation sequence;
an internal ribosome entry site (IRES) sequence;
an RNA coding sequence for at least one polypeptide of interest;
a 3′ ligation sequence; and
a 3′ ribozyme sequence, and
a pharmaceutically acceptable carrier.

23. The pharmaceutical composition of claim 22, wherein the linear ribozyme activated RNA-construct(s) lacks a polymerase binding region.

24. The pharmaceutical composition of claim 22, wherein the 5′ and 3′ ribozymes are selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing.

25. The pharmaceutical composition of claim 22, wherein the 5′ and 3′ ligation sequences are substrates of naturally occurring ligases in situ.

26. The pharmaceutical composition of claim 25, wherein the naturally occurring ligase is RtcB.

27. The pharmaceutical composition of claim 22, wherein the IRES comprises any one of the sequences of SEQ ID NO:1-1328.

28. The pharmaceutical composition of claim 22, wherein the at least one polypeptide of interest comprises two or more polypeptides of interest separated by a self-cleaving peptide.

29. The pharmaceutical composition of claim 28, wherein the self-cleaving peptide comprises a 2A- or 2A-like-peptide.

30. The pharmaceutical composition of claim 22, wherein the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

31. The pharmaceutical composition of claim 30, wherein the biological response modifier is an immunopotentiating cytokine.

32. The pharmaceutical composition of claim 31, wherein the immunopotentiating cytokine is selected from the group consisting of interleukins 1 through 38, interferon, tumor necrosis factor (TNF), and granulocyte-macrophage-colony stimulating factor (GM-CSF).

33. The pharmaceutical composition of claim 29, wherein the 2A- or 2A-like peptide further comprises a GSG linker moiety.

34. The pharmaceutical composition of claim 30, wherein the genome editing enzyme is selected from the group consisting of a zinc finger nuclease, a transcription activator-like effector nuclease (TALEN), an engineered meganuclease and an RNA-guided DNA endonuclease (Cas) polypeptide.

35. The pharmaceutical composition of claim 22, wherein the 5′ and 3′ ribozyme sequences are independently selected from a sequence that is at least 85-100% identical to 5′-GCCATCAGTCGCCGGTCCCAAGCCCGGATAAAATGGGAGGGGGGGGAAACC GCCT-3′ (SEQ ID NO:1354) or 5′-AACACTGCCAATGCCGGTCCCAAGCCCGGATAAAAGTGGAGGGTACAGTCCAC GC-3′ (SEQ ID NO:1355).

36. The pharmaceutical composition of claim 22, wherein the 5′ and 3′ ligation sequences are independently selected from a sequence that is at least 85-100% identical to 5′-AACCATGCCGACTGATGGCAG-3′ (SEQ ID NO: 1356) or 5′-CTGCCATCAGTCGGCGTGGACTGTAG-3′ (SEQ ID NO: 1357).

37. The pharmaceutical composition of claim 22, wherein the IRES sequence is at least 85-100% identical to 5′-gcggccgcgtcgacgggcccgcggaattccgccccccccccctctccctcccccccccctaacgttactggccgaa gccgcttggaataaggccggtgtgcgtttgtctatatgttattttccaccatattgccgtcttttggcaatgtgagggcccgg aaacctggccctgtcttcttgacgagcattcctaggggtctttcccctctcgccaaaggaatgcaaggtctgttgaatgtc gtgaaggaagcagttcctctggaagcttcttgaagacaaacaacgtctgtagcgaccctttgcaggcagcggaaccc cccacctggcgacaggtgcctctgcggccaaaagccacgtgtataagatacacctgcaaaggcggcacaacccc agtgccacgttgtgagttggatagttgtggaaagagtcaaatggctctcctcaagcgtattcaacaaggggctgaagg atgcccagaaggtaccccattgtatgggatctgatctggggcctcggtgcacatgctttacatgtgtttagtcgaggttaa aaaaacgtctaggccccccgaaccacggggacgtggttttcctttgaaaaacacgatgataatatggccacaacc-3′ (SEQ ID NO:1358).

38. A vaccine composition comprising the ribozyme activated RNA-construct(s) of claim 1, wherein the ribozyme activated RNA-construct(s) is linearized and comprises:
a 5′ ribozyme;
a 5′ ligation sequence;
an internal ribosome entry site (IRES) sequence;
an RNA coding sequence for at least one antigenic polypeptide;
a 3′ ligation sequence; and
a 3′ ribozyme sequence, and
a pharmaceutically acceptable carrier.

39. The vaccine composition of claim 38, wherein the linearized ribozyme activated RNA-construct(s) lacks a polymerase binding region.

40. The vaccine composition of claim 38, wherein the 5′ and 3′ ribozyme is selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing.

41. The vaccine composition of claim 38, wherein the 5′ and 3′ ligation sequences are substrates of naturally occurring ligases in situ.

42. The vaccine composition of claim 41, wherein the naturally occurring ligase is RtcB.

43. The vaccine composition of claim 38, wherein the IRES comprises any one of the sequences of SEQ ID NO: 1-1328.

44. The vaccine composition of claim 38, wherein the at least one antigenic polypeptide comprises two or more antigenic polypeptides separated by a self-cleaving peptide.

45. The vaccine composition of claim 44, wherein the self-cleaving peptide comprises a 2A- or 2A-like-peptide.

46. The vaccine composition of claim 45, wherein the 2A- or 2A-like peptide further comprises a GSG linker moiety.

47. The vaccine composition of claim 38, wherein the 5′ and 3′ ribozyme sequences are independently selected from a sequence that is at least 85-100% identical to 5′-GCCATCAGTCGCCGGTCCCAAGCCCGGATAAAATGGGAGGGGGGGGGAAACC GCCT-3′ (SEQ ID NO:1354) or 5′-AACACTGCCAATGCCGGTCCCAAGCCCGGATAAAAGTGGAGGGTACAGTCCAC GC-3′ (SEQ ID NO:1355).

48. The vaccine composition of claim 38, wherein the 5′ and 3′ ligation sequences are independently selected from a sequence that is at least 85-100% identical to 5′-AACCATGCCGACTGATGGCAG-3′ (SEQ ID NO: 1356) or 5′-CTGCCATCAGTCGGCGTGGACTGTAG-3′ (SEQ ID NO: 1357).

49. The vaccine composition of claim 38, wherein the IRES sequence is at least 85-100% identical to 5′-gcggccgcgtcgacgggcccgcggaattccgccccccccccctctccctcccccccccctaacgttactggccgaa gccgcttggaataaggccggtgtgcgtttgtctatatgttattttccaccatattgccgtcttttggcaatgtgagggcccgg aaacctggccctgtcttcttgacgagcattcctaggggtctttcccctctcgccaaaggaatgcaaggtctgttgaatgtc gtgaaggaagcagttcctctggaagcttcttgaagacaaacaacgtctgtagcgaccctttgcaggcagcggaaccc cccacctggcgacaggtgcctctgcggccaaaagccacgtgtataagatacacctgcaaaggcggcacaacccc agtgccacgttgtgagttggatagttgtggaaagagtcaaatggctctcctcaagcgtattcaacaaggggctgaagg atgcccagaaggtaccccattgtatgggatctgatctggggcctcggtgcacatgctttacatgtgtttagtcgaggttaa aaaaacgtctaggccccccgaaccacggggacgtggttttcctttgaaaaacacgatgataatatggccacaacc-3′ (SEQ ID NO:1358).

50. The vaccine composition of claim 38, wherein the antigenic polypeptide comprises a SARS-COV-2 spike protein.

51. The vaccine composition of claim 38, wherein the at least one polypeptide of interest or antigenic polypeptide is contained within a self-amplifying RNA construct.

52. The vaccine composition of claim 51, wherein the self-amplifying RNA construct comprises an alphavirus or a Paramyxovirus.

53. The ribozyme activated RNA-construct(s) of claim 1, wherein the ribozyme activated RNA-construct(s) comprises:
one or more promoter sequences;
one or more RNA coding sequences for at least one polypeptide of interest;
one or more ribozymes, wherein the one or more ribozymes are aptazyme-based riboswitches;
a 3′ UTR sequence comprising the aptazyme-based riboswitches; and
a poly(A) sequence;
wherein the aptazyme-based riboswitches when not bound to target ligands destabilize the ribozyme activated RNA-construct(s) leading to decreased expression of the at least polypeptide of interest, and
wherein the aptazyme-based riboswitches when bound to target ligands stabilize the ribozyme activated RNA-construct(s) leading to increased expression of the at least polypeptide of interest.

54. The ribozyme activated RNA-construct(s) of claim 53, wherein the aptazyme-based riboswitches are hammerhead aptazymes.

55. The ribozyme activated RNA-construct(s) of claim 53, wherein the target ligands are selected from tetracycline, theophylline, and guanine.

56. The ribozyme activated RNA-construct(s) of claim 53, wherein the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

57. The ribozyme activated RNA-construct(s) of claim 56, wherein the biological response modifier or an immunopotentiating cytokine.

58. The ribozyme activated RNA-construct(s) of claim 57, wherein the immunopotentiating cytokine is selected from the group consisting of interleukins 1 through 38, interferon, tumor necrosis factor (TNF), and granulocyte-macrophage-colony stimulating factor (GM-CSF).

59. The ribozyme activated RNA-construct(s) of claim 56, wherein the 2A- or 2A-like peptide further comprises a GSG linker moiety.

60. The ribozyme activated RNA-construct(s) of claim 56, wherein the genome editing enzyme is selected from the group consisting of a zinc finger nuclease, a transcription activator-like effector nuclease (TALEN), an engineered meganuclease and an RNA-guided DNA endonuclease (Cas) polypeptide.

61. The ribozyme activated RNA-construct(s) of claim 53, wherein the one or more promoter sequences are polymerase II (pol-II) promoter sequences.

62. The ribozyme activated RNA-construct(s) of claim 53, wherein the one or more promoter sequences have a sequence(s) for EF1α, hU6, SV40, CMV, a RSV, NEUROD2 and/or TBX20.

63. The ribozyme activated RNA-construct(s) of claim 53, wherein the poly(A) sequence is a bGH poly(A) sequence.

64. A plasmid or capsid comprising the ribozyme activated RNA-construct(s) of claim 53.

65. The plasmid or capsid of claim 64, wherein the plasmid or capsid is an AAV-based plasmid or capsid.

66. The plasmid or capsid of claim 64, wherein the plasmid expresses a Cas9 protein and a gRNA.
